SEQUENCE LISTING (114 sequences)

SEQ ID NO: 1 - DNA, 795 bp, Canis lupus
  source 1..795: mol_type = unassigned DNA
ctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcatgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccctctga

SEQ ID NO: 2 - protein, 264 aa, Canis lupus
LDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 3 - DNA, 438 bp, synthetic construct
  misc_feature 1..438: note = Codon optimized
  source 1..438: mol_type = other DNA
ctggattcccccgacagaccctggagccctctcaccttctcccctgcccagctgaccgtccaggaaggcgagaatgccaccttcacctgcagcctcgccgacatccccgacagcttcgtgctgaactggtacagactgagccccaggaaccagaccgacaagctggccgctttccaggaggacaggatcgaacccggcagggacaggaggtttagggtcatgaggctgcccaacggcagggacttccacatgtccatcgtggccgccagactgaacgactccggcatctacctgtgcggcgctatctacctgccccccaacacccagatcaacgagagccccagggccgaactgagcgtgacagagagaaccctggaacctcccacccagagcccttcccctcctcctagactgagcggacagctgcagggcctggtg

SEQ ID NO: 4 - protein, 438 aa, Canis lupus
CTGGATTCCCCCGACAGACCCTGGAGCCCTCTCACCTTCTCCCCTGCCCAGCTGACCGTCCAGGAAGGCGAGAATGCCACCTTCACCTGCAGCCTCGCCGACATCCCCGACAGCTTCGTGCTGAACTGGTACAGACTGAGCCCCAGGAACCAGACCGACAAGCTGGCCGCTTTCCAGGAGGACAGGATCGAACCCGGCAGGGACAGGAGGTTTAGGGTCATGAGGCTGCCCAACGGCAGGGACTTCCACATGTCCATCGTGGCCGCCAGACTGAACGACTCCGGCATCTACCTGTGCGGCGCTATCTACCTGCCCCCCAACACCCAGATCAACGAGAGCCCCAGGGCCGAACTGAGCGTGACAGAGAGAACCCTGGAACCTCCCACCCAGAGCCCTTCCCCTCCTCCTAGACTGAGCGGACAGCTGCAGGGCCTGGTG

SEQ ID NO: 5 - DNA, 1128 bp, synthetic construct
  misc_feature 1..1128: note = codon optimized canine and human
  source 1..1128: mol_type = other DNA
ctggattcccccgacagaccctggagccctctcaccttctcccctgcccagctgaccgtccaggaaggcgagaatgccaccttcacctgcagcctcgccgacatccccgacagcttcgtgctgaactggtacagactgagccccaggaaccagaccgacaagctggccgctttccaggaggacaggatcgaacccggcagggacaggaggtttagggtcatgaggctgcccaacggcagggacttccacatgtccatcgtggccgccagactgaacgactccggcatctacctgtgcggcgctatctacctgccccccaacacccagatcaacgagagccccagggccgaactgagcgtgacagagagaaccctggaacctcccacccagagcccttcccctcctcctagactgagcggacagctgcagggcctggtgggtaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 375 aa, synthetic construct
  REGION 1..375: note = canine and human
LDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 816 bp, Canis lupus
  source 1..816: mol_type = unassigned DNA
tttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacataa

SEQ ID NO: 8 - protein, 271 aa, Canis lupus
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 9 - DNA, 660 bp, synthetic construct
  misc_feature 1..660: note = codon optimized canine
  source 1..660: mol_type = other DNA
tttaccatcaccgtgtccaaggacctgtacgtggtcgagtacggcggcaatgtgaccatggagtgcaagttccccgtggagaagcagctgaacctgttcgccctcatcgtgtactgggagatggaggacaagaagatcatccagttcgtgaacggcaaggaggacctgaaggtgcagcactccagctactcccagagagcccagctgctgaaggaccagctgttcctgggcaaggccgccctgcagatcaccgacgtgagactgcaggacgccggcgtgtattgctgcctgatcggctacggaggcgccgactacaagaggatcaccctgaaggtgcatgcaccctacaggaacatcagccagaggatcagcgtcgatcccgtgaccagcgagcacgagctgatgtgccaagccgagggctatcccgaggccgaagtgatctggaccagcagcgaccacagggtcctgagcggcaagaccaccatcaccaacagcaacagggaggagaagctgttcaacgtgaccagcaccctcaacatcaacgccaccgccaacgagatcttctactgcaccttccagaggagcggccccgaagagaacaacaccgccgagctggtgatccccgagagactgcctgtgcctgccagcgagaggacccac

SEQ ID NO: 10 - protein, 220 aa, Canis lupus
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTH

SEQ ID NO: 11 - DNA, 1350 bp, synthetic construct
  misc_feature 1..1350: note = codon optimized canine and human
  source 1..1350: mol_type = other DNA
tttaccatcaccgtgtccaaggacctgtacgtggtcgagtacggcggcaatgtgaccatggagtgcaagttccccgtggagaagcagctgaacctgttcgccctcatcgtgtactgggagatggaggacaagaagatcatccagttcgtgaacggcaaggaggacctgaaggtgcagcactccagctactcccagagagcccagctgctgaaggaccagctgttcctgggcaaggccgccctgcagatcaccgacgtgagactgcaggacgccggcgtgtattgctgcctgatcggctacggaggcgccgactacaagaggatcaccctgaaggtgcatgcaccctacaggaacatcagccagaggatcagcgtcgatcccgtgaccagcgagcacgagctgatgtgccaagccgagggctatcccgaggccgaagtgatctggaccagcagcgaccacagggtcctgagcggcaagaccaccatcaccaacagcaacagggaggagaagctgttcaacgtgaccagcaccctcaacatcaacgccaccgccaacgagatcttctactgcaccttccagaggagcggccccgaagagaacaacaccgccgagctggtgatccccgagagactgcctgtgcctgccagcgagaggacccacggtaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 449 aa, synthetic construct
  REGION 1..449: note = canine and human
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 17 aa, Mus musculus
KSSQSLLNSVNQKNYLA

SEQ ID NO: 14 - protein, 16 aa, Mus musculus
RSSQNIVHSNGNTYLE

SEQ ID NO: 15 - protein, 11 aa, Mus musculus
HASQNINVWLS

SEQ ID NO: 16 - protein, 7 aa, Mus musculus
FASTRVS

SEQ ID NO: 17 - protein, 7 aa, Mus musculus
KVSNRFS

SEQ ID NO: 18 - protein, 7 aa, Mus musculus
KASHLHT

SEQ ID NO: 19 - protein, 7 aa, Mus musculus
FASARVS

SEQ ID NO: 20 - protein, 7 aa, Mus musculus
FASTRIS

SEQ ID NO: 21 - protein, 7 aa, Mus musculus
KASNLHT

SEQ ID NO: 22 - protein, 9 aa, Mus musculus
QQYFSTPLT

SEQ ID NO: 23 - protein, 9 aa, Mus musculus
FQGSHVPYT

SEQ ID NO: 24 - protein, 9 aa, Mus musculus
QQGQSWPLT

SEQ ID NO: 25 - protein, 9 aa, Mus musculus
QQYFSTPLT

SEQ ID NO: 26 - protein, 9 aa, Mus musculus
QQGQSYPLT

SEQ ID NO: 27 - protein, 10 aa, Mus musculus
GYTFTTYGMS

SEQ ID NO: 28 - protein, 10 aa, Mus musculus
GYTFTRYNMH

SEQ ID NO: 29 - protein, 10 aa, Mus musculus
GFNIKNTYMH

SEQ ID NO: 30 - protein, 10 aa, Mus musculus
GFSLTSYGVH

SEQ ID NO: 31 - protein, 17 aa, Mus musculus
WINIYSGIPTYADDFKG

SEQ ID NO: 32 - protein, 17 aa, Mus musculus
TIYPGYGDTSYNQKFKG

SEQ ID NO: 33 - protein, 17 aa, Mus musculus
RIAPANVDTKYAPKFQG

SEQ ID NO: 34 - protein, 17 aa, Mus musculus
WINIYSGMPTYADDFKG

SEQ ID NO: 35 - protein, 17 aa, Mus musculus
RIDPANVNTKYAPKFQG

SEQ ID NO: 36 - protein, 6 aa, Mus musculus
FDGPDY

SEQ ID NO: 37 - protein, 13 aa, Mus musculus
EFADDYPIPPFDY

SEQ ID NO: 38 - protein, 11 aa, Mus musculus
IYYDYDGDIDV

SEQ ID NO: 39 - protein, 336 aa, Canis lupus
SASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 40 - protein, 331 aa, synthetic construct
  REGION 1..331: note = modified canine
SASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVFNECRCTDTPPCPAPEMLGGPSVFIFPPKPKATLLIARTPEVTCVVVDLDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 41 - protein, 331 aa, synthetic construct
  REGION 1..331: note = modified canine
SASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKESTCKCISPCPAPEMLGGPSVFIFPPKPKATLLIARTPEVTCVVVDLDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 42 - protein, 336 aa, synthetic construct
  REGION 1..336: note = modified canine
SASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKATLLIARTPEVTCVVVDLDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - DNA, 345 bp, Mus musculus
  source 1..345: mol_type = unassigned DNA
cagatccagttggtacagtctggacctgaactgaagaagcctggagagacagtcaagatctcctgcaaggcttctgggtataccttcacaacctatggaatgagctgggtgaaacaggctccaggaaagggtttaaagtggatgggctggattaatatctactctggaatcccaacatatgctgatgacttcaagggacggtttgccttctctttggaaacctctgccagcactgcctatttgcagatcgacaacctcaaaaatgaggacacggctacatatttctgtgcaagatttgatggtcccgactactggggccaaggcaccactctcaccgtctcccca

SEQ ID NO: 44 - protein, 115 aa, Mus musculus
QIQLVQSGPELKKPGETVKISCKASGYTFTTYGMSWVKQAPGKGLKWMGWINIYSGIPTYADDFKGRFAFSLETSASTAYLQIDNLKNEDTATYFCARFDGPDYWGQGTTLTVSP

SEQ ID NO: 45 - DNA, 339 bp, Mus musculus
  source 1..339: mol_type = unassigned DNA
gacattgtgatgacacagtctccatcctccctggctatgtcagtaggacagaaggtcactatgagctgcaagtccagtcagagccttttaaatagtgtcaatcaaaagaactatttggcctggtaccagcagaaaccaggacagtctcctaaagttctggtatactttgcatccactagggtatctggggtccctgatcgcttcataggcagtggatctgggacagatttcactcttaccatcaccagtgtgcaggctgaagacctgacaacttacctctgtcagcaatattttagcactcctctcacgttcggtgctgggaccaagctggaaataaaa

SEQ ID NO: 46 - protein, 113 aa, Mus musculus
DIVMTQSPSSLAMSVGQKVTMSCKSSQSLLNSVNQKNYLAWYQQKPGQSPKVLVYFASTRVSGVPDRFIGSGSGTDFTLTITSVQAEDLTTYLCQQYFSTPLTFGAGTKLEIK

SEQ ID NO: 47 - DNA, 423 bp, Mus musculus
  source 1..423: mol_type = unassigned DNA
atgggattcagcaggatctttctcttcctcctgtcagtaactacaggtgtccactcccaggcttatctacagcagtctggggctgagctggtgaggcctggggcctcagtgaagatctcctgcaaggcttctggctacacatttaccagatacaatatgcactgggtaaagcagacacctagacagggcctggaatggattggaactatttatcccggatatggtgatacttcttacaatcagaaattcaagggcaaggccacactgactgtagacatatcctccagcacagcctacatgcagctcaccagcctgacatctgaggactctgcggtctatttctgttcaagggagtttgccgatgattaccccattcccccctttgactactggggccaaggcaccactctcacagtctcctca

SEQ ID NO: 48 - protein, 141 aa, Mus musculus
MGFSRIFLFLLSVTTGVHSQAYLQQSGAELVRPGASVKISCKASGYTFTRYNMHWVKQTPRQGLEWIGTIYPGYGDTSYNQKFKGKATLTVDISSSTAYMQLTSLTSEDSAVYFCSREFADDYPIPPFDYWGQGTTLTVSS

SEQ ID NO: 49 - DNA, 393 bp, Mus musculus
  source 1..393: mol_type = unassigned DNA
atgaagttgcctgttaggctgttggtgctgatgttctggattcctgcttccagcagtgatgttttgatgacccaaactccactctccctgcctgtcagtcttggagatcaagcctccatctcttgtagatctagtcagaacattgtacatagtaatggaaacacctacttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggaatttattactgctttcaaggttcacatgttccgtacacgttcggaggggggaccaagctggaaataaaa

SEQ ID NO: 50 - protein, 131 aa, Mus musculus
MKLPVRLLVLMFWIPASSSDVLMTQTPLSLPVSLGDQASISCRSSQNIVHSNGNTYLEWYLQKPGQSPKLLIYKVSNRFSGVPDRFSGSGSGTDFTLKISRVEAEDLGIYYCFQGSHVPYTFGGGTKLEIK

SEQ ID NO: 51 - DNA, 417 bp, Mus musculus
  source 1..417: mol_type = unassigned DNA
atgaaattcagctgggtcatcttcttcctgatggcagtggttacaggggtcaattcagaggttcagctgcagcagtctgtggcagagcttgtgaggccaggggcctcagtcaagttgtcctgcacagcttctggcttcaacattaaaaacacctatatgcactggataaaacagaggcctgaacagggcctggagtggattggaaggattgctcctgcgaatgttgatactaaatatgccccgaagttccagggcaaggccactataactgcagacacatcctccaacacagcctacatgcagctcagcaccctgacatcggaggacactgccatctattactgtgtcctgatctactatgattacgacggggacatcgatgtctggggcacagggaccacggtcaccgtctcctca

SEQ ID NO: 52 - protein, 139 aa, Mus musculus
MKFSWVIFFLMAVVTGVNSEVQLQQSVAELVRPGASVKLSCTASGFNIKNTYMHWIKQRPEQGLEWIGRIAPANVDTKYAPKFQGKATITADTSSNTAYMQLSTLTSEDTAIYYCVLIYYDYDGDIDVWGTGTTVTVSS

SEQ ID NO: 53 - DNA, 381 bp, Mus musculus
  source 1..381: mol_type = unassigned DNA
atgagggtccttgctgagctcctggggctgctgctgttctgctttttaggtgtgagatgtgacatccagatgaaccagtctccatccagtctgtctgcatcccttggagacacaattaccatcacttgccatgccagtcagaacattaatgtttggttaagttggtaccagcagagaccaggaaatattcctaaactattgatctataaggcttctcacttacacacaggcgtcccatcaaggtttagtggcagtggatctggaacaggtttcacattaaccatcagcagcctgcagcctgaagacattgccacttactactgtcaacagggtcaaagttggccgctcacgttcggtgctgggaccaaactggagctgaaa

SEQ ID NO: 54 - protein, 127 aa, Mus musculus
MRVLAELLGLLLFCFLGVRCDIQMNQSPSSLSASLGDTITITCHASQNINVWLSWYQQRPGNIPKLLIYKASHLHTGVPSRFSGSGSGTGFTLTISSLQPEDIATYYCQQGQSWPLTFGAGTKLELK

SEQ ID NO: 55 - DNA, 402 bp, Mus musculus
  source 1..402: mol_type = unassigned DNA
atgggttggctgtggaacttgctattcctgatggcagctgcccaaagtgcccaaacacagatccagttggtacagtctggacctgaactgaagaagcctggagagacagtcaagatctcctgcaaggcttctgggtataccttcacaacctatggaatgagctgggtgaaacaggctccaggaaagggtttaaagtggatgggctggattaatatctactctggaatcccaacatatgctgatgacttcaagggacgatttgccttctctttggaaacctctgccagcactgcctatttgcagatcgacaacctcaaaaatgaggacacggctacatatttctgtgcaagatttgatggtcccgactactggggccaaggcaccactctcacagtctcctca

SEQ ID NO: 56 - protein, 134 aa, Mus musculus
MGWLWNLLFLMAAAQSAQTQIQLVQSGPELKKPGETVKISCKASGYTFTTYGMSWVKQAPGKGLKWMGWINIYSGIPTYADDFKGRFAFSLETSASTAYLQIDNLKNEDTATYFCARFDGPDYWGQGTTLTVSS

SEQ ID NO: 57 - DNA, 399 bp, Mus musculus
  source 1..399: mol_type = unassigned DNA
atggaatcacagacccaggtcctcatgtttcttctgctctgggtatctggtgcctgtgcagacattgtgatgacacagtctccatcctccctggctgtgtcagtaggacggaaggtcactatgagctgcaagtccagtcagagccttttaaatagtgtcaatcaaaagaactatttggcctggtaccagcagaaaccaggacagtctcctaaagttctggtatactttgcatccgctagggtatctggggtccctgatcgcttcataggcagtggatctgggacagatttcactcttgccatcagcagtgtgcaggctgaagacctgacaacttacttctgtcagcaatattttagcactcctctcacgttcggtgctgggaccaagctggaactgaaa

SEQ ID NO: 58 - protein, 133 aa, Mus musculus
MESQTQVLMFLLLWVSGACADIVMTQSPSSLAVSVGRKVTMSCKSSQSLLNSVNQKNYLAWYQQKPGQSPKVLVYFASARVSGVPDRFIGSGSGTDFTLAISSVQAEDLTTYFCQQYFSTPLTFGAGTKLELK

SEQ ID NO: 59 - DNA, 402 bp, Mus musculus
  source 1..402: mol_type = unassigned DNA
atgggttggctgtggaacttgctattcctgatggcagctgcccaaagtgcccaagcacagatccagttggtacagtctggacctgaactgaagaagcctggagagacagtcaagatctcctgcaaggcttctgggtataccttcacaacctatggaatgagctgggtgaaacaggcgccaggaaagggtttaaagtggatgggctggataaatatctactctggaatgccaacatatgctgatgacttcaagggacggtttgccttctctttggaaacctctgtcagcactgcctatttgcagatcaacaacctcaaaaatgaggacacggctacatatttctgtgcaagatttgatggtcccgactactggggccaaggcaccactctcacagtctcctca

SEQ ID NO: 60 - protein, 134 aa, Mus musculus
MGWLWNLLFLMAAAQSAQAQIQLVQSGPELKKPGETVKISCKASGYTFTTYGMSWVKQAPGKGLKWMGWINIYSGMPTYADDFKGRFAFSLETSVSTAYLQINNLKNEDTATYFCARFDGPDYWGQGTTLTVSS

SEQ ID NO: 61 - DNA, 399 bp, Mus musculus
  source 1..399: mol_type = unassigned DNA
atggaatcacagacccaggtcctcatgtttcttctgctctgggtatctggtgcctgtgcagacattgtgatgacacagtctccatcctccctggctatgtcagtaggacagaaggtcactatgagctgcaagtccagtcagagccttttaaatagtgtcaatcaaaagaactatttggcctggtaccagcagaaaccaggacagtctcctaaagttctggtatactttgcatccactaggatatctggggtccctgatcgcttcataggcagtggatctgggacagatttcactcttaccatcagcagtgtgcaggctgaagacctggcagattacttctgtcagcaatattttagcactcctctcacgttcggtgctgggaccaagctggagctgaaa

SEQ ID NO: 62 - protein, 133 aa, Mus musculus
MESQTQVLMFLLLWVSGACADIVMTQSPSSLAMSVGQKVTMSCKSSQSLLNSVNQKNYLAWYQQKPGQSPKVLVYFASTRISGVPDRFIGSGSGTDFTLTISSVQAEDLADYFCQQYFSTPLTFGAGTKLELK

SEQ ID NO: 63 - DNA, 417 bp, Mus musculus
  source 1..417: mol_type = unassigned DNA
atgaaattcagctgggtcatcttcttcctgatggcagtggttacaggggtcaattcagaggttcagctgcagcagtctgtggcagagcttgtgaggccaggggcctcagtcaagttgtcctgcacagtttctggcttcaacattaaaaacacctatatgcactgggtgaagcagaggcctgaacagggcctggagtggattggaagaattgatcctgcgaatgttaatactaaatatgccccgaagttccagggcaaggccactataactacagacacatcctccaacacagcctacatgcagctcagcagcctgacatcggaggacactgccatctattactgtgtcctgattttctatgattacgacggggacatcgatgtctggggcacagggaccaaggtcaccgtctcctca

SEQ ID NO: 64 - protein, 139 aa, Mus musculus
MKFSWVIFFLMAVVTGVNSEVQLQQSVAELVRPGASVKLSCTVSGFNIKNTYMHWVKQRPEQGLEWIGRIDPANVNTKYAPKFQGKATITTDTSSNTAYMQLSSLTSEDTAIYYCVLIFYDYDGDIDVWGTGTKVTVSS

SEQ ID NO: 65 - DNA, 381 bp, Mus musculus
  source 1..381: mol_type = unassigned DNA
atgagggtccttgctgagctcctggggctgctgctgttctgctttttaggtgtgagatgtgacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttacacacaggcgtcccatcaaggtttagtggcagtggatctggaacagatttcacattaaccatcagcagcctgcagcctgaagacattgccacttactactgtcaacagggtcaaagttatccgctcacgttcggtgctgggaccaagctggagctgaaa

SEQ ID NO: 66 - protein, 127 aa, Mus musculus
MRVLAELLGLLLFCFLGVRCDIQMNQSPSSLSASLGDTITITCHASQNINVWLSWYQQKPGNIPKLLIYKASNLHTGVPSRFSGSGSGTDFTLTISSLQPEDIATYYCQQGQSYPLTFGAGTKLELK

SEQ ID NO: 67 - DNA, 345 bp, Mus musculus
  source 1..345: mol_type = unassigned DNA
caggtgcagctgaagcagtcaggacctggcctagtgcagccctcacagagcctgtccataacctgcacagtctctggtttctcattaactagctatggtgtacactgggttcgccagtctccaggaaagggtttaaagtggatgggctggattaatatctactctggaatcccaacatatgctgatgacttcaagggacggtttgccttctctttggaaacctctgccagcactgcctatttgcagatcgacaacctcaaaaatgaggacacggctacatatttctgtgcaagatttgatggtcccgactactggggccaaggcatcactctcactgtctccgca

SEQ ID NO: 68 - protein, 115 aa, Mus musculus
QVQLKQSGPGLVQPSQSLSITCTVSGFSLTSYGVHWVRQSPGKGLKWMGWINIYSGIPTYADDFKGRFAFSLETSASTAYLQIDNLKNEDTATYFCARFDGPDYWGQGITLTVSA

SEQ ID NO: 69 - DNA, 339 bp, Mus musculus
  source 1..339: mol_type = unassigned DNA
gacattgtgatgacacagtctccatcctccctggctatgtcagtaggacagaaggtcactatgagctgcaagtccagtcagagccttttaaatagtgtcaatcaaaagaactatttggcctggtaccagcagaaaccaggacagtctcctaaagttctggtatactttgcatccactagggtatctggggtccctgatcgcttcataggcagtggatctgggacagatttcactcttaccatcaccagtgtgcaggctgaagacctgacaacttacttctgtcagcaatattttagcactcctctcacgttcggtgctgggaccaagctggaactgaaa

SEQ ID NO: 70 - protein, 113 aa, Mus musculus
DIVMTQSPSSLAMSVGQKVTMSCKSSQSLLNSVNQKNYLAWYQQKPGQSPKVLVYFASTRVSGVPDRFIGSGSGTDFTLTITSVQAEDLTTYFCQQYFSTPLTFGAGTKLELK

SEQ ID NO: 71 - protein, 15 aa, Canis lupus
LDSPDRPWSPLTFSP

SEQ ID NO: 72 - protein, 15 aa, Canis lupus
RPWSPLTFSPAQLTV

SEQ ID NO: 73 - protein, 15 aa, Canis lupus
LTFSPAQLTVQEGEN

SEQ ID NO: 74 - protein, 15 aa, Canis lupus
AQLTVQEGENATFTC

SEQ ID NO: 75 - protein, 15 aa, Canis lupus
QEGENATFTCSLADI

SEQ ID NO: 76 - protein, 15 aa, Canis lupus
ATFTCSLADIPDSFV

SEQ ID NO: 77 - protein, 15 aa, Canis lupus
SLADIPDSFVLNWYR

SEQ ID NO: 78 - protein, 15 aa, Canis lupus
PDSFVLNWYRLSPRN

SEQ ID NO: 79 - protein, 15 aa, Canis lupus
LNWYRLSPRNQTDKL

SEQ ID NO: 80 - protein, 15 aa, Canis lupus
LSPRNQTDKLAAFQE

SEQ ID NO: 81 - protein, 15 aa, Canis lupus
QTDKLAAFQEDRIEP

SEQ ID NO: 82 - protein, 15 aa, Canis lupus
AAFQEDRIEPGRDRR

SEQ ID NO: 83 - protein, 15 aa, Canis lupus
  SITE 14: note = MISC_FEATURE - Xaa can be Met or Thr
DRIEPGRDRRFRVXR

SEQ ID NO: 84 - protein, 15 aa, Canis lupus
  SITE 9: note = MISC_FEATURE - Xaa can be Met or Thr
GRDRRFRVXRLPNGR

SEQ ID NO: 85 - protein, 15 aa, Canis lupus
  SITE 4: note = MISC_FEATURE - Xaa can be Met or Thr
FRVXRLPNGRDFHMS

SEQ ID NO: 86 - protein, 15 aa, Canis lupus
LPNGRDFHMSIVAAR

SEQ ID NO: 87 - protein, 15 aa, Canis lupus
DFHMSIVAARLNDSG

SEQ ID NO: 88 - protein, 15 aa, Canis lupus
IVAARLNDSGIYLCG

SEQ ID NO: 89 - protein, 15 aa, Canis lupus
LNDSGIYLCGAIYLP

SEQ ID NO: 90 - protein, 15 aa, Canis lupus
IYLCGAIYLPPNTQI

SEQ ID NO: 91 - protein, 15 aa, Canis lupus
AIYLPPNTQINESPR

SEQ ID NO: 92 - protein, 15 aa, Canis lupus
PNTQINESPRAELSV

SEQ ID NO: 93 - protein, 15 aa, Canis lupus
NESPRAELSVTERTL

SEQ ID NO: 94 - protein, 15 aa, Canis lupus
AELSVTERTLEPPTQ

SEQ ID NO: 95 - protein, 15 aa, Canis lupus
TERTLEPPTQSPSPP

SEQ ID NO: 96 - protein, 15 aa, Canis lupus
EPPTQSPSPPPRLSG

SEQ ID NO: 97 - protein, 15 aa, Canis lupus
SPSPPPRLSGQLQGL

SEQ ID NO: 98 - protein, 15 aa, Canis lupus
PSPPPRLSGQLQGLV

SEQ ID NO: 99 - protein, 25 aa, Canis lupus
  SITE 5: note = MISC_FEATURE - Xaa can be Met or Thr
RFRVXRLPNGRDFHMSIVAARLNDS

SEQ ID NO: 100 - protein, 26 aa, Canis lupus
  SITE 20: note = misc_feature - Xaa can be any naturally occurring amino acid
LAAFQEDRIEPGRDRRFRVXRLPNGR

SEQ ID NO: 101 - protein, 31 aa, Canis lupus
  SITE 15: note = MISC_FEATURE - Xaa can be Met or Thr
EDRIEPGRDRRFRVXRLPNGRDFHMSIVAAR

SEQ ID NO: 102 - protein, 31 aa, Canis lupus
  SITE 25: note = MISC_FEATURE - Xaa can be Met or Thr
NQTDKLAAFQEDRIEPGRDRRFRVXRLPNGR

SEQ ID NO: 103 - protein, 45 aa, Canis lupus
  SITE 25: note = MISC_FEATURE - Xaa can be Met or Thr
NQTDKLAAFQEDRIEPGRDRRFRVXRLPNGRDFHMSIVAARLNDS

SEQ ID NO: 104 - protein, 20 aa, Canis lupus
  SITE 14: note = misc_feature - Xaa can be Met or Thr
DRIEPGRDRRFRVXRLPNGR

SEQ ID NO: 105 - DNA, 867 bp, Canis lupus
  source 1..867: mol_type = unassigned DNA
atggggagccggcgggggccctggccgctcgtctgggccgtgctgcagctgggctggtggccaggatggctcctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcatgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccctctga

SEQ ID NO: 106 - protein, 288 aa, Canis lupus
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 107 - DNA, 870 bp, Canis lupus
  source 1..870: mol_type = unassigned DNA
atgagaatgtttagtgtctttacattcatggcctactgccatttgctaaaagcatttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacataa

SEQ ID NO: 108 - protein, 289 aa, Canis lupus
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 109 - DNA, 423 bp, Mus musculus
  source 1..423: mol_type = unassigned DNA
atgggattcagcaggatctttctcttcctcctgtcagtaactacaggtgtccactcccaggcttatctacagcagtctggggctgagctggtgaggcctggggcctcagtgaagatgtcctgcaaggcttttggctacacatttaccagttacaatatgcactgggtgaagcagacacctagacagggcctggaatggattggaaccatttatccaggagatggtgacgcttcctacaatcagaaattccaggacaaggccacactgactgttgacaaatcctccagcacagcctacatgcagctcagcagcctgacatctgaagactctgcggtctatttctgttcaagggagtttgccgatgcttaccccattcccccctttgactactggggccaaggcaccactctcacagtctcctca

SEQ ID NO: 110 - protein, 141 aa, Mus musculus
MGFSRIFLFLLSVTTGVHSQAYLQQSGAELVRPGASVKMSCKAFGYTFTSYNMHWVKQTPRQGLEWIGTIYPGDGDASYNQKFQDKATLTVDKSSSTAYMQLSSLTSEDSAVYFCSREFADAYPIPPFDYWGQGTTLTVSS

SEQ ID NO: 111 - DNA, 393 bp, Mus musculus
  source 1..393: mol_type = unassigned DNA
atgaagttgcctgttaggctgttggtgctgattttctggattcctgcttccagtagtgatgttttgatgacccaaactccactctccctggttgtcagtcttggagatcaggcctccatctcttgcagatctagtcagagcattgtatatagtaatggaaacacctatttagaatggtacctgcaaaaaccaggccagtctccaaagctcctgatttacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 112 - protein, 131 aa, Mus musculus
MKLPVRLLVLIFWIPASSSDVLMTQTPLSLVVSLGDQASISCRSSQSIVYSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 113 - protein, 401 aa, synthetic construct
  REGION 1..401: note = canine and human
MNFLLSWVHWSLALLLYLHHAKWSQALDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 11 aa, Mus musculus
IFYDYDGDIDV

(19) United States
(12) Patent Application Publication
(10) Pub. No.: US 2024/0084004 A1
(43) Pub. Date: Mar. 14, 2024

(54) ANTIBODIES AGAINST CANINE PD-1

(71) Applicant: Intervet Inc., Madison, NJ (US)

(72) Inventors: Mohamad Morsey, Omaha, NE (US); Yuanzheng Zhang, Edison, NJ (US); Denise Bartels-Morozov, Fremont, NE (US); Jason Erskine, Omaka, NE (US); Ian Tarpey, St. Ives (GB)

(73) Assignee: Intervet Inc., Madison, NJ (US)

(21) Appl. No.: 18/313,759

(22) Filed: May 8, 2023

Related U.S. Application Data
(63) Continuation of application No. 16/893,529, filed on Jun. 5, 2020, now Pat. No. 11,680,097.
Application No. 16/893,529 is a division of application No. 15/894,493, filed on Feb. 12, 2018, now Pat. No. 10,711,061.
Application No. 15/894,493 is a continuation of application No. 15/104,844, filed on Jun. 15, 2016, now Pat. No. 9,944,704.
(60) Provisional application No. 61/918,946, filed on Dec. 20, 2013.
(60) Provisional application No. 61/918,847, filed on Dec. 20, 2013.
(60) Provisional application No. 62/030,812, filed on Jul. 30, 2014.

(51) Int. Cl. C07K 16/28; A61K 39/00; A61K 39/395; A61P 37/04

(52) U.S. Cl. CPC C07K 16/2803; A61K 39/00; A61K 39/3955; A61P 37/04; C07K 16/2818; C07K 16/2896; A61K 2039/55516; C07K 2317/24; C07K 2317/34; C07K 2317/52; C07K 2317/565; C07K 2317/76; C07K 2317/92

(57) ABSTRACT

The present invention discloses antibodies to canine PD-I that have specific sequences and a high binding affinity for canine PD-I. The invention also discloses the use of the antibodies of the present invention in the treatment of cancer in dogs.

### CROSS-REFERENCE TO RELATED APPLICATIONS

This application claims benefit of U.S. Provisional Application Ser. No. 61/918,946, filed on Dec. 20, 2013, U.S. Provisional Application Ser. No. 61/918,847, filed on Dec. 20, 2013, and U.S. Provisional Application Ser. No. 62/030,812, filed on Jul. 30, 2014, the contents of all of which are hereby incorporated by reference in their entireties.

### FIELD OF THE INVENTION

The present invention relates to murine antibodies to canine PD-1 that have specific sequences and a high binding affinity for canine PD-1. The invention also relates to use of the antibodies of the present invention in the treatment of cancer in dogs.

### BACKGROUND OF THE INVENTION

An immunoinhibitory receptor that is primarily expressed on activated T and B cells, Programmed Cell Death Receptor 1, also referred to as Programmed Death Receptor 1 (PD-1), is a member of the immunoglobulin superfamily related to CD28 and CTLA-4. PD-1 and like family members are type I transmembrane glycoproteins containing an extracellular Ig Variable-type (V-type) domain that binds its ligands and a cytoplasmic tail that binds signaling molecules. The cytoplasmic tail of PD-1 contains two tyrosine-based signaling motifs, an ITIM (immunoreceptor tyrosine-based inhibition motif) and an ITSM (immunoreceptor tyrosine-based switch motif).

PD-1 attenuates T-cell responses when bound to Programmed Cell Death Ligand 1, also referred to as Programmed Death Ligand 1 (PD-L1), and/or Programmed Cell Death Ligand 2, also referred to as Programmed Death Ligand 2 (PD-L2). The binding of either of these ligands to PD-1 negatively regulates antigen receptor signaling. Blocking the binding of PD-L1 to PD-1 enhances tumor-specific CD8⁺ T-cell immunity, while aiding the clearance of tumor cells by the immune system. The three-dimensional structure of murine PD-1, as well as the co-crystal structure of mouse PD-1 with human PD-L1 have been reported [Zhang et al., Immunity 20: 337-347 (2004); Lin et al., Proc. Natl. Acad. Sci. USA 105: 3011-3016 (2008)]. PD-L1 and PD-L2 are type I transmembrane ligands that contain both IgV- and IgC-like domains in the extracellular region along with short cytoplasmic regions with no known signaling motifs. Both PD-L1 and PD-L2 are either constitutively expressed or can be induced in a variety of cell types, including non-hematopoietic tissues as well as various tumor types. PD-L1 is not only expressed on B, T, myeloid and dendritic cells (DCs), but also on peripheral cells, such as microvascular endothelial cells and non-lymphoid organs e.g., heart or lung. In contrast, PD-L2 is only found on macrophages and DCs. The expression pattern of PD-1 ligands suggests that PD-1 plays a role in maintaining peripheral tolerance and may further serve to regulate self-reactive T- and B-cell responses in the periphery.

In any case, it is now abundantly clear that PD-1 plays a critical role in at least certain human cancers, presumably by mediating immune evasion. Accordingly, PD-L1 has been shown to be expressed on a number of mouse and human tumors and is inducible by IFN gamma in the majority of PD-L1 negative tumor cell lines [Iwai et al., Proc. Natl. Acad. Sci. U.S.A. 99: 12293-12297 (2002); Strome et al., Cancer Res., 63: 6501-6505 (2003)]. Furthermore, the expression of PD-1 on tumor infiltrating lymphocytes and/or PD-L1 on tumor cells has been identified in a number of primary human tumor biopsies. Such tumor tissues include cancers of the lung, liver, ovary, cervix, skin, colon, glioma, bladder, breast, kidney, esophagus, stomach, oral squamous cell, urothelial cell, and pancreas, as well as tumors of the head and neck [Brown et al., J. Immunol. 170: 1257-1266 (2003); Dong et al., Nat. Med. 8: 793-800 (2002); Wintterle et al., Cancer Res. 63: 7462-7467 (2003); Strome et al., Cancer Res., 63: 6501-6505 (2003); Thompson et al., Cancer Res. 66: 3381-5 (2006); Thompson et al., Clin. Cancer Res. 13: 1757-1761 (2007); Nomi et al., Clin. Cancer Res. 13: 2151-2157. (2007)]. More strikingly, PD-ligand expression on tumor cells has been correlated to poor prognosis of human cancer patients across multiple tumor types [reviewed in Honjo, Int. Immunol. 19: 813-824 (2007)].

Moreover, Nomi et al. [Clin Cancer Res. 13: 2151-2157(2007)] demonstrated the therapeutic efficacy of blocking the binding of PD-L1 to PD-1 in a murine model of eaggressive pancreatic cancer through administering either PD-1 or PD-L1 directed antibody. These antibodies effectively promoted tumor reactive CD8+ T cell infiltration into the tumor resulting in the up-regulation of anti-tumor effectors including IFN gamma, granzyme B, and perforing. Similarly, the use of antibodies to block the binding of PD-L1 and PD-1 significantly inhibited tumor growth in a model of mouse squamous cell carcinoma [Tsushima et al., Oral Oncol. 42: 268-274 (2006)].

In other studies, transfection of a murine mastocytoma line with PD-L1 led to decreased lysis of the tumor cells when co-cultured with a tumor-specific CTL clone. Lysis was restored when anti-PD-L1 monoclonal antibody was added [Iwai et al., Proc. Natl. Acad. Sci U.S.A. 99: 12293-12297 (2002)]. In vivo, blocking the PD1/PD-L1 interaction was shown to increase the efficacy of adoptive T cell transfer therapy in a mouse tumor model [Strome et al., Cancer Res. 63: 6501-6505 (2003)]. Further evidence for the role of PD-1 in cancer treatment comes from experiments performed with PD-1 knockout mice in which PD-L1 expressing myeloma cells grew only in wild-type animals (resulting in tumor growth and associated animal death), but not in PD-1 deficient mice [Iwai et al., Proc. Natl. Acad. Sci U.S.A. 99: 12293-12297 (2002)]. More recently, antibodies against PD-1 (including humanized murine monoclonal antibodies against human PD-1) have shown at least initial success in cancer therapy in humans [see e.g., U.S. Pat. No. 8,354,509 B2, U.S. Pat. No. 8,008,449 B2, and U.S. Pat. No. 7,595,048 B2].

Anti-PD-1 antibodies may also be useful in chronic viral infection. Memory CD8⁺ T cells generated after an acute viral infection are highly functional and constitute an important component of protective immunity. In contrast, chronic infections are often characterized by varying degrees of functional impairment (exhaustion) of virus-specific T-cell responses, and this defect is a principal reason for the inability of the host to eliminate the persisting pathogen. Although functional effector T cells are initially generated during the early stages of infection, they gradually lose function during the course of a chronic infection. Barber et al. [Nature 439: 682-687 (2006)] showed that mice infected with a laboratory strain of LCMV developed chronic infection resulted in high levels of virus in the blood and other tissues. These mice initially developed a robust T cell response, but eventually succumbed to the infection upon T cell exhaustion. Barber et al. found that the decline in number and function of the effector T cells in chronically infected mice could be reversed by injecting an antibody that blocked the interaction between PD-1 and PD-L1.

The citation of any reference herein should not be construed as an admission that such reference is available as “prior art” to the instant application.

### SUMMARY OF THE INVENTION

The present invention relates to anti-canine PD-1 antibodies that have a high binding affinity to canine PD-1, as well as have the ability to block the binding of canine PD-1 to canine PD-L1. In particular embodiments such anti-canine PD-1 antibodies are murine anti-canine PD-1 antibodies. In particular embodiments the anti-canine PD-1 antibodies have a high binding affinity to canine PD-1, as well as have the ability to also block the binding of canine PD-1 to canine PD-L2.

Moreover, the present invention relates to the complementary determining regions (CDRs) comprised by these antibodies and the combination of these CDRs (e.g., obtained from murine anti-canine PD-1 antibodies) into canine frames to form caninized anti-canine PD-1 antibodies. The present invention also relates to use of such antibodies in the treatment of disease such as cancer and/or those due to infections.

Accordingly, the present invention provides unique sets of CDRs from seven exemplified murine anti-canine PD-1 antibodies. Although each of the seven exemplified murine anti-canine PD-1 antibodies have a unique set of CDRs, i.e., three light chain CDRs: CDR light 1 (CDRL1), CDR light 2 (CDRL2), and CDR light 3 (CDRL3) and three heavy chain CDRs CDR heavy 1 (CDRH1), CDR heavy 2 (CDRH2) and CDR heavy 3 (CDRH3), as detailed below, there is substantial sequence homology within each group of CDRs, e.g., the set of CDRL1s. Therefore, the present invention not only provides the amino acid sequences of the six CDRs from seven exemplified murine anti-canine PD-1 antibodies, but further provides conservatively modified variants of those CDRs, as well as variants that comprise (e.g., share) the same canonical structure and/or bind to one or more (e.g., 1 to 4, or even all) amino acid residues of canine PD-1 that are comprised by an epitope of canine PD-1.

Therefore, the present invention provides an antibody or antigen binding fragment thereof that binds canine Programmed Death Receptor 1 (canine PD-1) with specificity comprising a light chain complementary determining region 1 (VL CDR1) that comprises the amino acid sequence of SEQ ID NO: 13, SEQ ID NO: 14, or SEQ ID NO: 15, and/or a light chain complementary determining region 2 (VL CDR2) comprising the amino acid sequence of SEQ ID NO: 16, SEQ ID NO: 17, SEQ ID NO: 18, SEQ ID NO: 19, SEQ ID NO: 20, or SEQ ID NO: 21, and/or a light chain complementary determining region 3 (VL CDR3) comprising the amino acid sequence of SEQ ID NO: 22, SEQ ID NO: 23, SEQ ID NO: 24, SEQ ID NO: 25, or SEQ ID NO: 26, and/or a heavy chain complementary determining region 1 (VH CDR1) in which the CDRH1 comprises the amino acid sequence of SEQ ID NO: 27, SEQ ID NO: 28, SEQ ID NO: 29, or SEQ ID NO: 30, and/or a heavy chain complementary determining region 2 (VH CDR2) comprising the amino acid sequence of SEQ ID NO: 31, SEQ ID NO: 32, SEQ ID NO: 33, SEQ ID NO: 34, or SEQ ID NO: 35, and/or a heavy chain complementary determining region 3 (VH CDR3) comprising the amino acid sequence of SEQ ID NO: 36, SEQ ID NO: 37, SEQ ID NO: 38, or SEQ ID NO: 114. In particular embodiments the antibody is a mammalian antibody. In more particular embodiments the antibody is a caninized antibody.

Accordingly, a caninized antibody of the present invention or antigen binding fragment thereof comprises one or more of the heavy chain complementary determining region 1 (VH CDR1) with an amino acid sequence of SEQ ID NO: 27, SEQ ID NO: 28, SEQ ID NO: 29, or SEQ ID NO: 30. In another embodiment, the heavy chain complementary determining region 2 (VH CDR2) comprises an amino acid sequence of SEQ ID NO: 31, SEQ ID NO: 32, SEQ ID NO: 33, SEQ ID NO: 34, or SEQ ID NO: 35. In still another embodiment the heavy chain complementary determining region 3 (VH CDR3) comprises an amino acid sequence of SEQ ID NO: 36, SEQ ID NO: 37, SEQ ID NO: 38, or SEQ ID NO: 114. In a particular embodiment of this type, the caninized antibody or antigen binding fragment comprises both a VH CDR1 comprising an amino acid sequence of SEQ ID NO: 27, SEQ ID NO: 28, SEQ ID NO: 29, or SEQ ID NO: 30 and a VH CDR2 comprising an amino acid sequence of SEQ ID NO: 31, SEQ ID NO: 32, SEQ ID NO: 33, SEQ ID NO: 34, or SEQ ID NO: 35. In another such embodiment, the caninized antibody or antigen binding fragment comprises both a VH CDR1 comprising an amino acid sequence of SEQ ID NO: 27, SEQ ID NO: 28, SEQ ID NO: 29, or SEQ ID NO: 30 and a VH CDR3 comprising an amino acid sequence of SEQ ID NO: 36, SEQ ID NO: 37, SEQ ID NO: 38, or SEQ ID NO: 114. In yet another such embodiment, the caninized antibody or antigen binding fragment comprises both a VH CDR2 comprising an amino acid sequence of SEQ ID NO: 31, SEQ ID NO: 32, SEQ ID NO: 33, SEQ ID NO: 34, or SEQ ID NO: 35 and a VH CDR3 comprising an amino acid sequence of SEQ ID NO: 36, SEQ ID NO: 37, SEQ ID NO: 38, or SEQ ID NO: 114. In still another such embodiment, the caninized antibody or antigen binding fragment comprises a VH CDR1 comprising an amino acid sequence of SEQ ID NO: 27, SEQ ID NO: 28, SEQ ID NO: 29, or SEQ ID NO: 30, a VH CDR2 comprising an amino acid sequence of SEQ ID NO: 31, SEQ ID NO: 32, SEQ ID NO: 33, SEQ ID NO: 34, or SEQ ID NO: 35, and a VH CDR3 comprising an amino acid sequence of SEQ ID NO: 36, SEQ ID NO: 37, SEQ ID NO: 38, or SEQ ID NO: 114.

In particular embodiments, the caninized antibody or antigen binding fragment also comprises a light chain complementary determining region 1 (VL CDR1) comprising an amino acid sequence of SEQ ID NO: 13, SEQ ID NO: 14, or SEQ ID NO: 15. In related embodiments the light chain complementary determining region 2 (VL CDR2) comprises an amino acid sequence of SEQ ID NO: 16, SEQ ID NO: 17, SEQ ID NO: 18, SEQ ID NO: 19, SEQ ID NO: 20, or SEQ ID NO: 21. In still another embodiment the light chain complementary determining region 3 (VL CDR3) comprises an amino acid sequence of SEQ ID NO: 22, SEQ ID NO: 23, SEQ ID NO: 24, SEQ ID NO: 25, or SEQ ID NO: 26. In a particular embodiment of this type, the caninized antibody or antigen binding fragment comprises both a VL CDR1 comprising an amino acid sequence of SEQ ID NO: 13, SEQ ID NO: 14, or SEQ ID NO: 15 and a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21.

In other such embodiments, the caninized antibody or antigen binding fragment comprises both a VL CDR1 comprising an amino acid sequence of SEQ ID NO: 13, SEQ ID NO: 14, or SEQ ID NO: 15 and a VL CDR3 comprising an amino acid sequence of SEQ ID NO: 22, SEQ ID NO: 23, SEQ ID NO: 24, SEQ ID NO: 25, or SEQ ID NO: 26. In yet another such embodiments, the caninized antibody or antigen binding fragment comprises both a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21 and a VL CDR3 comprising an amino acid sequence of SEQ ID NO: 22, SEQ ID NO: 23, SEQ ID NO: 24, SEQ ID NO: 25, or SEQ ID NO: 26. In still other such embodiments, the caninized antibody or antigen binding fragment comprises a VL CDR1 comprising an amino acid sequence of SEQ ID NO: 13, SEQ ID NO: 14, or SEQ ID NO: 15, a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21, and a VL CDR3 comprising an amino acid sequence of SEQ ID NO: 22, SEQ ID NO: 23, SEQ ID NO: 24, SEQ ID NO: 25, or SEQ ID NO: 26.

In particular embodiments the caninized anti-canine PD-1 antibody further comprises complementary determining regions (CDRs) in which the CDRs have canonical structures of: H1-1, H2-1, and H3-6, respectively for CDR1, CDR2, and CDR3 of the heavy chain, i.e., CDR1 of the heavy chain has the canonical structure class 1, CDR2 of the heavy chain has the canonical structure class 1, and CDR3 of the heavy chain has the canonical structure class 6. In even more particular embodiments, the CDRs for the corresponding light chains have canonical structures of: L1-3, L2-1, and L3-1, respectively for CDR1, CDR2, and CDR3 of the light chain. In other embodiments the caninized anti-canine PD-1 antibody further comprises complementary determining regions (CDRs) in which the CDRs have canonical structures of: H1-1, H2-1, and H3-11, respectively for CDR1, CDR2, and CDR3 of the heavy chain. In even more particular embodiments of this type, the CDRs for the corresponding light chains have canonical structures of: L1-2A, L2-1, and L3-1, respectively for CDR1, CDR2, and CDR3 of the light chain. In still other embodiments the caninized anti-canine PD-1 antibody further comprises complementary determining regions (CDRs) in which the CDRs have canonical structures of: H1-1, H2-2A, and H3-11, respectively for CDR1, CDR2, and CDR3 of the heavy chain. In even more particular embodiments of this type, the CDRs for the corresponding light chains have canonical structures of: L1-2A, L2-1, and L3-1, respectively for CDR1, CDR2, and CDR3 of the light chain. In yet other embodiments the caninized anti-canine PD-1 antibody further comprises complementary determining regions (CDRs) in which the CDRs have canonical structures of: H1-1, H2-2A, and H3-13, respectively for CDR1, CDR2, and CDR3 of the heavy chain. In even more particular embodiments of this type, the CDRs for the corresponding light chains have canonical structures of: L1-4, L2-1, and L3-1, respectively for CDR1, CDR2, and CDR3 of the light chain.

Furthermore, the present invention provides antibodies to canine PD-1, e.g., monoclonal antibodies, that comprise variants of the CDRs of the present invention that have the corresponding canonical structures provided herein and that bind to the amino acid sequence of SEQ ID NO: 103. In particular embodiments of this type, the dissociation constant (Kd) for antibody-canine PD-1 binding is 1×10⁻⁵ to 1×10⁻¹² M. In more particular embodiments the antibodies to canine PD-1, comprise variants of the CDRs of the present invention that have the corresponding canonical structures provided herein and bind to the amino acid sequence of SEQ ID NO: 104.

The present invention also provides an isolated caninized antibody or antigen binding fragment thereof that specifically binds Programmed Death Receptor 1 (PD-1) comprising a canine IgG heavy chain and a canine kappa or lambda light chain. In particular embodiments of this type, the canine kappa or lambda light chain that comprises three light chain complementary determining regions (CDRs): CDR light 1 (CDRL1), CDR light 2 (CDRL2), and CDR light 3 (CDRL3); and the canine IgG heavy chain comprises three heavy chain CDRs: CDR heavy 1 (CDRH1), CDR heavy 2 (CDRH2) and CDR heavy 3 (CDRH3) is obtained from the murine anti-canine PD-1 antibodies. Particular embodiments of the caninized antibodies and antigen binding fragments thereof of the present invention bind canine PD-1 and/or block the binding of canine PD-1 to canine Programmed Death Ligand 1 (PD-L1).

In specific embodiments, the present invention provides an isolated mammalian antibody or antigen binding fragment thereof that binds canine Programmed Death Receptor 1 (canine PD-1) with specificity comprising three light chain complementary determining regions (CDRs): CDR light 1 (CDRL1), CDR light 2 (CDRL2), and CDR light 3 (CDRL3); and three heavy chain CDRs: CDR heavy 1 (CDRH1), CDR heavy 2 (CDRH2) and CDR heavy 3 (CDRH3). In certain embodiments the CDRL1 comprises the amino acid sequence of SEQ ID NO: 13, a variant of SEQ ID NO: 13, a conservatively modified variant of SEQ ID NO: 13, a variant of SEQ ID NO: 13 that comprises the canonical structure class of 3, SEQ ID NO: 15, a variant of SEQ ID NO: 15, a conservatively modified variant of SEQ ID NO: 15, or a variant of SEQ ID NO: 15 that comprises the canonical structure class of 2A; the CDRL2 comprises the amino acid sequence of SEQ ID NO: 16, a variant of SEQ ID NO: 16, a conservatively modified variant of SEQ ID NO: 16, a variant of SEQ ID NO: 16 that comprises the canonical structure class of 1, SEQ ID NO: 18, a variant of SEQ ID NO: 18, a conservatively modified variant of SEQ ID NO: 18, a variant of SEQ ID NO: 18 that comprises the canonical structure class of 1, SEQ ID NO: 19, a variant of SEQ ID NO: 19, a conservatively modified variant of SEQ ID NO: 19, a variant of SEQ ID NO: 19 that comprises the canonical structure class of 1, SEQ ID NO: 20, a variant of SEQ ID NO: 20, a conservatively modified variant of SEQ ID NO: 20, a variant of SEQ ID NO: 20 that comprises the canonical structure class of 1, SEQ ID NO: 21, a variant of SEQ ID NO: 21, a conservatively modified variant of SEQ ID NO: 21, or a variant of SEQ ID NO: 21 that comprises the canonical structure class of 1, the CDRL3 comprises the amino acid sequence of SEQ ID NO: 22, a variant of SEQ ID NO: 22, a conservatively modified variant of SEQ ID NO: 22, or a variant of SEQ ID NO: 22 that comprises the canonical structure class of 1, SEQ ID NO: 24, a variant of SEQ ID NO: 24, a conservatively modified variant of SEQ ID NO: 24, a variant of SEQ ID NO: 24 that comprises the canonical structure class of 1, SEQ ID NO: 25, a variant of SEQ ID NO: 25, a conservatively modified variant of SEQ ID NO: 25, a variant of SEQ ID NO: 25 that comprises the canonical structure class of 1, SEQ ID NO: 26, a variant of SEQ ID NO: 26, a conservatively modified variant of SEQ ID NO: 26, or a variant of SEQ ID NO: 26 that comprises the canonical structure class of 1, the CDRH1 comprises the amino acid sequence of SEQ ID NO: 27, a variant of SEQ ID NO: 27, a conservatively modified variant of SEQ ID NO: 27, a variant of SEQ ID NO: 27 that comprises the canonical structure class of 1, SEQ ID NO: 29, a variant of SEQ ID NO: 29, a conservatively modified variant of SEQ ID NO: 29, a variant of SEQ ID NO: 29 that comprises the canonical structure class of 1, SEQ ID NO: 30, a variant of SEQ ID NO: 30, a conservatively modified variant of SEQ ID NO: 30, or a variant of SEQ ID NO: 30 that comprises the canonical structure class of 1, the CDRH2 comprises the amino acid sequence of SEQ ID NO: 31, a variant of SEQ ID NO: 31, a conservatively modified variant of SEQ ID NO: 31, or a variant of SEQ ID NO: 31 that comprises the canonical structure class of 1, SEQ ID NO: 33, a variant of SEQ ID NO: 33, a conservatively modified variant of SEQ ID NO: 33, a variant of SEQ ID NO: 33 that comprises the canonical structure class of 2A, SEQ ID NO: 34, a variant of SEQ ID NO: 34, a conservatively modified variant of SEQ ID NO: 34, a variant of SEQ ID NO: 34 that comprises the canonical structure class of 1, SEQ ID NO: 35, a variant of SEQ ID NO: 35, a conservatively modified variant of SEQ ID NO: 35, or a variant of SEQ ID NO: 35 that comprises the canonical structure class of 1, the CDRH3 comprises the amino acid sequence of SEQ ID NO: 36, a variant of SEQ ID NO: 36, a conservatively modified variant of SEQ ID NO: 36, a variant of SEQ ID NO: 35 that comprises the canonical structure class of 6, SEQ ID NO: 38, a variant of SEQ ID NO: 38, a conservatively modified variant of SEQ ID NO: 38, a variant of SEQ ID NO: 38 that comprises the canonical structure class of 11, SEQ ID NO: 114, a variant of SEQ ID NO: 114, a conservatively modified variant of SEQ ID NO: 114, or a variant of SEQ ID NO: 114 that comprises the canonical structure class of 11. In particular embodiments the antibody and antigen binding fragment thereof bind canine PD-1 and block the binding of canine PD-1 to canine Programmed Death Ligand 1 (PD-L1). In related embodiments the antibody also blocks the binding of canine PD-1 to canine Programmed Death Ligand 2 (PD-L2). In particular embodiments the isolated mammalian antibody is a caninized antibody. In more particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104.

In other embodiments the CDRL1 comprises the amino acid sequence of SEQ ID NO: 13, a variant of SEQ ID NO: 13, a conservatively modified variant of SEQ ID NO: 13, or a variant of SEQ ID NO: 13 that comprises the canonical structure class of 3; the CDRL2 comprises the amino acid sequence of SEQ ID NO: 16, a variant of SEQ ID NO: 16, a conservatively modified variant of SEQ ID NO: 16, or a variant of SEQ ID NO: 16 that comprises the canonical structure class of 1; the CDRL3 comprises the amino acid sequence of SEQ ID NO: 22, a variant of SEQ ID NO: 22, a conservatively modified variant of SEQ ID NO: 22, or a variant of SEQ ID NO: 22 that comprises the canonical structure class of 1, the CDRH1 comprises the amino acid sequence of SEQ ID NO: 27, a variant of SEQ ID NO: 27, a conservatively modified variant of SEQ ID NO: 27, or a variant of SEQ ID NO: 27 that comprises the canonical structure class of 1; the CDRH2 comprises the amino acid sequence of SEQ ID NO: 31, a variant of SEQ ID NO: 31, a conservatively modified variant of SEQ ID NO: 31, and a variant of SEQ ID NO: 31 that comprises the canonical structure class of 1, the CDRH3 comprises the amino acid sequence of SEQ ID NO: 36, a variant of SEQ ID NO: 36, a conservatively modified variant of SEQ ID NO: 36, or a variant of SEQ ID NO: 36 that comprises the canonical structure class of 6. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104. In more particular embodiments when bound to canine PD-1, the antibody or antigen binding fragment thereof binds to at least one amino acid residue within SEQ ID NO: 102.

In yet other embodiments the CDRL1 comprises the amino acid sequence of SEQ ID NO: 13, a variant of SEQ ID NO: 13, a conservatively modified variant of SEQ ID NO: 13, or a variant of SEQ ID NO: 13 that comprises the canonical structure class of 3; the CDRL2 comprises the amino acid sequence of SEQ ID NO: 19, a variant of SEQ ID NO: 19, a conservatively modified variant of SEQ ID NO: 19, or a variant of SEQ ID NO: 19 that comprises the canonical structure class of 1; the CDRL3 comprises the amino acid sequence of SEQ ID NO: 25, a variant of SEQ ID NO: 25, a conservatively modified variant of SEQ ID NO: 25, or a variant of SEQ ID NO: 25 that comprises the canonical structure class of 1, the CDRH1 comprises the amino acid sequence of SEQ ID NO: 27, a variant of SEQ ID NO: 27, a conservatively modified variant of SEQ ID NO: 27, or a variant of SEQ ID NO: 27 that comprises the canonical structure class of 1; the CDRH2 comprises the amino acid sequence of SEQ ID NO: 31, a variant of SEQ ID NO: 31, a conservatively modified variant of SEQ ID NO: 31, and a variant of SEQ ID NO: 31 that comprises the canonical structure class of 1, the CDRH3 comprises the amino acid sequence of SEQ ID NO: 36, a variant of SEQ ID NO: 36, a conservatively modified variant of SEQ ID NO: 36, or a variant of SEQ ID NO: 36 that comprises the canonical structure class of 6. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104. In more specific embodiments when bound to canine PD-1, the antibodies or antigen binding fragments thereof bind to one or both amino acid residues R₇₅, and R₉₀ of SEQ ID NO: 2

In still other embodiments the CDRL1 comprises the amino acid sequence of SEQ ID NO: 13, a variant of SEQ ID NO: 13, a conservatively modified variant of SEQ ID NO: 13, or a variant of SEQ ID NO: 13 that comprises the canonical structure class of 3; the CDRL2 comprises the amino acid sequence of SEQ ID NO: 20, a variant of SEQ ID NO: 20, a conservatively modified variant of SEQ ID NO: 20, or a variant of SEQ ID NO: 20 that comprises the canonical structure class of 1; the CDRL3 comprises the amino acid sequence of SEQ ID NO: 25, a variant of SEQ ID NO: 25, a conservatively modified variant of SEQ ID NO: 25, or a variant of SEQ ID NO: 25 that comprises the canonical structure class of 1, the CDRH1 comprises the amino acid sequence of SEQ ID NO: 27, a variant of SEQ ID NO: 27, a conservatively modified variant of SEQ ID NO: 27, or a variant of SEQ ID NO: 27 that comprises the canonical structure class of 1; the CDRH2 comprises the amino acid sequence of SEQ ID NO: 34, a variant of SEQ ID NO: 34, a conservatively modified variant of SEQ ID NO: 34, and a variant of SEQ ID NO: 34 that comprises the canonical structure class of 1, the CDRH3 comprises the amino acid sequence of SEQ ID NO: 36, a variant of SEQ ID NO: 36, a conservatively modified variant of SEQ ID NO: 36, or a variant of SEQ ID NO: 36 that comprises the canonical structure class of 6. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104.

In yet other embodiments the CDRL1 comprises the amino acid sequence of SEQ ID NO: 13, a variant of SEQ ID NO: 13, a conservatively modified variant of SEQ ID NO: 13, or a variant of SEQ ID NO: 13 that comprises the canonical structure class of 3; the CDRL2 comprises the amino acid sequence of SEQ ID NO: 16, a variant of SEQ ID NO: 16, a conservatively modified variant of SEQ ID NO: 16, or a variant of SEQ ID NO: 16 that comprises the canonical structure class of 1; the CDRL3 comprises the amino acid sequence of SEQ ID NO: 22, a variant of SEQ ID NO: 22, a conservatively modified variant of SEQ ID NO: 22, or a variant of SEQ ID NO: 22 that comprises the canonical structure class of 1, the CDRH1 comprises the amino acid sequence of SEQ ID NO: 30, a variant of SEQ ID NO: 30, a conservatively modified variant of SEQ ID NO: 30, or a variant of SEQ ID NO: 30 that comprises the canonical structure class of 1; the CDRH2 comprises the amino acid sequence of SEQ ID NO: 31, a variant of SEQ ID NO: 31, a conservatively modified variant of SEQ ID NO: 31, and a variant of SEQ ID NO: 31 that comprises the canonical structure class of 1, the CDRH3 comprises the amino acid sequence of SEQ ID NO: 36, a variant of SEQ ID NO: 36, a conservatively modified variant of SEQ ID NO: 36, or a variant of SEQ ID NO: 36 that comprises the canonical structure class of 6. In particular embodiments when bound to canine PD-1, the antibody binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104.

In still other embodiments the CDRL1 comprises the amino acid sequence of SEQ ID NO: 15, a variant of SEQ ID NO: 15, a conservatively modified variant of SEQ ID NO: 15, or a variant of SEQ ID NO: 15 that comprises the canonical structure class of 2A; the CDRL2 comprises the amino acid sequence of SEQ ID NO: 18, a variant of SEQ ID NO: 18, a conservatively modified variant of SEQ ID NO: 18, or a variant of SEQ ID NO: 18 that comprises the canonical structure class of 1; the CDRL3 comprises the amino acid sequence of SEQ ID NO: 24, a variant of SEQ ID NO: 24, a conservatively modified variant of SEQ ID NO: 24, or a variant of SEQ ID NO: 24 that comprises the canonical structure class of 1, the CDRH1 comprises the amino acid sequence of SEQ ID NO: 29, a variant of SEQ ID NO: 29, a conservatively modified variant of SEQ ID NO: 29, or a variant of SEQ ID NO: 29 that comprises the canonical structure class of 1; the CDRH2 comprises the amino acid sequence of SEQ ID NO: 33, a variant of SEQ ID NO: 33, a conservatively modified variant of SEQ ID NO: 33, and a variant of SEQ ID NO: 33 that comprises the canonical structure class of 1, the CDRH3 comprises the amino acid sequence of SEQ ID NO: 38, a variant of SEQ ID NO: 38, a conservatively modified variant of SEQ ID NO: 38, or a variant of SEQ ID NO: 38 that comprises the canonical structure class of 11. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104. In more particular embodiments when bound to canine PD-1, the antibody or antigen binding fragment thereof binds to at least one amino acid residue within SEQ ID NO: 84.

In yet other embodiments the CDRL1 comprises the amino acid sequence of SEQ ID NO: 15, a variant of SEQ ID NO: 15, a conservatively modified variant of SEQ ID NO: 15, or a variant of SEQ ID NO: 15 that comprises the canonical structure class of 2A; the CDRL2 comprises the amino acid sequence of SEQ ID NO: 21, a variant of SEQ ID NO: 21, a conservatively modified variant of SEQ ID NO: 21, or a variant of SEQ ID NO: 21 that comprises the canonical structure class of 1; the CDRL3 comprises the amino acid sequence of SEQ ID NO: 26, a variant of SEQ ID NO: 26, a conservatively modified variant of SEQ ID NO: 26, or a variant of SEQ ID NO: 26 that comprises the canonical structure class of 1, the CDRH1 comprises the amino acid sequence of SEQ ID NO: 29, a variant of SEQ ID NO: 29, a conservatively modified variant of SEQ ID NO: 29, or a variant of SEQ ID NO: 29 that comprises the canonical structure class of 1; the CDRH2 comprises the amino acid sequence of SEQ ID NO: 35, a variant of SEQ ID NO: 35, a conservatively modified variant of SEQ ID NO: 35, and a variant of SEQ ID NO: 35 that comprises the canonical structure class of 1, the CDRH3 comprises the amino acid sequence of SEQ ID NO: 114, a variant of SEQ ID NO: 114, a conservatively modified variant of SEQ ID NO: 114, or a variant of SEQ ID NO: 114 that comprises the canonical structure class of 11. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104.

In still other embodiments the CDRL1 comprises the amino acid sequence of SEQ ID NO: 14, a variant of SEQ ID NO: 14, a conservatively modified variant of SEQ ID NO: 14, or a variant of SEQ ID NO: 14 that comprises the canonical structure class of 4; the CDRL2 comprises the amino acid sequence of SEQ ID NO: 17, a variant of SEQ ID NO: 17, a conservatively modified variant of SEQ ID NO: 17, or a variant of SEQ ID NO: 17 that comprises the canonical structure class of 1; the CDRL3 comprises the amino acid sequence of SEQ ID NO: 23, a variant of SEQ ID NO: 23, a conservatively modified variant of SEQ ID NO: 23, or a variant of SEQ ID NO: 23 that comprises the canonical structure class of 1, the CDRH1 comprises the amino acid sequence of SEQ ID NO: 28, a variant of SEQ ID NO: 28, a conservatively modified variant of SEQ ID NO: 28, or a variant of SEQ ID NO: 28 that comprises the canonical structure class of 1; the CDRH2 comprises the amino acid sequence of SEQ ID NO: 32, a variant of SEQ ID NO: 32, a conservatively modified variant of SEQ ID NO: 32, and a variant of SEQ ID NO: 32 that comprises the canonical structure class of 2A, the CDRH3 comprises the amino acid sequence of SEQ ID NO: 37, a variant of SEQ ID NO: 37, a conservatively modified variant of SEQ ID NO: 37, or a variant of SEQ ID NO: 37 that comprises the canonical structure class of 13. In particular embodiments when bound to canine PD-1, the antibody or antigen binding fragment thereof binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, SEQ ID NO: 103, and/or SEQ ID NO: 104. In more particular embodiments when bound to canine PD-1, the antibody or antigen binding fragment thereof binds to at least one amino acid residue within SEQ ID NO: 83, SEQ ID NO: 84 and/or SEQ ID NO: 100. In more specific embodiments when bound to canine PD-1, the antibodies or antigen binding fragments thereof bind to one or more amino acid residues of the following arginine residues: R₆₂, R₆₉, R₇₂, and R₇₅ of SEQ ID NO: 2.

The present invention includes antibodies and antigen binding fragments thereof that bind canine Programmed Death Receptor 1 (canine PD-1) with specificity, that when they are bound to canine PD-1, the antibody binds to at least one amino acid residue within SEQ ID NO: 103. In particular embodiments of this type, the antibodies and antigen binding fragments thereof bind canine PD-1 and block the binding of canine PD-1 to canine Programmed Death Ligand 1 (PD-L1). In more particular embodiments the antibodies and antigen binding fragments thereof bind canine PD-1 and also block the binding of canine PD-1 to canine Programmed Death Ligand 2 (PD-L2),

Accordingly, in particular embodiments when bound to canine PD-1, the antibody (including the antibodies with one or more variant CDR, e.g., a variant including a conservatively modified variant and/or a variant that comprises a defined canonical structure class) binds to at least one amino acid residue within one or more amino acid sequences of the following: SEQ ID NO: 83, SEQ ID NO: 84, SEQ ID NO: 99, SEQ ID NO: 100, of SEQ ID NO: 101, SEQ ID NO: 102, and/or SEQ ID NO: 104. In even more particular embodiments when bound to canine PD-1, the antibodies or antigen binding fragments thereof bind to one or more amino acid residues of the following arginine residues: R₆₂, R₆₉, R₇₂, R₇₅, and R₉₀ of SEQ ID NO: 2. In specific embodiments when bound to canine PD-1, the antibodies or antigen binding fragments thereof bind to at least one amino acid residue within SEQ ID NO: 104. In more specific embodiments when bound to canine PD-1, the antibodies or antigen binding fragments thereof bind to one or more amino acid residues of the following arginine residues: R₆₂, R₆₉, R₇₂, and R₇₅ of SEQ ID NO: 2. In even more specific embodiments when bound to canine PD-1, the antibodies or antigen binding fragments thereof bind to R₇₅ of SEQ ID NO: 2.

The present invention further provides mammalian antibodies or antigen binding fragments thereof that bind to canine PD-1 with a dissociation constant (Kd) that is lower (e.g., 1×10⁻¹³ M, or lower) than 1×10⁻¹² M. In particular embodiments the mammalian antibodies or antigen binding fragments thereof bind to canine PD-1 with a dissociation constant of 1×10⁻⁵ M to 1×10⁻¹² M. In more particular embodiments the mammalian antibodies or antigen binding fragments thereof bind to canine PD-1 with a dissociation constant of 1×10⁻⁷ M to 1×10⁻¹¹ M. In still more particular embodiments the mammalian antibodies or antigen binding fragments thereof bind to canine PD-1 with a dissociation constant of 1×10⁻⁸ M to 1×10⁻¹¹ M. In yet more particular embodiments the mammalian antibodies or antigen binding fragments thereof bind to canine PD-1 with a dissociation constant of 1×10⁻⁸M to 1×10⁻¹⁰M.

The present invention also provides mammalian antibodies or antigen binding fragments thereof that bind to canine PD-1 with an on rate (kₒₙ) that is greater than 1×10⁷ M⁻¹ s⁻¹. In particular embodiments the mammalian antibodies or antigen binding fragments thereof bind to canine PD-1 with an on rate of 1×10²M⁻¹ s⁻¹ to 1×10⁷ M⁻¹ s⁻¹. In more particular embodiments the mammalian antibodies or antigen binding fragments thereof bind to canine PD-1 with an on rate of 1×10³ M⁻¹ s⁻¹ to 1×10⁶M⁻¹ s⁻¹. In still more particular embodiments the mammalian antibodies or antigen binding fragments thereof bind to canine PD-1 with an on rate of 1×10³ M⁻¹ s⁻¹ to 1×10⁵M⁻¹ s⁻¹. In yet more particular embodiments the mammalian antibodies or antigen binding fragments thereof bind to canine PD-1 on rate of 1×10⁴ M⁻¹ s⁻¹ to 1×10⁵M⁻¹ s⁻¹.

The present invention further provides mammalian antibodies or antigen binding fragments thereof that bind to canine PD-1 with an off rate (k_{off}) slower than 1×10⁻⁷ s⁻¹. In particular embodiments the mammalian antibodies or antigen binding fragments thereof bind to canine PD-1 with an off rate of 1×10⁻³ s⁻¹ to 1×10⁸ s⁻¹. In more particular embodiments the mammalian antibodies or antigen binding fragments thereof bind to canine PD-1 with an off rate of 1×10⁻⁴ s⁻¹ to 1×10⁻⁷ s⁻¹. In still more particular embodiments the mammalian antibodies or antigen binding fragments thereof bind to canine PD-1 with an off rate of 1×10⁻⁵ s⁻¹ to 1×10⁻⁷ s⁻¹.

In related embodiments, the mammalian antibodies or antigen binding fragments thereof stimulate antigen-specific memory responses to a tumor or pathogen. In particular embodiments, the mammalian antibodies or antigen binding fragments thereof stimulate an antibody response in vivo. In other particular embodiments, the mammalian antibodies or antigen binding fragments thereof stimulate an immune response in an animal subject. In more specific embodiments the animal subject is a canine. In a related embodiment, the animal subject is a feline.

Accordingly, any of the antibodies of the present invention can exhibit one, two, three, four, five, or all these properties, i.e., the aforesaid dissociation constants with canine PD-1, the aforesaid on rates for binding with canine PD-1, the aforesaid off rates for dissociating from from the antibody-canine PD-1 binding complex, stimulating an antigen-specific memory responses to a tumor or pathogen, stimulating an antibody response in vivo, and/or stimulating an immune response in an animal subject

As indicated above, the antibodies (and antigen binding fragments thereof) of the present invention, including the aforesaid antibodies (and antigen binding fragments thereof), can be monoclonal antibodies (and antigen binding fragments thereof), mammalian antibodies (and antigen binding fragments thereof), e.g., murine (mouse) antibodies (and antigen binding fragments thereof), caninized antibodies (and antigen binding fragments thereof) including caninized murine antibodies (and antigen binding fragments thereof), and in certain embodiments the antibodies (and antigen binding fragments thereof) are isolated.

The present invention further provides nucleic acids (including isolated nucleic acids) that encode any one of the light chains of the caninized antibody of the present invention. Similarly, the present invention provides isolated nucleic acids that encode any one of the heavy chains of the caninized antibody of the present invention. Examples of specific nucleotide sequences are provided herein.

The present invention further provides expression vectors that comprise one or more of the nucleic acids (including isolated nucleic acids) of the present invention. The present invention further provides host cells that comprise one or more expression vectors of the present invention.

In particular embodiments, the antibody is a recombinant antibody or an antigen binding fragment thereof. In related embodiments, the variable heavy chain domain and variable light chain domain are connected by a flexible linker to form a single-chain antibody.

In particular embodiments, the antibody or antigen binding fragment is a Fab fragment. In other embodiments, the the antibody or antigen binding fragment is a Fab′ fragment. In other embodiments, the antibody or antigen binding fragment is a (Fab′)₂ fragment. In still other embodiments, the antibody or antigen binding fragment is a diabody. In particular embodiments, the antibody or antigen binding fragment is a domain antibody. In particular embodiments, the antibody or antigen binding fragment is a camelized single domain antibody.

In particular embodiments, a caninized murine anti-canine PD-1 antibody or antigen binding fragment increases the immune response of the canine subject being treated.

The present invention further provides isolated nucleic acids that encode caninized murine anti-canine PD-1 antibodies or portions thereof. In related embodiments such antibodies or antigen binding fragments can be used for the preparation of a medicament to treat cancer in a canine subject. Alternatively, or in conjunction, the present invention provides for the use of any of the antibodies or antibody fragments of the present invention for diagnostic use. In yet additional embodiments, a kit is provided comprising any of the caninized antibodies or antigen binding fragments disclosed herein.

In yet additional embodiments, an expression vector is provided comprising an isolated nucleic acid encoding any of the caninized murine anti-canine PD-1 antibodies or antigen binding fragments of the invention. The invention also relates to a host cell comprising any of the expression vectors described herein. In particular embodiments, these nucleic acids, expression vectors or polypeptides of the invention are useful in methods of making an antibody.

The present invention further provides antigenic peptides (including isolated antigenic peptides) that consist of 80 or fewer amino acid residues that comprise the amino acid sequence of SEQ ID NO: 103, and/or SEQ ID NO: 83, and/or SEQ ID NO: 84, and/or SEQ ID NO: 99, and/or SEQ ID NO: 100, and/or SEQ ID NO: 101, and/or SEQ ID NO: 102, and/or SEQ ID NO: 104. In related embodiments, the antigenic peptides (including isolated peptides) consist of 60 or fewer amino acid residues that comprise the amino acid sequence of SEQ ID NO: 103, and/or SEQ ID NO: 83, and/or SEQ ID NO: 84, and/or SEQ ID NO: 99, and/or SEQ ID NO: 100, and/or SEQ ID NO: 101, and/or SEQ ID NO: 102, and/or SEQ ID NO: 104. In other embodiments the antigenic peptides consist of 10 to 44 amino acid residues from the amino acid sequence of SEQ ID NO: 103. In still other embodiments the antigenic peptides consist of 15 to 45 amino acid residues from the amino acid sequence of SEQ ID NO: 103.

The present invention further provides antigenic peptides (including isolated peptides) that consist of 80 or fewer amino acid residues that comprise an amino acid sequence that is 80%, 85%, 90%, 95% or 100% identical with SEQ ID NO: 103, and/or SEQ ID NO: 83, and/or SEQ ID NO: 84, and/or SEQ ID NO: 99, and/or SEQ ID NO: 100, and/or SEQ ID NO: 101, and/or SEQ ID NO: 102, and/or SEQ ID NO: 104 and binds to an isolated mammalian antibody or antigen binding fragment thereof of the present invention. In related embodiments, the antigenic peptides (including isolated antigenic peptides) consist of 60 or fewer amino acid residues that comprise an amino acid sequence that is 80%, 85%, 90%, 95% or 100% identical with SEQ ID NO: 103 and/or SEQ ID NO: 83, and/or SEQ ID NO: 84, and/or SEQ ID NO: 9, and/or SEQ ID NO: 100, and/or SEQ ID NO: 101, and/or SEQ ID NO: 102, and/or SEQ ID NO: 104 and binds to an isolated mammalian antibody or antigen binding fragment thereof. In other embodiments the peptides consist of 10 to 44 amino acid residues from an amino acid sequence that is 80%, 85%, 90%, 95% or 100% identical with SEQ ID NO: 103 and/or SEQ ID NO: 83, and/or SEQ ID NO: 84, and/or SEQ ID NO: 99, and/or SEQ ID NO: 100, and/or SEQ ID NO: 101, and/or SEQ ID NO: 102, and/or SEQ ID NO: 104 and binds to an isolated mammalian antibody or antigen binding fragment thereof. In particular embodiments the antibody is IBS. In other embodiments the antibody is 3B6. In other particular embodiments the antibody is 2H9. In still other embodiments, the antibody is 2G9. In yet other embodiments the antibody is 1A1. In still other embodiments, the antibody is 1E4.

The present invention further provides fusion proteins that comprise any of the aforesaid antigenic peptides. In a particular embodiment, the fusion protein comprises such an antigenic peptide and an Fc region of a non-canine mammalian IgG antibody. In a more particular embodiment the fusion protein comprises an Fc region of a non-canine mammalian IgG antibody. In certain embodiments the non-canine mammalian IgG antibody is a murine IgG. In alternative embodiments the non-canine mammalian IgG antibody is a human IgG. In other embodiments the non-canine mammalian IgG antibody is an equine IgG. In still other embodiments the non-canine mammalian IgG antibody is a porcine IgG. In yet other embodiments the non-canine mammalian IgG antibody is a bovine IgG.

In particular embodiments the non-canine mammalian IgG antibody is an IgG1. In other embodiments the non-canine mammalian IgG antibody is an IgG2a. In still other embodiments the non-canine mammalian IgG antibody is an IgG3. In yet other embodiments the non-canine mammalian IgG antibody is an IgG4.

In other embodiments the fusion protein comprises any of the aforesaid antigenic peptides and maltose-binding protein. In yet other embodiments, the fusion protein comprises any of the aforesaid antigenic peptides and beta-galactosidase. In still other embodiments the fusion protein comprises any of the aforesaid antigenic peptides and glutathione S-transferase. In yet other embodiments, the fusion protein comprises any of the aforesaid antigenic peptides and thioredoxin. In still other embodiments the fusion protein comprises any of the aforesaid antigenic peptides and Gro EL. In yet other embodiments the fusion protein comprises any of the aforesaid antigenic peptides and NusA.

The present invention further provides nucleic acids (including isolated nucleic acids) that encode the antigenic peptides and the corresponding fusion proteins of the present invention. The present invention also provides expression vectors that comprise these nucleic acids.

In addition, the present invention includes pharmaceutical compositions comprising anti-canine PD-1 antibodies or antigen binding fragments thereof of the present invention, antigenic peptides (including isolated antigenic peptides) from canine PD-1, fusion proteins comprising the antigenic peptides from canine PD-1 of the present invention, nucleic acids (including isolated nucleic acids) encoding the antigenic fragments and/or fusion proteins of the present invention, the expression vectors comprising such nucleic acids, or any combination thereof, and a pharmaceutically acceptable carrier or diluent.

In addition, the present invention provides methods of increasing the activity of an immune cell, comprising administering to a subject in need thereof a therapeutically effective amount of such pharmaceutical compositions. In certain embodiments the method is used for the treatment of cancer. In other embodiments, the method is used in the treatment of an infection or infectious disease. In still other embodiments, a caninized antibody of the present invention or antigen binding fragment thereof is used as a vaccine adjuvant.

These and other aspects of the present invention will be better appreciated by reference to the following Brief Description of the Drawings and the Detailed Description.

### BRIEF DESCRIPTION OF THE DRAWINGS

FIG. 1 shows the reactivity of mouse mAbs against extracellular domain of canine PD-1. Various mouse mAbs were tested for their binding to extracellular domain of canine PD-1 by ELISA. Tested mAbs are designated as ⋄ 3B6, ▪ 5F3, −5G5, ×4D12, * 2H9, ● 2C12, + 2G9, ▴ unrelated mAb.

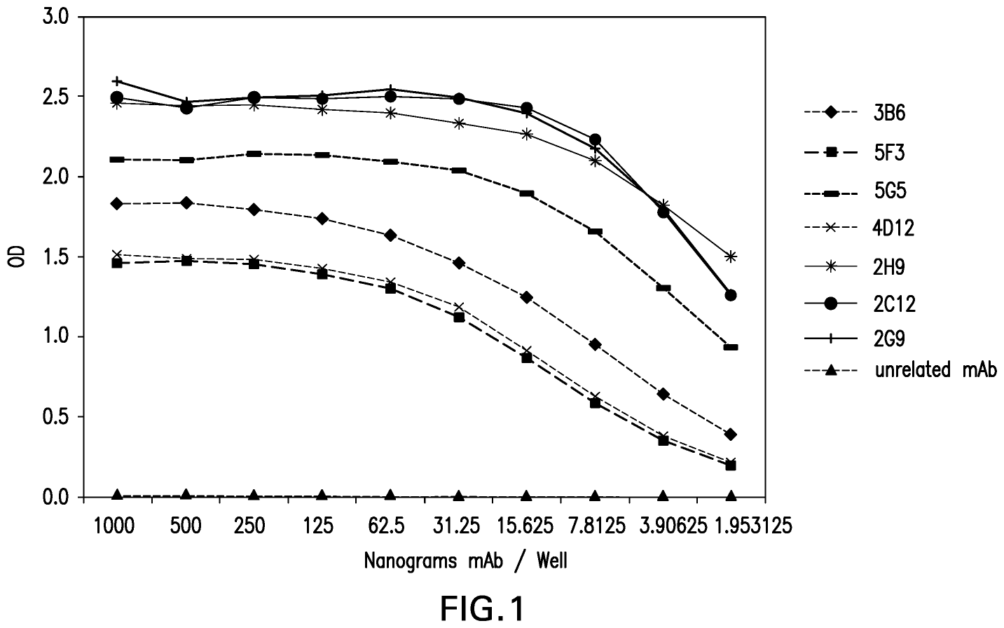

FIG. 2 shows the reactivity of mouse mAbs against cell surface-expressed canine PD-1. Various mouse mAbs were tested for their binding to canine PD-1 expressed on CHO cells by CELISA. Antibodies are designated as: [symbol] GnRh 80 ug/mL; [symbol] 7C9 80 ug/mL; [symbol] 4D12 80 ug/mL; [symbol] 1B580 ug/mL; [symbol] 5F3 80 ug/L and [symbol] 386 30 ug/mL.

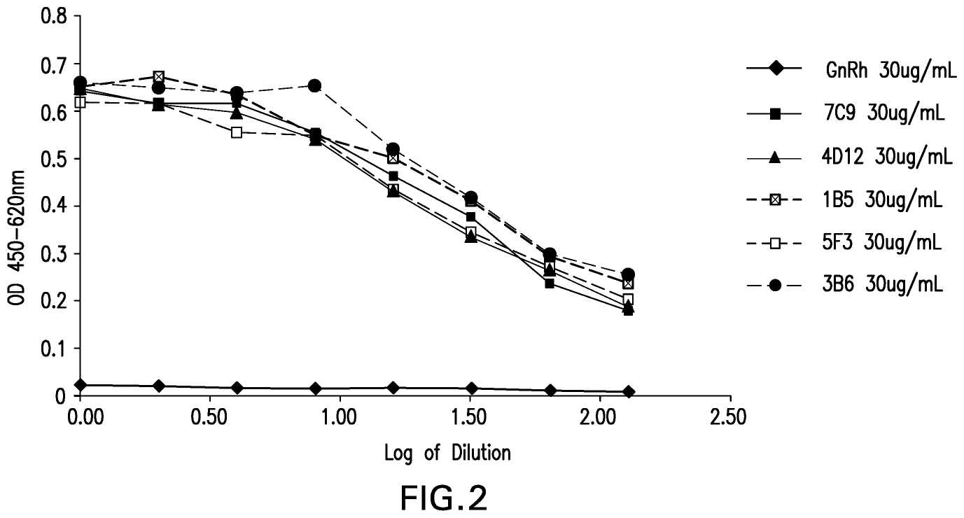

FIG. 3A shows ligand blockade with mouse mAbs against canine PD-1. Various mouse mAbs were tested for their ability to inhibit binding of PD-1 expressed on CHO cells to PD-L1. Antibodies are designated as: [symbol] GnRh 80 ug/mL; [symbol] 386 80 ug/mL; [symbol] 1A1 30 ug/mL; [symbol] 6A8 30 ug/mL and [symbol] 4D12 80 ug/mL.

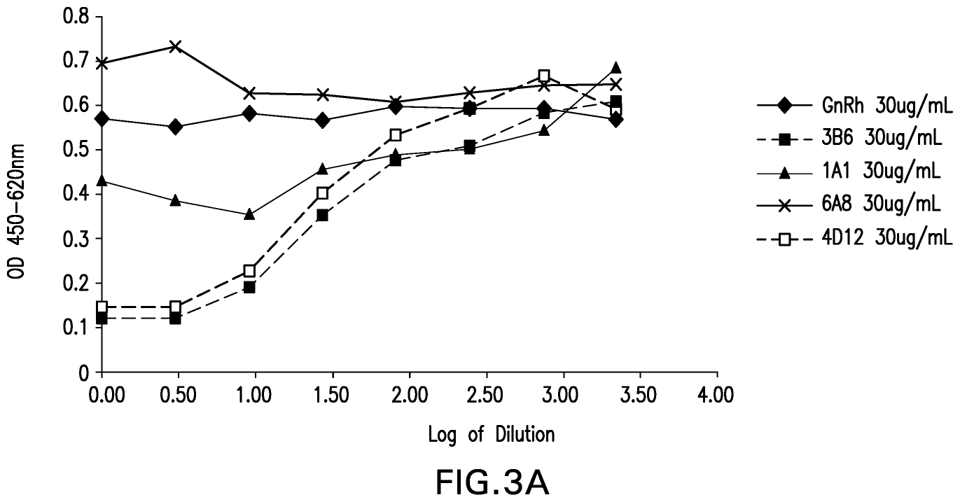

FIG. 3B shows ligand blockade with mouse mAbs against canine PD-1. Various mouse mAbs were tested for their ability to inhibit binding of PD-1 expressed on CHO cells to PD-L1. Antibodies are designated as: [symbol] GnRh 30 ug/mL; [symbol] 3B6 80 g/mL; [symbol] 1H4 63.4 ug/mL; [symbol] 2C12 52.4 ug/mL; [symbol] 2F8 38.2 ug/mL and [symbol] 2G9 54.8 ug/mL.

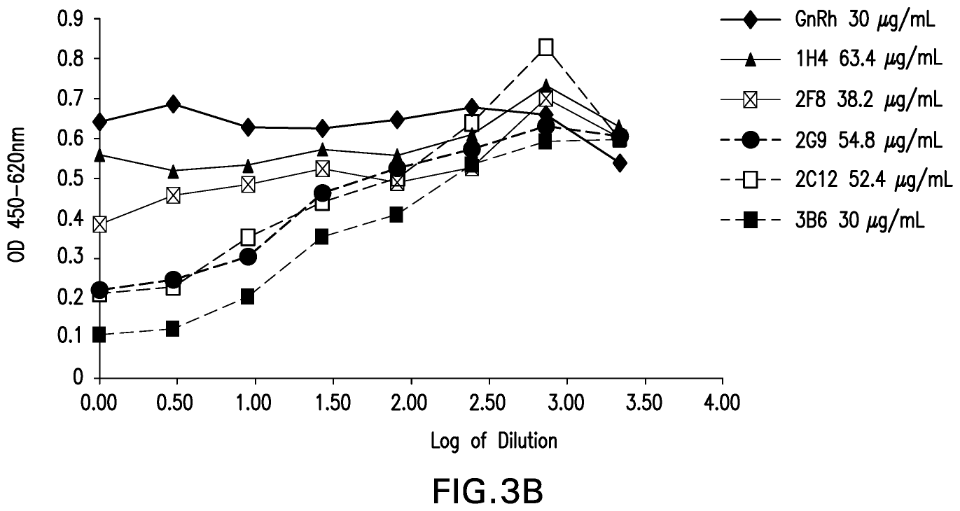

FIG. 3C shows ligand blockade with mouse mAbs against canine PD-1. Various mouse mAbs were tested for their ability to inhibit binding of PD-1 expressed on CHO cells to PD-L1. Antibodies are designated as: [symbol] GnRh 30 ug/mL; [symbol] 386 30 ug/mL; [symbol] and [symbol] .

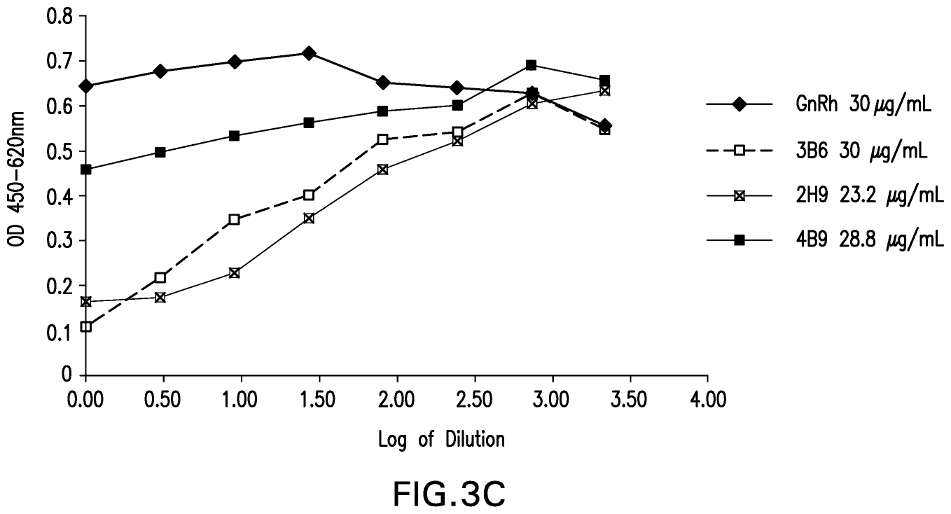

FIG. 4 shows the binding of mouse mAbs to canine PD-1 on CD⁺ T cells in PBMC from healthy dogs. Various mouse mAbs were tested for their ability to bind to canine PD-1 expressed on CD⁺ T cells from PBMC from healthy dogs. Antibodies were tested at 2 fold dilutions covering starting with 0.156-20 μg/ml range.

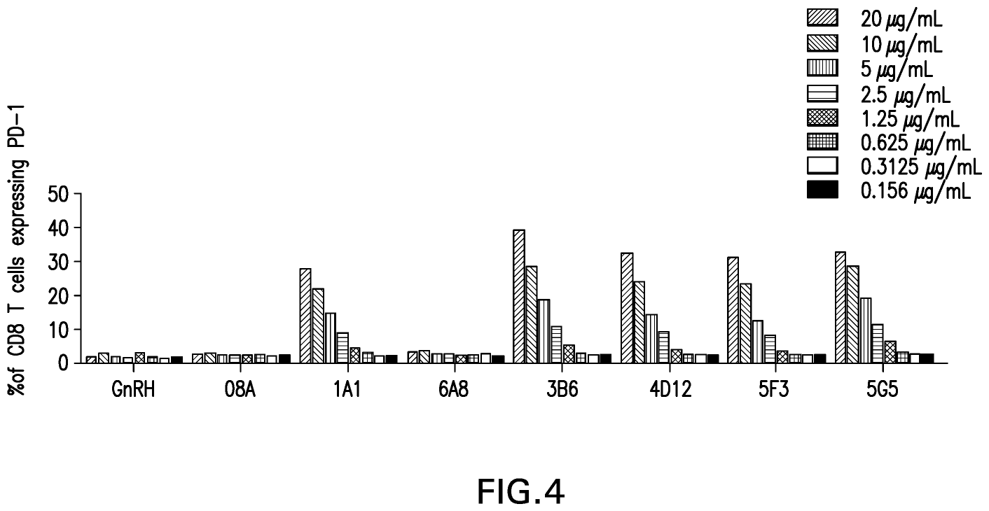

FIG. 5 shows the binding of mouse mAbs to canine PD-1 on CD8⁺ T cells in PBMC from dogs with cancer. Indicated mouse mAbs were tested for their ability to bind to canine PD-1 expressed on CD8⁺ T cells from dogs with cancer (sarcoma). Antibodies were tested at 2.5 and 5 μg/ml.

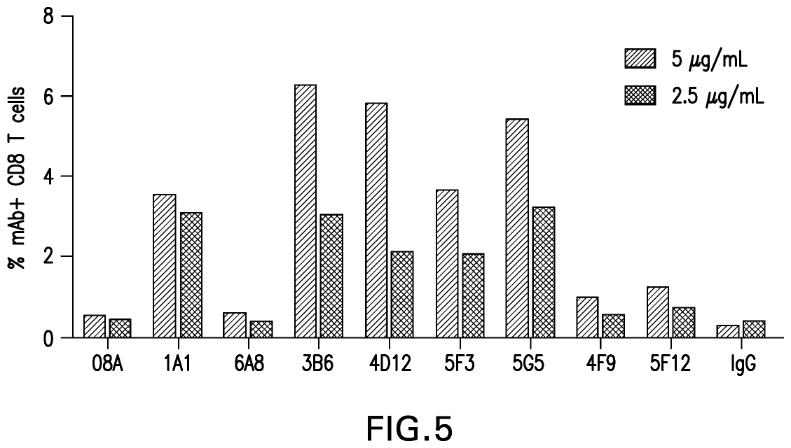

FIG. 6 shows the cytokine secretion induced by mouse mAbs to canine PD-1. Various mouse mAbs were tested for their ability to induce cytokine secretion from PBMC from healthy dogs.

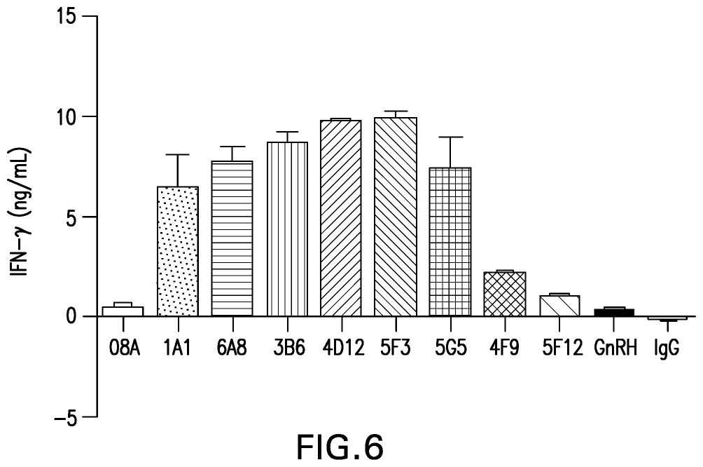

FIG. 7 shows the cytokine secretion induced by mouse mAbs to canine PD-1. Various mouse mAbs were tested for their ability to induce cytokine secretion from PBMC from dogs with cancer (hemangiosarcoma).

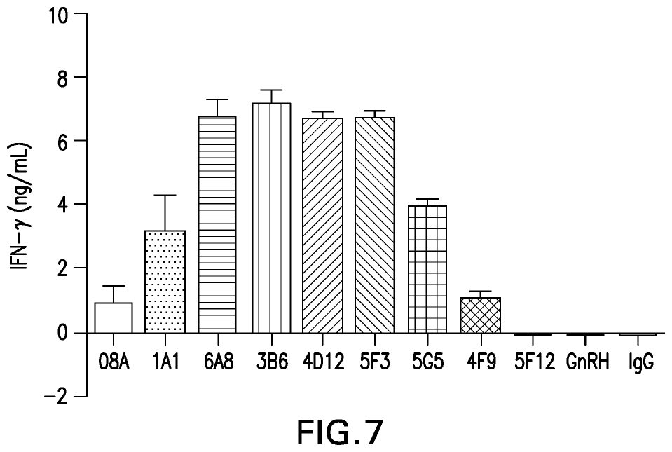

FIGS. 8A-8B provide the alignment of canine IgGB constant heavy chains (CHs) lacking ADCC function. The canine wild type IgB [cIgGB wt], Canine IgGB(+)A-hinge [cIgGB(+) A-hinge], Canine IgGB(+) D-hinge [cIgGB(+) D-hinge], and Canine IgGB (−) ADCC [cIgGB(−) ADCC] are depicted. The (+) A-hinge is the replacement with IgG-A hinge plus a lysine and asparagine amino acid replacement as shown; the (+) D-hinge is the replacement with IgG-D hinge plus a lysine and the asparagine amino acid replacement as shown. The (−)ADCC is the aspartic acid and asparagine amino acid replacement.

FIG. 9A shows the characterization of the interface between canine PD-1 and the caninized antibody 2G9. The amino acid positions are with respect to the PD-1 amino acid sequence without the signal sequence, i.e., SEQ ID NO: 2. The determination was performed by chemical cross-linking, High-Mass MALDI mass spectrometry and nLC-Orbitrap mass spectrometry.

FIG. 9B shows the characterization of the interface between canine PD-1 and the caninized antibody 3B6. The amino acid positions are with respect to the PD-1 amino acid sequence without the signal sequence, i.e., SEQ ID NO: 2. The determination was performed by chemical cross-linking, High-Mass MALDI mass spectrometry and nLC-Orbitrap mass spectrometry.

### DETAILED DESCRIPTION

### Abbreviations

Throughout the detailed description and examples of the invention the following abbreviations will be used:
ADCC Antibody-dependent cellular cytotoxicity
CDC Complement-dependent cytotoxicity
CDR Complementarity determining region in the immunoglobulin variable regions, defined using the Kabat numbering system
CHO Chinese hamster ovary
EC50 concentration resulting in 50% efficacy or binding
ELISA Enzyme-linked immunosorbant assay
FR Antibody framework region: the immunoglobulin variable regions excluding the CDR regions.
HRP Horseradish peroxidase
IFN interferon
IC50 concentration resulting in 50% inhibition
IgG Immunoglobulin G
Kabat An immunoglobulin alignment and numbering system pioneered by
Elvin A. Kabat [Sequences of Proteins of Immunological Interest, 5th
Ed. Public Health Service, National Institutes of Health, Bethesda, Md. (1991)]
mAb Monoclonal antibody (also Mab or MAb)
MES 2-(N-morpholino)ethanesulfonic acid
MOA Mechanism of action
NHS Normal human serum
PCR Polymerase chain reaction
PK Pharmacokinetics
SEB Staphylococcus Enterotoxin B
TT Tetanus toxoid
V region The segment of IgG chains which is variable in sequence between
different antibodies. It extends to Kabat residue 109 in the light chain and 113
in the heavy chain.
VH Immunoglobulin heavy chain variable region
VK Immunoglobulin kappa light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activation” as it applies to cells or to receptors refers to the activation or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, e.g., a canine experimental subject, cell, tissue, organ, or biological fluid, refers to contact of an exogenous pharmaceutical, therapeutic, diagnostic agent, or composition to the animal e.g., a canine subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., canine, feline, or human) and most preferably a canine.

As used herein, a “substitution of an amino acid residue” with another amino acid residue in an amino acid sequence of an antibody for example, is equivalent to “replacing an amino acid residue” with another amino acid residue and denotes that a particular amino acid residue at a specific position in the amino acid sequence has been replaced by (or substituted for) by a different amino acid residue. Such substitutions can be particularly designed i.e., purposefully replacing an alanine with a serine at a specific position in the amino acid sequence by e.g., recombinant DNA technology. Alternatively, a particular amino acid residue or string of amino acid residues of an antibody can be replaced by one or more amino acid residues through more natural selection processes e.g., based on the ability of the antibody produced by a cell to bind to a given region on that antigen, e.g., one containing an epitope or a portion thereof, and/or for the antibody to comprise a particular CDR that retains the same canonical structure as the CDR it is replacing. Such substitutions/replacements can lead to “variant” CDRs and/or variant antibodies.

“Treat” or “treating” means to administer a therapeutic agent, such as a composition containing any of the antibodies or antigen binding fragments of the present invention, internally or externally to a canine subject or patient having one or more disease symptoms, or being suspected of having a disease, for which the agent has therapeutic activity.

Typically, the agent is administered in an amount effective to alleviate and/or ameliorate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient (e.g., canine), and the ability of the pharmaceutical composition to elicit a desired response in the subject. Whether a disease symptom has been alleviated or ameliorated can be assessed by any clinical measurement typically used by veterinarians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary (e.g., canine) or research subject, refers to therapeutic treatment, as well as research and diagnostic applications. “Treatment” as it applies to a human, veterinary (e.g., canine), or research subject, or cell, tissue, or organ, encompasses contact of the antibodies or antigen binding fragments of the present invention to a canine or other animal subject, a cell, tissue, physiological compartment, or physiological fluid.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, such as any member of the subfamilies Felinae, e.g., cats, lions, tigers, pumas, jaguars, leopards, snow leopards, panthers, North American mountain lions, cheetahs, lynx, bobcats, caracals or any cross breeds thereof. Cats also include domestic cats, pure-bred and/or mongrel companion cats, show cats, laboratory cats, cloned cats, and wild or feral cats.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse antibody) in both chains. Optionally the heavy and/or light chains of the canine antibody may contain some foreign non-CDR residues, e.g., so as to preserve the conformation of the foreign CDRs within the canine antibody, and/or to modify the Fc function, as exemplified below.

Canine PD-1 has been found to comprise the amino acid sequence of SEQ ID NO: 2. In a specific embodiment canine PD-1 is encoded by a nucleic acid that comprises the nucleotide sequence of SEQ ID NO: 1. Canine PD-1 sequences may differ by having, for example, conserved variations in non-conserved regions, but the canine PD-1 will have substantially the same biological function as the canine PD-1 comprising the amino acid sequence of SEQ ID NO: 2. For example, a biological function of PD-1 is to attenuate T-cell responses when bound to PD-L1 and/or PD-L2. That is, PD-1 may be considered a negative regulator. Notably, the cytoplasmic tail of PD-1 contains two tyrosine-based signaling motifs, an ITIM (immunoreceptor tyrosine-based inhibition motif) and an ITSM (immunoreceptor tyrosine-based switch motif). In addition, a biological function of canine PD-1 may be having, for example, an epitope in the extracellular domain that is specifically bound by an antibody of the instant disclosure.

Canine PD-L1 has been found to comprise the amino acid sequence of SEQ ID NO: 8. In a specific embodiment canine PD-L1 is encoded by a nucleotide sequence comprising SEQ ID NO: 7. Canine PD-L1 sequences may differ by having, for example, conserved variations in non-conserved regions, but the canine PD-L1 will have substantially the same biological function as the canine PD-L1 comprising the amino acid sequence of SEQ ID NO: 8. For example, one biological function of PD-L1 is to attenuate T-cell responses when bound to PD-1.

A particular canine PD-1 or PD-L1 amino acid sequence respectively, will generally be at least 90% identical to the canine PD-1 comprising the amino acid sequence of SEQ ID NO: 2, or canine PD-L1 comprising the amino acid sequence of SEQ ID NO: 8, respectively. In certain cases, a canine PD-1 or PD-L1 respectively, may be at least 95%, or even at least 96%, 97%, 98% or 99% identical to the canine PD-1 comprising the amino acid sequence of SEQ ID NO: 2, or the canine PD-L1 comprising the amino acid sequence of SEQ ID NO: 8, respectively. In certain embodiments, a canine PD-1 or a PD-L1 amino acid sequence respectively, will display no more than 10 amino acid differences from the canine PD-1 comprising the amino acid sequence of SEQ ID NO: 2, or the canine PD-L1 comprising the amino acid sequence of SEQ ID NO: 8, respectively. In certain embodiments, the canine PD-1 or the PD-L1 amino acid sequence respectively, may display no more than 5, or even no more than 4, 3, 2, or 1 amino acid difference from the canine PD-1 comprising the amino acid sequence of SEQ ID NO: 2, or the canine PD-L1 comprising the amino acid sequence of SEQ ID NO: 8, respectively. Percent identity can be determined as described herein below.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the mammalian body (e.g., canine body) of cancerous cells, cells or tissues infected with pathogens, or invading pathogens.

Anti-Canine PD-1 Antibodies

The present invention provides isolated antibodies (particularly murine anti-canine PD-1 antibodies and caninized antibodies thereof) or antigen binding fragments thereof that bind canine PD-1 and uses of such antibodies or fragments thereof. In specific embodiments murine anti-canine PD-1 CDRs from murine anti-canine PD-1 antibodies are provided that have been shown to both bind canine PD-1 and to block the binding of canine PD-1 to its ligand, canine PD-L1. These CDRs can be inserted into a modified canine frame of a canine antibody to generate a caninized murine anti-canine PD-1 antibody.

As used herein, an “anti-canine PD-1 antibody” refers to an antibody that was raised against canine PD-1 (e.g., in a mammal such as a mouse or rabbit) and that specifically binds to canine PD-1. An antibody that “specifically binds to canine PD-1,” and in particular canine PD-1, or an antibody that “specifically binds to a polypeptide comprising the amino acid sequence of canine PD-1”, is an antibody that exhibits preferential binding to canine PD-1 as compared to other antigens, but this specificity does not require absolute binding specificity.

An anti-canine PD-1 antibody is considered “specific” for canine PD-1 if its binding is determinative of the presence of canine PD-1 in a sample, or if it is capable of altering the activity of canine PD-1 without unduly interfering with the activity of other molecules in a canine sample, e.g. without producing undesired results such as false positives in a diagnostic context or side effects in a therapeutic context. The degree of specificity necessary for an anti-canine PD-1 antibody may depend on the intended use of the antibody, and at any rate is defined by its suitability for use for an intended purpose. The antibody, or binding compound derived from the antigen-binding site of an antibody, of the contemplated method binds to its antigen, or a variant or mutein thereof, with an affinity that is at least two-fold greater, preferably at least ten-times greater, more preferably at least 20-times greater, and most preferably at least 100-times greater than the affinity with any other antigen.

As used herein, an antibody is said to bind specifically to a polypeptide comprising a given antigen sequence (in this case a portion of the amino acid sequence of canine PD-1) if it binds to polypeptides comprising the portion of the amino acid sequence of canine PD-1, but does not bind to other canine proteins lacking that portion of the sequence of canine PD-1. For example, an antibody that specifically binds to a polypeptide comprising canine PD-1 may bind to a FLAG®-tagged form of canine PD-1, but will not bind to other FLAG®-tagged canine proteins. An antibody, or binding compound derived from the antigen-binding site of an antibody, binds to its canine antigen, or a variant or mutein thereof, “with specificity” when it has an affinity for that canine antigen or a variant or mutein thereof which is at least ten-times greater, more preferably at least 20-times greater, and even more preferably at least 100-times greater than its affinity for any other canine antigen tested.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), canonized antibodies, fully canine antibodies, chimeric antibodies and camelized single domain antibodies. “Parental antibodies” are antibodies obtained by exposure of an immune system to an antigen prior to modification of the antibodies for an intended use, such as caninization of an antibody for use as a canine therapeutic antibody.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antigen binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

A “fragment crystallizable” (“Fc”) region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. [See, Pluckthun, THE PHARMACOLOGY OF MONOCLONAL ANTIBODIES, vol. 113 Rosenburg and Moore eds., Springer-Verlag, New York, pp. 269-315 (1994); WO 88/01649; and U.S. Pat. Nos. 4,946,778 and 5,260,203.]

As used herein, the term “canonical structure” refers to the local conformation that can be adopted by each of the hypervariable regions of the heavy and light chain of an antibody within the framework that they reside. For each hypervariable region, there are a small number of canonical structures (generally denoted by simple integers such as 1 or 2 etc.), which can be predicted with great accuracy from the amino acid sequences of the corresponding hypervariable region (particularly within the context of the amino acid sequence of its framework, as provided below for the corresponding anti-canine PD-1 variable domains). These canonical structures can be determinative regarding whether a modification of the amino acid sequence of a given CDR will result in the retention or loss of the ability to bind to its antigen binding partner [See, Chothia and Lesk, Canonical Structures for the hypervariable regions of immunoglobulins, J. Mol. Biol. 196:901-917(1987); Chothia et al., Conformation of immunoglobulin hypervaribale regions, Nature, 34:877-883(1989); and Al-Lazikani et al., Standard Conformations for the canonical structures of immunoglobulins, J. Mol. Biol. 273:927-948 (1997)].

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. [See, e.g., Muyldermans et al., Trends Biochem. Sci. 26:230 (2001); Reichmann et al., J. Immunol. Methods 231:25 (1999); WO 94/04678; WO 94/25591; U.S. Pat. No. 6,005,079]. In one embodiment, the present invention provides single domain antibodies comprising two VH domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. [See, EP 0 404 097 B1; WO 93/11161; and Holliger et al., Proc. Natl. Acad. Sci. USA 90: 6444-6448 (1993)]. For a review of engineered antibody variants [generally see Holliger and Hudson Nat. Biotechnol. 23:1126-1136 (2005)].

Typically, an antibody or antigen binding fragment of the invention retains at least 10% of its canine PD-1 binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine PD-1 binding affinity as the parental antibody. It is also intended that an an antibody or antigen binding fragment of the invention can include conservative or non-conservative amino acid substitutions (referred to as “conservative variants” or “function conserved variants” of the antibody) that do not substantially alter its biologic activity.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein. As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81: 6851-6855 (1984)]. Typically the variable domains are obtained from an antibody from an experimental animal (the “parental antibody”), such as a rodent, and the constant domain sequences are obtained from the animal subject antibodies, e.g., human or canine so that the resulting chimeric antibody will be less likely to elicit an adverse immune response in a canine or human subject respectively, than the parental (e.g., rodent) antibody.

As used herein, the term “caninized antibody” refers to forms of antibodies that contain sequences from both canine and non-canine (e.g., murine) antibodies. In general, the caninized antibody will comprise substantially all of at least one or more typically, two variable domains in which all or substantially all of the hypervariable loops correspond to those of a non-canine immunoglobulin (e.g., comprising 6 murine anti-canine PD-1 CDRs as exemplified below), and all or substantially all of the framework (FR) regions (and typically all or substantially all of the remaining frame) are those of a canine immunoglobulin sequence. As exemplified herein, a caninized antibody comprises both the three heavy chain CDRs and the three light chain CDRS from a murine anti-canine PD-1 antibody together with a canine frame or a modified canine frame. A modified canine frame comprises one or more amino acids changes as exemplified herein that further optimize the effectiveness of the caninized antibody, e.g., to increase its binding to canine PD-1 and/or its ability to block the binding of canine PD-1 to canine PD-L1.

The term “fully canine antibody” refers to an antibody that comprises canine immunoglobulin protein sequences only. A fully canine antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully canine antibody may contain rat carbohydrate chains if produced in a rat, in a rat cell, or in a hybridoma derived from a rat cell. Similarly, “rat antibody” refers to an antibody that comprises rat immunoglobulin sequences only.

There are four known IgG heavy chain subtypes of dog IgG and they are referred to as IgG-A, IgG-B, IgG-C, and IgG-D. The two known light chain subtypes are referred to as lambda and kappa.

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). [See Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196: 901-917 (1987) defining the CDR regions of an antibody by structure]. As used herein, the term “framework” or “FR” residues refers to those variable domain residues other than the hypervariable region residues defined herein as CDR residues.

Besides binding and activating of canine immune cells, a canine or caninized antibody against PD-1 optimally has two attributes:
1. Lack of effector functions such as antibody-dependent cytotoxicity (ADCC) and complement-dependent cytotoxicity (CDC), and
2. be readily purified on a large scale using industry standard technologies such as that based on protein A chromatography.

None of the naturally occurring canine IgG isotypes satisfy both criteria. For example, IgG-B can be purified using protein A, but has high level of ADCC activity. On the other hand, IgG-A binds weakly to protein A, but displays undesirable ADCC activity. Moreover, neither IgG-C nor IgG-D can be purified on protein A columns, although IgG-D display no ADCC activity. (IgG-C has considerable ADCC activity). The present invention overcomes this difficulty by providing mutant canine IgG-B antibodies specific to PD-1; such antibodies lack effector functions such as ADCC and can be easily of purified using industry standard protein A chromatography.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context. As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. [Nucleic Acids Res. 33:D256-D261 (2005)].

### Properties of Murine Anti-Canine PD-1 and Caninized Murine Anti-Canine PD-1 Antibodies

The present invention provides isolated murine anti-canine PD-1 antibodies and caninized antibodies thereof, methods of use of the antibodies or antigen binding fragments thereof in the treatment of disease e.g., the treatment of cancer in canines. In canine, there are four IgG heavy chains referred to as A, B, C, and D. These heavy chains represent four different subclasses of dog IgG, which are referred to as IgGA, IgGB, IgGC and IgGD. The DNA and amino acid sequences of these four heavy chains were first identified by Tang et al. [Vet. Immunol. Immunopathol. 80: 259-270 (2001)]. The amino acid and DNA sequences for these heavy chains are also available from the GenBank data bases. For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example the kappa light chain amino acid sequence has accession number ABY 57289.1 and the lambda light chain has accession number ABY 55569.1. In the present invention, the amino acid sequence for each of the four canine IgG Fc fragments is based on the identified boundary of CH1 and CH2 domains as determined by Tang et al, supra. Caninized murine anti-canine PD-1 antibodies that bind canine PD-1 include, but are not limited to: antibodies that comprise canine IgG-A, IgG-B, and IgG-D heavy chains and/or canine kappa light chains together with murine anti-canine PD-1 CDRs. Accordingly, the present invention provides isolated murine anti-canine PD-1 and/or caninized murine anti-canine PD-1 antibodies or antigen binding fragments thereof that bind to canine PD-1 and block the binding of canine PD-1 to canine PD-L1.

The present invention further provides full length canine heavy chains that can be matched with corresponding light chains to make a caninized antibody. Accordingly, the present invention further provides caninized murine anti-canine antigen antibodies (including isolated caninized murine anti-canine PD-1 antibodies) and methods of use of the antibodies or antigen binding fragments thereof in the treatment of disease e.g., the treatment of cancer in canines.

The isolated antibody or antigen binding fragment thereof that binds canine PD-1 can comprise one, two, three, four, five, or six of the complementarity determining regions (CDRs) of the murine anti-canine antibody as described herein. The one, two, three, four, five, or six CDRs may be independently selected from the CDR sequences of those provided below. In a further embodiment, the isolated antibody or antigen-binding fragment thereof that binds canine PD-1 comprises a canine antibody kappa light chain comprising a murine light chain CDR-1, CDR-2 and/or CDR-3 and a canine antibody heavy chain IgG comprising a murine heavy chain CDR-1, CDR-2 and/or CDR-3.

In other embodiments, the invention provides antibodies or antigen binding fragments thereof that specifically binds PD-1 and have canine antibody kappa light chains comprising one to six different CDRs comprising at least 80%, 85%, 90%, 95%, 98% or 99% sequence identity with the amino acid sequences of SEQ ID NOs: 13, 14, 15, 16, 17, 18, 19, 20, 21, 22, 23, 24, 25, and/or 26 and canine antibody heavy chain IgG comprising one to six different CDRs comprising at least 80%, 85%, 90%, 95%, 98% or 99% sequence identity with the amino acid sequences of SEQ ID NOs: 27, 28, 29, 30, 31, 32, 33, 34, 35, 36, 37, 38, and/or 114, while still exhibiting the desired binding and functional properties. In another embodiment the antibody or antigen binding fragment of the present invention comprises a canine frame comprising of a combination of IgG heavy chain sequence with a kappa light chain having one or more of the above-mentioned CDR amino acid sequences with 0, 1, 2, 3, 4, or 5 conservative or non-conservative amino acid substitutions, while still exhibiting the desired binding and functional properties.

Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

Sequence similarity includes identical residues and nonidentical, biochemically related amino acids. Biochemically related amino acids that share similar properties and may be interchangeable are discussed

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table 3 directly below.

TABLE 3
Exemplary Conservative Amino Acid Substitutions

| Original residue | Conservative substitution |
|---|---|
| Ala (A) | Gly; Ser; |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies of the invention are also contemplated by the present invention. “Function-conservative variants,” as used herein, refers to antibodies or fragments in which one or more amino acid residues have been changed without altering a desired property, such an antigen affinity and/or specificity. Such variants include, but are not limited to, replacement of an amino acid with one having similar properties, such as the conservative amino acid substitutions of Table 3 above.

Nucleic Acids

The present invention further comprises the nucleic acids encoding the immunoglobulin chains of murine anti-canine PD-1 and/or caninized murine anti-canine PD-1 antibodies and antigen binding fragments thereof disclosed herein (see Examples below).

Also included in the present invention are nucleic acids that encode immunoglobulin polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the CDRs and antibodies provided herein when the comparison is performed by a BLAST algorithm wherein the parameters of the algorithm are selected to give the largest match between the respective sequences over the entire length of the respective reference sequences. The present invention further provides nucleic acids that encode immunoglobulin polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm, wherein the parameters of the algorithm are selected to give the largest match between the respective sequences over the entire length of the respective reference sequences, are also included in the present invention.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141(1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70(1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

This present invention also provides expression vectors comprising the isolated nucleic acids of the invention, wherein the nucleic acid is operably linked to control sequences that are recognized by a host cell when the host cell is transfected with the vector. Also provided are host cells comprising an expression vector of the present invention and methods for producing the antibody or antigen binding fragment thereof disclosed herein comprising culturing a host cell harboring an expression vector encoding the antibody or antigen binding fragment in culture medium, and isolating the antigen or antigen binding fragment thereof from the host cell or culture medium.

Epitope Binding and Binding Affinity

The present invention further provides antibodies or antigen binding fragments thereof that bind to amino acid residues of the same epitope of canine PD-1 as the murine anti-canine PD-1 antibodies disclosed herein. In particular embodiments the murine anti-canine PD-1 antibodies or antigen binding fragments thereof are also capable of inhibiting/blocking the binding of canine PD-1 to canine PD-L1.

A caninized murine anti-canine PD-1 antibody can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern that the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo [See for example, Shinkawa et al., J. Biol. Chem. 278: 3466-3473 (2003); U.S. Pat. Nos. 6,946,292 and 7,214,775].

The present invention further includes antibody fragments of the murine anti-canine PD-1 antibodies disclosed herein. The antibody fragments include F(ab)₂ fragments, which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the Fc region between which disulfide bridges are located. An Fv fragment is a V_{L} or VH region.

In one embodiment, the antibody or antigen binding fragment comprises a heavy chain constant region, e.g., a canine constant region, such as IgG-A, IgG-B, IgG-C and IgG-D canine heavy chain constant region or a variant thereof. In another embodiment, the antibody or antigen binding fragment comprises a light chain constant region, e.g., a canine light chain constant region, such as lambda or kappa canine light chain region or variant thereof. By way of example, and not limitation, the canine heavy chain constant region can be from IgG-B and the canine light chain constant region can be from kappa.

Antibody Engineering

Caninized murine anti-canine PD-1 antibodies of the present invention can be engineered to include modifications to canine framework and/or canine frame residues within the variable domains of a parental (i.e., canine) monoclonal antibody, e.g. to improve the properties of the antibody.

Experimental and Diagnostic Uses

Murine anti-canine PD-1 and/or caninized murine anti-canine PD-1 antibodies or antigen-binding fragments thereof of the present invention may also be useful in diagnostic assays for canine PD-1 protein, e.g., detecting its expression in specific tumor cells, tissues, or serum. Such diagnostic methods may be useful in various disease diagnoses, particularly certain cancers in canines.

For example, such a method comprises the following steps:
(a) coat a substrate (e.g., surface of a microtiter plate well, e.g., a plastic plate) with a murine anti-canine PD-1 antibody or an antigen-binding fragment thereof;
(b) apply a sample to be tested for the presence of canine PD-1 to the substrate;
(c) wash the plate, so that unbound material in the sample is removed;
(d) apply detectably labeled antibodies (e.g., enzyme-linked antibodies) which are also specific to the PD-1 antigen;
(e) wash the substrate, so that the unbound, labeled antibodies are removed;
(f) if the labeled antibodies are enzyme linked, apply a chemical which is converted by the enzyme into a fluorescent signal; and
(g) detect the presence of the labeled antibody.

In a further embodiment, the labeled antibody is labeled with peroxidase which react with ABTS [e.g., 2,2′-azino-bis(3-ethylbenzthiazoline-6-sulphonic acid)] or 3,3′,5,5′-Tetramethylbenzidine to produce a color change which is detectable. Alternatively, the labeled antibody is labeled with a detectable radioisotope (e.g., ³H) which can be detected by scintillation counter in the presence of a scintillant. Murine anti-canine PD-1 antibodies of the invention may be used in a Western blot or immuno protein blot procedure.

Such a procedure forms part of the present invention and includes for example:
(i) contacting a membrane or other solid substrate to be tested for the presence of bound canine PD-1 or a fragment thereof with a murine anti-canine PD-1 antibody or antigen-binding fragment thereof of the present invention. Such a membrane may take the form of a nitrocellulose or vinyl-based [e.g., polyvinylidene fluoride (PVDF)] membrane to which the proteins to be tested for the presence of canine PD-1 in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contact of membrane with the murine anti-canine PD-1 antibody or antigen-binding fragment thereof, the membrane is optionally blocked, e.g., with non-fat dry milk or the like so as to bind non-specific protein binding sites on the membrane.
(ii) washing the membrane one or more times to remove unbound murine anti-canine PD-1 antibody or an antigen-binding fragment thereof and other unbound substances; and
(iii) detecting the bound murine anti-canine PD-1 antibody or antigen-binding fragment thereof.

Detection of the bound antibody or antigen-binding fragment may be by binding the antibody or antigen-binding fragment with a secondary antibody (an anti-immunoglobulin antibody) which is detectably labeled and, then, detecting the presence of the secondary antibody.

The murine anti-canine PD-1 antibodies and antigen-binding fragments thereof disclosed herein may also be used for immunohistochemistry. Such a method forms part of the present invention and comprises, e.g., (1) contacting a cell to be tested for the presence of canine PD-1 with a murine anti-canine PD-1 antibody or antigen-binding fragment thereof of the present invention; and (2) detecting the antibody or fragment on or in the cell. If the antibody or antigen-binding fragment itself is detectably labeled, it can be detected directly. Alternatively, the antibody or antigen-binding fragment may be bound by a detectably labeled secondary antibody which is detected.

Certain murine anti-canine PD-1 antibodies and antigen-binding fragments thereof disclosed herein may also be used for in vivo tumor imaging. Such a method may include injection of a radiolabeled murine anti-canine PD-1 antibodies or antigen-binding fragment thereof into the body of a canine to be tested for the presence of a tumor associated with canine PD-1 expression followed by nuclear imaging of the body of the patient to detect the presence of the labeled antibody or antigen-binding fragment e.g., at loci comprising a high concentration of the antibody or antigen-binding fragment which are bound to the tumor.

Imaging techniques include SPECT imaging (single photon emission computed tomography) or PET imaging (positron emission tomography). Labels include e.g., iodine-123 (¹²³I) and technetium-99m (^{99m}Tc), e.g., in conjunction with SPECT imaging or ¹¹C, N, ¹⁵O or ¹⁸F, e.g., in conjunction with PET imaging or Indium-111 [See e.g., Gordon et al., International Rev. Neurobiol. 67:385-440 (2005)].

Cross-Blocking Antibodies

Furthermore, an anti-canine PD-1 antibody or antigen-binding fragment thereof of the present invention includes any antibody or antigen-binding fragment thereof that binds to the same epitope in canine PD-1 to which the antibodies and fragments discussed herein bind and any antibody or antigen-binding fragment that cross-blocks (partially or fully) or is cross-blocked (partially or fully) by an antibody or fragment discussed herein for canine PD-1 binding; as well as any variant thereof.

The cross-blocking antibodies and antigen-binding fragments thereof discussed herein can be identified based on their ability to cross-compete with any of IBS, 3B6, 4D12, 7C9, 2H9, 5G5, and/or 2G9 in standard binding assays (e.g., BIACore®, ELISA, as exemplified below, or flow cytometry). For example, standard ELISA assays can be used in which a recombinant canine PD-1 protein is immobilized on the plate, one of the antibodies is fluorescently labeled and the ability of non-labeled antibodies to compete off the binding of the labeled antibody is evaluated. Additionally or alternatively, BIAcore® analysis can be used to assess the ability of the antibodies to cross-compete. The ability of a test antibody to inhibit the binding of, for example, IBS, 3B6, 4D12, 7C9, 2H9, 5G5, and/or 2G9, to canine PD-1 demonstrates that the test antibody can compete with IBS, 3B6, 4D12, 7C9, 2H9, 5G5, and/or 2G9 for binding to canine PD-1 and thus, may, in some cases, bind to the same epitope on canine PD-1 as IBS, 3B6, 4D12, 7C9, 2H9, 5G5, and/or 2G9. As stated above, antibodies and fragments that bind to the same epitope as any of the anti-canine PD-1 antibodies or fragments of the present invention also form part of the present invention.

Pharmaceutical Compositions and Administration

To prepare pharmaceutical or sterile compositions of a caninized murine anti-canine PD-1 antibody or antigen binding fragment thereof it can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, anti-PD-1 antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population).

The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the murine anti-canine PD-1 antibody or antigen binding fragment thereof can be administered by an invasive route such as by injection. In further embodiments of the invention, a murine anti-canine PD-1 antibody or antigen binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art. Alternately, one may administer a murine anti-canine or a caninized murine anti-canine PD-1 antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Antibodies or antigen binding fragments thereof disclosed herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 ag/kg, 100 g/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of a caninized murine anti-canine PD-1 antibody in the subject's serum, such as 0.1, 0.3, 1, 3, 10, 30, 100, 300 μg/ml or more. In other embodiments, a caninized murine anti-canine PD-1 antibody of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

The antigenic peptides recognized by anti-canine PD-1 and PDL-1 mAbs also may be used as vaccines to elicit antibodies that block the binding of PD-1 to PDL-1 and result in T cell activation and enhancement of the immune response. Such vaccines may be useful as therapeutic vaccines for diseases such as cancer or to act as enhancers of the immune response to other vaccines. In order to use these antigenic peptides as vaccines, one or more of these peptides may be coupled chemically or through the techniques of recombinant DNA technology to another carrier protein in order to enhance the immunogenicity of these peptides and elicit peptide-specific antibodies. Techniques for coupling peptides to carrier proteins are known to those skilled in the art. Peptide vaccines may be used to vaccinate animals by IM, S/C, oral, spray or in ovo routes. Peptide vaccines may be used as subunit proteins expressed from bacterial, viral, yeast or baculovirus virus systems. Alternatively such peptide vaccines may be delivered following administration of a variety of viral or bacterial vectors that express such peptide vaccines as can be practiced by methods known to those skilled in the art. The peptide vaccines may be administered in doses from 1-1000 μg and may optionally contain an adjuvant and an acceptable pharmaceutical carrier.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of a caninized murine anti-canine PD-1 antibody or antigen binding fragment thereof of the present invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

Other Combination Therapies

As previously described, a caninized murine anti-canine PD-1 antibody or antigen binding fragment thereof and/or an antigenic peptide of the present invention may be coadministered with one or other more therapeutic agents (such as a chemotherapeutic agent). The antibody may be linked to the agent (as an immunocomplex) or can be administered separately from the agent. In the latter case (separate administration), the antibody can be administered before, after or concurrently with the agent or can be co-administered with other known therapies.

Kits

Further provided are kits comprising one or more components that include, but are not limited to, an antibody or antigen binding fragment, as discussed herein, which specifically binds PD-1 (e.g., a caninized murine anti-canine PD-1 antibody or antigen binding fragment thereof) in association with one or more additional components including, but not limited to a pharmaceutically acceptable carrier and/or a chemotherapeutic agent, as discussed herein. The binding composition and/or the chemotherapeutic agent can be formulated as a pure composition or in combination with a pharmaceutically acceptable carrier, in a pharmaceutical composition.

In one embodiment, the kit includes a binding composition of the present invention (e.g., a caninized murine anti-canine PD-1 or a pharmaceutical composition thereof in one container (e.g., in a sterile glass or plastic vial) and a pharmaceutical composition thereof and/or a chemotherapeutic agent in another container (e.g., in a sterile glass or plastic vial).

If the kit includes a pharmaceutical composition for parenteral administration to a subject, the kit can also include a device for performing such administration. For example, the kit can include one or more hypodermic needles or other injection devices as discussed above. The kit can also include a package insert including information concerning the pharmaceutical compositions and dosage forms in the kit. Generally, such information aids pet owners and veterinarians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

As a matter of convenience, an antibody or specific binding agent disclosed herein can be provided in a kit, i.e., a packaged combination of reagents in predetermined amounts with instructions for performing the diagnostic or detection assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### EXAMPLES

### Example 1

### Canine PD-1 and PD-L1

Identification and Cloning of Canine PD-1:

A nucleic acid encoding a full length canine PD-1 (cPD-1) was identified through a search of the NCBI gene bank data bases (accession number XM_543338.4, SEQ ID NO: 1). The translated amino acid sequence SEQ ID NO: 2 (accession number XP-543338.3) corresponds to putative canine PD-1 protein which was further identified through searching the gene bank (NCBI) protein databases and aligning the identified amino acid sequence with murine, feline, and human PD-1 amino acid sequences. The DNA sequence corresponding to the full length canine PD-1 gene that was codon optimized for CHO cells was synthesized and cloned into a plasmid designated p96793. Comparison of DNA and protein sequences of predicted canine PD-1 with known PD-1 DNA and protein sequences led to the identification of the DNA sequences encoding the extra-cellular domain (ECD) of canine PD-1 (SEQ ID NO: 3) and the amino acid sequence of the ECD of canine PD-1 (SEQ ID NO: 4).

A DNA sequence encoding the ECD of canine PD-1 in addition to a GT linker and 8 histidine residues was synthesized and cloned into a plasmid designated LPD2726. A nucleic acid sequence (SEQ ID NO: 5) corresponding to the canine PD-1 ECD plus a GT linker and the Fc part of human IgG1 Fc gene was chemically synthesized and cloned into a plasmid designated LPD2727. Canine PD-1 ECD and the Fc part of human IgG1 Fc comprises the amino acid sequence of SEQ ID NO: 6.

Identification and Cloning of Canine PD-L1:

A nucleic acid encoding a full length canine PD-L1 was identified through a search of the NCBI gene bank data bases (accession number XM_541302.4; SEQ ID NO: 7). The translated amino acid sequence (accession number XP-541302.4; SEQ ID NO: 8) corresponding to the putative canine PD-L1 protein was identified by searching the gene bank (NCBI) protein databases and alignment of the identified sequence with known PD-L1 mouse and human sequences.

Comparison of DNA encoding canine PD-L1 with known PD-L1 sequences identified the DNA sequence corresponding to the ECD domain of canine PD-L1 (SEQ ID NO: 9; which was codon optimized for CHO cells). The predicted amino acid sequence of the ECD of canine PD-L1 is SEQ ID NO: 10. DNA encoding PD-L1 ECD plus GT linker and 8 histidine residues was synthesized and cloned into a plasmid designated LPD2695.

A DNA sequence encoding the amino acid sequence of canine PD-L1 ECD plus GT linker and the Fc part of human IgG1 Fc (SEQ ID NO: 11) was chemically synthesized and cloned into a plasmid designated LPD2697. Canine PD-L1 ECD plus GT linker and the Fc part of human IgG1 comprises the amino acid sequence of SEQ ID NO: 12. Table 4 contains a description of the expression plasmids mentioned above.

TABLE 4
PLASMIDS COMPRISING DNA ENCODING PD-1 or PD-L1

| PLASMID NAME | EXPRESSED GENE |
|---|---|
| P96793 | Canine PD-1 |
| LPD2726 | Canine PD-1 ECD-8HIS |
| LPD2727 | Canine PD-1 ECD−/Human IgG1 Fc |
| LPD2695 | Canine PD-L1 ECD-8HIS |
| LPD2697 | Canine PD-L1 ECD−/Human IgG1 Fc |

Expression of PD-1 and PD-L1 Proteins:

Expression plasmids encoding the PD-1ECD-HIS, PD-1ECD-Fc, PDL-1 ECD-HIS, and PD-L1ECD-Fc proteins were transfected into HEK 293 cells and the proteins were purified from the supernatant of transfected cells using Protein A for Fc fusion proteins or Nickel (Ni²⁺) column chromatography for HIS-tagged proteins. Purified proteins were used for: ELISA or binding assays as detailed below. Expressed proteins were analyzed by SDS-PAGE gels.

Full length canine PD-1 DNA sequence: signal
sequence underlined and in bold
SEQ ID NO: 1 is without the signal sequence;
SEQ ID NO: 105 is with the signal sequence.
atggggagccggcgggggccctggccgctcgtctgggccgtgctgcagct
gggctggtggccaggatggctcctagactcccctgacaggccctggagcc
cgctcaccttctccccggcgcagctcacggtgcaggagggagagaacgcc
acgttcacctgcagcctggccgacatccccgacagcttcgtgctcaactg
gtaccgcctgagcccccgcaaccagacggacaagctggccgccttccagg
aggaccgcatcgagccgggccgggacaggcgcttccgcgtcatgcggctg
cccaacgggcgggacttccacatgagcatcgtcgctgcgcgcctcaacga
cagcggcatctacctgtgcggggccatctacctgccccccaacacacaga
tcaacgagagtccccgcgcagagctctccgtgacggagagaaccctggag
ccccccacacagagccccagccccccacccagactcagcggccagttgca
ggggctggtcatcggcgtcacgagcgtgctggtgggtgtcctgctactgc
tgctgctgacctgggtcctggccgctgtcttccccagggccacccgaggt
gcctgtgtgtgcgggagcgaggacgagcctctgaaggagggccccgatgc
agcgcccgtcttcaccctggactacggggagctggacttccagtggcgag
agaagacgccggagcccccggcgccctgtgccccggagcagaccgagtat
gccaccatcgtcttcccgggcaggccggcgtccccgggccgcagggcctc
ggccagcagcctgcagggagcccagcctccgagccccgaggacggacccg
gcctgtggcccctctga
Full length canine PD-1 Amino acid sequence:
signal sequence underlined and in bold
SEQ ID NO: 2 is without the signal sequence;
SEQ ID NO: 106 is with the signal sequence.
MGSRRGPWPLVWAVLQLGWWPGWLLDSPDRPWSPLTFSPAQLTVQEGENA
TFTCSLADIPDSFVLNWYRLSPRNQTDKLAAFQEDRIEPGRDRRERVMRL
PNGRDFHMSIVAARLNDSGIYLCGAIYLPPNTQINESPRAELSVTERTLE
PPTQSPSPPPRLSGQLQGLVIGVTSVLVGVLLLLLLTWVLAAVFPRATRG
ACVCGSEDEPLKEGPDAAPVFTLDYGELDFQWREKTPEPPAPCAPEQTEY
ATIVFPGRPASPGRRASASSLQGAQPPSPEDGPGLWPL
Canine PD-1 extracellular domain_DNA sequence:
SEQ ID NO: 3 (Codon optimized for expression in
CHO cells)
ctggattcccccgacagaccctggagccctctcaccttctcccctgccca
gctgaccgtccaggaaggcgagaatgccaccttcacctgcagcctcgccg
acatccccgacagcttcgtgctgaactggtacagactgagccccaggaac
cagaccgacaagctggccgctttccaggaggacaggatcgaacccggcag
ggacaggaggtttagggtcatgaggctgcccaacggcagggacttccaca
tgtccatcgtggccgccagactgaacgactccggcatctacctgtgcggc
gctatctacctgccccccaacacccagatcaacgagagccccagggccga
actgagcgtgacagagagaaccctggaacctcccacccagagcccttccc
ctcctcctagactgagcggacagctgcagggcctggtg
Canine PD-1 extracellular domain: SEQ ID NO: 4:
LDSPDRPWSPLTFSPAQLTVQEGENATFTCSLADIPDSFVLNWYRLSPRN
QTDKLAAFQEDRIEPGRDRRFRVMRLPNGRDFHMSIVAARLNDSGIYLCG
AIYLPPNTQINESPRAELSVTERTLEPPTQSPSPPPRLSGQLQGLV
Canine PD-1 extracellular domain - human IgG1
Fc DNA sequence: SEQ ID NO: 5 (Codon optimized
for expression in HEK-293 cells)
ctggattcccccgacagaccctggagccctctcaccttctcccctgccca
gctgaccgtccaggaaggcgagaatgccaccttcacctgcagcctcgccg
acatccccgacagcttcgtgctgaactggtacagactgagccccaggaac
cagaccgacaagctggccgctttccaggaggacaggatcgaacccggcag
ggacaggaggtttagggtcatgaggctgcccaacggcagggacttccaca
tgtccatcgtggccgccagactgaacgactccggcatctacctgtgcggc
gctatctacctgccccccaacacccagatcaacgagagccccagggccga
actgagcgtgacagagagaaccctggaacctcccacccagagcccttccc
ctcctcctagactgagcggacagctgcagggcctggtgggtaccgacaaa
actcacacatgcccaccgtgcccagcacctgaactcctggggggaccgtc
agtcttcctcttccccccaaaacccaaggacaccctcatgatctcccgga
cccctgaggtcacatgcgtggtggtggacgtgagccacgaagaccctgag
gtcaagttcaactggtacgtggacggcgtggaggtgcataatgccaagac
aaagccgcgggaggagcagtacaacagcacgtaccgtgtggtcagcgtcc
tcaccgtcctgcaccaggactggctgaatggcaaggagtacaagtgcaag
gtctccaacaaagccctcccagcccccatcgagaaaaccatctccaaagc
caaagggcagccccgagaaccacaggtgtacaccctgcccccatcccggg
atgagctgaccaagaaccaggtcagcctgacctgcctggtcaaaggcttc
tatcccagcgacatcgccgtggagtgggagagcaatgggcagccggagaa
caactacaagaccacgcctcccgtgctggactccgacggctccttcttcc
tctacagcaagctcaccgtggacaagagcaggtggcagcaggggaacgtc
ttctcatgctccgtgatgcatgaggctctgcacaaccactacacgcagaa
gagcctctccctgtctccgggtaaatga
Canine PD-1 extracellular domain - human IgG1 Fc
fusion protein: signal sequence underlined and in
bold:
SEQ ID NO: 6 is without the signal sequence; SEQ
ID NO: 113 is with the signal sequence
MNFLLSWVHWSLALLLYLHHAKWSQALDSPDRPWSPLTFSPAQLTVQEGE
NATFTCSLADIPDSFVLNWYRLSPRNQTDKLAAFQEDRIEPGRDRRERVM
RLPNGRDFHMSIVAARLNDSGIYLCGAIYLPPNTQINESPRAELSVTERT
LEPPTQSPSPPPRLSGQLQGLVGTDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
Full length canine PD-L1 DNA sequence: signal
sequence underlined and in bold
SEQ ID NO: 7 is without the signal sequence;
SEQ ID NO: 107 is with the signal sequence.
atgagaatgtttagtgtctttacattcatggcctactgccatttgctaaa
agcatttacgatcacagtttctaaggacctgtatgtggtagagtatggtg
gcaatgtgacaatggaatgcaaattcccggtggaaaaacagttaaacttg
tttgcactaatcgtctactgggaaatggaggataaaaaaattatacaatt
tgtgaatggaaaggaagacctgaaagttcagcacagcagctacagccaga
gggctcagctattgaaggaccagctcttcttggggaaggctgcgcttcag
atcacagatgtgagattgcaggatgcaggggtttactgctgcttgatcgg
ctatggcggtgctgactacaagcggattactttgaaagttcatgccccgt
accgcaacatcagccaaagaatttctgtggatcctgtcacctctgaacat
gaactaatgtgtcaggctgagggttaccctgaggctgaagtcatctggac
aagcagtgaccaccgagtcctgagtggcaaaaccaccatcactaattcca
atagggaagagaagcttttcaatgtgaccagcacgctgaacatcaatgca
acagctaatgagattttctactgcacttttcaaagatcaggtcctgagga
aaacaatactgccgagttggtcatcccagaacgactgcccgttccagcaa
gtgagaggactcatttcatgattctgggacctttcctgttgcttcttggt
gtagtcctggcagtcactttctgtctaaaaaaacatgggagaatgatgga
tgtggaaaaatgttgcacccgagataggaactcaaagaaacgaaatgata
tacaatttgaagagacataa
Full length canine PD-L1: signal sequence
underlined and in bold
SEQ ID NO: 8 is without the signal sequence;
SEQ ID NO: 108 is with the signal sequence.
MRMESVFTFMAYCHLLKAFTITVSKDLYVVEYGGNVTMECKFPVEKQLNL
FALIVYWEMEDKKIIQFVNGKEDLKVQHSSYSQRAQLLKDQLFLGKAALQ
ITDVRLQDAGVYCCLIGYGGADYKRITLKVHAPYRNISQRISVDPVTSEH
ELMCQAEGYPEAEVIWTSSDHRVLSGKTTITNSNREEKLFNVTSTLNINA
TANEIFYCTFQRSGPEENNTAELVIPERLPVPASERTHFMILGPFLLLLG
VVLAVTFCLKKHGRMMDVEKCCTRDRNSKKRNDIQFEET
Canine PD-L1 extracellular domain DNA sequence:
SEQ ID NO: 9 (Codon optimized for expression in
CHO cells)
tttaccatcaccgtgtccaaggacctgtacgtggtcgagtacggcggcaa
tgtgaccatggagtgcaagttccccgtggagaagcagctgaacctgttcg
ccctcatcgtgtactgggagatggaggacaagaagatcatccagttcgtg
aacggcaaggaggacctgaaggtgcagcactccagctactcccagagagc
ccagctgctgaaggaccagctgttcctgggcaaggccgccctgcagatca
ccgacgtgagactgcaggacgccggcgtgtattgctgcctgatcggctac
ggaggcgccgactacaagaggatcaccctgaaggtgcatgcaccctacag
gaacatcagccagaggatcagcgtcgatcccgtgaccagcgagcacgagc
tgatgtgccaagccgagggctatcccgaggccgaagtgatctggaccagc
agcgaccacagggtcctgagcggcaagaccaccatcaccaacagcaacag
ggaggagaagctgttcaacgtgaccagcaccctcaacatcaacgccaccg
ccaacgagatcttctactgcaccttccagaggagcggccccgaagagaac
aacaccgccgagctggtgatccccgagagactgcctgtgcctgccagcga
gaggacccac
Canine PD-L1 extracellular domain protein:
SEQ ID NO: 10
FTITVSKDLYVVEYGGNVTMECKFPVEKQLNLFALIVYWEMEDKKIIQFV
NGKEDLKVQHSSYSQRAQLLKDQLFLGKAALQITDVRLQDAGVYCCLIGY
GGADYKRITLKVHAPYRNISQRISVDPVTSEHELMCQAEGYPEAEVIWTS
SDHRVLSGKTTITNSNREEKLFNVTSTLNINATANEIFYCTFQRSGPEEN
NTAELVIPERLPVPASERTH
Canine PD-L1 extracellular domain - human IgG1
Fc DNA sequence: SEQ ID NO: 11 (Codon optimized
for expression in HEK-293 cells)
tttaccatcaccgtgtccaaggacctgtacgtggtcgagtacggcggcaa
tgtgaccatggagtgcaagttccccgtggagaagcagctgaacctgttcg
ccctcatcgtgtactgggagatggaggacaagaagatcatccagttcgtg
aacggcaaggaggacctgaaggtgcagcactccagctactcccagagagc
ccagctgctgaaggaccagctgttcctgggcaaggccgccctgcagatca
ccgacgtgagactgcaggacgccggcgtgtattgctgcctgatcggctac
ggaggcgccgactacaagaggatcaccctgaaggtgcatgcaccctacag
gaacatcagccagaggatcagcgtcgatcccgtgaccagcgagcacgagc
tgatgtgccaagccgagggctatcccgaggccgaagtgatctggaccagc
agcgaccacagggtcctgagcggcaagaccaccatcaccaacagcaacag
ggaggagaagctgttcaacgtgaccagcaccctcaacatcaacgccaccg
ccaacgagatcttctactgcaccttccagaggagcggccccgaagagaac
aacaccgccgagctggtgatccccgagagactgcctgtgcctgccagcga
gaggacccacggtaccgacaaaactcacacatgcccaccgtgcccagcac
ctgaactcctggggggaccgtcagtcttcctcttccccccaaaacccaag
gacaccctcatgatctcccggacccctgaggtcacatgcgtggtggtgga
cgtgagccacgaagaccctgaggtcaagttcaactggtacgtggacggcg
tggaggtgcataatgccaagacaaagccgcgggaggagcagtacaacagc
acgtaccgtgtggtcagcgtcctcaccgtcctgcaccaggactggctgaa
tggcaaggagtacaagtgcaaggtctccaacaaagccctcccagccccca
tcgagaaaaccatctccaaagccaaagggcagccccgagaaccacaggtg
tacaccctgcccccatcccgggatgagctgaccaagaaccaggtcagcct
gacctgcctggtcaaaggcttctatcccagcgacatcgccgtggagtggg
agagcaatgggcagccggagaacaactacaagaccacgcctcccgtgctg
gactccgacggctccttcttcctctacagcaagctcaccgtggacaagag
caggtggcagcaggggaacgtcttctcatgctccgtgatgcatgaggctc
tgcacaaccactacacgcagaagagcctctccctgtctccgggtaaatga
Canine PD-L1 extracellular domain - human IgG1
Fc fusion protein: SEQ ID NO: 12
FTITVSKDLYVVEYGGNVTMECKFPVEKQLNLFALIVYWEMEDKKIIQFV
NGKEDLKVQHSSYSQRAQLLKDQLFLGKAALQITDVRLQDAGVYCCLIGY
GGADYKRITLKVHAPYRNISQRISVDPVTSEHELMCQAEGYPEAEVIWTS
SDHRVLSGKTTITNSNREEKLFNVTSTLNINATANEIFYCTFQRSGPEEN
NTAELVIPERLPVPASERTHGTDKTHTCPPCPAPELLGGPSVFLEPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

### Example 2

### Anti-Canine PD-1 Antibodies

Generation of anti-Canine PD1 monoclonal antibodies: A total of three Balb/c mice were immunized multiple times (with 10 μg each time) over a 17 day period. The immunizing antigen was the canine PD-1 ECD-Fc fusion protein. Following immunization, serum was collected from each mouse and tested for reactivity with canine PD-1 ECD-HIS tagged protein. The spleen cells of the mouse with the highest serum anti-PD-1 ECD-HIS titer were fused to the myeloma P3X63Ag8.653 cell line. Approximately 2 weeks following fusion, supernatant from putative hybridoma cells were tested by ELISA for their reactivity to the PD-1 ECD-HIS tagged protein. Hybridomas producing strong positive signals in the ELISA were subcloned by limiting dilution and tested again for reactivity to canine PD-1 ECD-HIS tagged protein.

Confirmation of Monoclonal Murine Antibodies Reactivity Against Canine PD-1:

The reactivity of antibodies secreted by hybridomas to ECD of canine PD-1 was confirmed by ELISA. Hybridoma cells were cultured using CELLine bioreactors (Integra-biosciences) for 10-30 days. Cells were initially maintained in DMEM supplemented with 4 mM L-glutamine and 10% Ultra Low IgG fetal bovine serum (FBS) from Gibco. Hybridoma cells were seeded in CELLine bioreactor cell chambers at a cell density of approximately 2×10⁶ cells/mL in 15 mL of the same medium with the FBS concentration increased to 20%. The outer chamber was filled with 1 L of nutrient medium (DMEM with 4 mM L-glutamine and 2% standard FBS). Hybridoma cells in the cell chamber were expanded to approximately 2.5×10⁷ cells/mL over 3-7 days. Then, 10 mL of cell suspension was harvested from the cell chamber and replaced with fresh media to allow for re-expansion of cells and subsequent harvests. This procedure was repeated as necessary to obtain adequate amounts of mAb from each hybridoma clone. Harvested cell suspensions were centrifuged and the supernatants were filtered through 0.2 micron filter membranes. For antibody purification, each clone's supernatant was purified using a Protein G Sepharose 4 Fast flow 5 mL column (GE Healthcare) by gravity flow. After washing with Tris-EDTA (TE) buffer pH 8.0, bound antibodies were eluted using 0.1 M glycine buffer, pH 2.7, followed by pH neutralization using 1 M Tris, pH 8.0. Antibodies were concentrated and buffer exchanged into phosphate-buffered saline (PBS) using Centriprep YM-10.10 kDa NMWL centrifugal filter units (Millipore). Antibody concentrations were quantified using spectrophotometry.

Purified anti-canine PD-1 mAbs were tested for reactivity with the HIS-tagged ECD domain of canine PD-1 by ELISA as follows: HIS-tagged canine PD-1 ECD protein is diluted to 10 μg/mL in coating buffer (Carbonate/Bicarbonate pH 9.0) and dispensed at 100 μl/well in 96-well flat bottomed ELISA plates (NUNC). The plates are incubated at 4° C. overnight. The plates are then washed three times with phosphate buffered saline containing 0.05% Tween-20 (PBST). Next, 200 μl of blocking buffer (5% skim milk in PBST) is added to each well and the plates are incubated at 37° C. for 60 minutes. The plates are then washed three times with PBST. Next, 100 μl of test mAbs diluted in blocking buffer is added to the first wells of the appropriate columns. Test mAbs are then diluted two-fold to the appropriate plate position. Following incubation of the plates at 37° C. for 60 minutes, the plates are washed three times with PBST. Next, 100 μl per well of a 1:2,000 dilution of a horseradish peroxidase conjugated goat anti-mouse IgG (KPL) is added to the plates, which are then incubated at 37° C. for 60 minutes. Then the plates are washed three times with PBST, and 100 μl/well of 3,3′,5,5′ tetramethyl benzidine, (TMB) substrate (from KPL) is added to the plates. The color reaction is allowed to develop for 5-20 minutes at 37° C. prior to measuring absorbance at 650 nm.

CHO Cells Expressing Canine PD-1 Protein:

The full length canine PD-1 gene was cloned into plasmid p96793. In this plasmid the expression of the PD-1 protein is driven by an hCMV promoter. CHO DXB11 cells (dhfr-) were maintained in MEM-alpha (Gibco) supplemented with 10% fetal bovine serum. Transfection of CHO cells with plasmid p96793 was carried out in 75 cm² flasks containing approximately 6×10⁶ cells by liposome-mediated gene delivery using Lipofectamine (Invitrogen). After 48 hours, cells were passaged into MEM-alpha medium without nucleosides, supplemented with 10% FBS and 400 μg/mL hygromycin B (selective medium). Limited-dilution cloning was performed on the pool of dhfr+, hygromycin resistant cells. Clones were assessed for expression of canine PD-1 by immunofluorescence assay. Briefly, cell monolayers were fixed in 96 well plates with 80% acetone. Fixed and dried cell monolayers were then incubated for 1 hour with a polyclonal goat anti-human PD-1 antibody (R&D Systems). Plates were washed with PBS, then incubated for 1 hour with a fluorescein-labeled rabbit anti-goat IgG antibody (KPL). Plates were washed with PBS. Clones exhibiting fluorescence were expanded and cell stocks were established.

Reactivity of Mouse mAbs Against Canine PD-1 Proteins Expressed on CHO Cells'

The reactivity of mouse anti-canine PD-1 mAbs with canine PD-1 on CHO cells was determined by a cell-based assay using CHO cells that express PD-1. Briefly, the CHO cells expressing canine PD-1 were cultured to 80-100% confluency in 50 μl media (DMEM/HAM's F12, 10% FBS). Next, 50 μl of media containing various concentrations of purified mAbs were added for 1 hour at 37° C. Following three washes with PBS-Tween, 100 μl of goat anti-mouse horse raddish peroxidase (HRP) diluted 1:1000 in culture media was added for one hour at 37° C. After three additional washes with PBS-Tween, bound mAbs were visualized with a perioxidase substrate (TMB). The absorbance increase due to perioxidase activity at 450 nm was measured in a microplate reader.

Binding Studies of Mouse Anti-Canine PD-1 mAbs and Caninized Mouse Anti-Canine PD-1 mAbs with Canine PD-1

Approximately 70 resonance units (RU) of the canine PD-1 antigen was immobilized directly by amine coupling. Affinity measurements were made via label-free surface plasmon resonance based technology (e.g., Biacore® T200) with an association time of 300 seconds, a dissociation time of 1200 seconds, and at concentrations of 50, 100, 200 (×2) 400, and 800 nanomolar (nM). A fitting model of 1:1 binding was used. The antigen (canine PD-1) was immobilized on the sensor chip through amine coupling and the four antibodies as indicated in Table 5 below, were used as analytes that flowed through the antigen surface. The results demonstrated that the binding affinities of the anti-canine PD-1 antibodies of the present invention for the canine PD-1 antigen were strong, having nanomolar and even subnanomolar dissociation constants (Kd). Moreover, the mouse anti-canine PD-1 monoclonal antibody and the corresponding caninized mouse anti-canine PD-1 monoclonal antibody from the same clone yielded strikingly similar Kd values (see Table 5 below).

TABLE 5
Binding Constant Deteminations

| | kₒₙ (k¹) | k_{off} (k⁻¹) | Kd | Chi² | Rmax |
|---|---|---|---|---|---|
| Antibody | M⁻¹s⁻¹ | s⁻¹ | M | (RU²) | (RU) |
| Murine 2H9 | 2.3 × 10⁴ | ≤5 × 10^{-6#} | ≤2.0 × 10^{-10#} | 0.19 | 25.6 |
| Caninized | 1.0 × 10⁴ | 5.9 × 10⁻⁶ | 5.9 × 10⁻¹⁰ | 0.10 | 27.7 |
| 2H9 | | | | | |
| Murine 3B6 | 1.8 × 10⁴ | 3.4 × 10⁻⁵ | 2.0 × 10⁻⁹ | 0.13 | 48.7 |
| Caninized | 1.6 × 10⁴ | 4.7 × 10⁻⁵ | 2.9 × 10⁻⁹ | 0.07 | 49.9 |
| 3B6 | | | | | |

^{#}The off-rate was so slow that it was below the detection limit of the instrument used.

Ligand Blockade by Mouse Anti-Canine PD1 mAbs:

A cell-based ELISA (CELISA) assay based on the CHO cell line expressing canine PD-1 was used for mouse mAbs which react with canine PD-1 (cPD-1). Ligand blockade was confirmed using this assay in conjunction with biotinylated cPD-L1/Fc protein. Briefly, seed cPD-1 CHO cells were placed in 96-well plates at 4×10⁴ cells per well and the cells were incubated at 37° C. for 18-24 hours till they are 95-100% confluent. The cell culture media was aspirated off, and the plates were washed 3× with PBS+0.05% Tween20 and 1×CHO media. Three-fold serial dilutions were made of anti-cPD1 mAbs in CHO media, starting at 30 μg/mL, and 50 μL/well of each antibody dilution were added down the plate. The incubation was performed at 37° C., 5% CO₂ with shaking for 30 min. 50 μL/well of cPD-L1-Fc-biotin (2 μg/ml in CHO media stock) was added and the incubation at 37° C., 5% CO₂ was continued with shaking for 45 min. The plates were washed 6 times with PBS+0.05% Tween 20. 100 ul/well of Streptavidin-HRP (1:2000) in CHO media was added followed by a incubation for 30-60 min at 37° C./5% CO₂. The plates were washed 5 times with PBS+0.05% Tween20 and then 100 μl/well of TMB color developing substrate was added. The color development was stopped by adding 50 μl/well of 1M phosphoric acid. The optical density (O.D.) at A450-A620 was measured using an ELISA plate reader.

Reactivity of Mouse mAbs with PD-1 Expressed on PBMC from Healthy and Cancer-Ridden Dogs:

PBMC were prepared from EDTA blood samples obtained from healthy dogs and dogs with cancer, using Ficoll separation. PBMC were resuspended in FACS buffer (PBS, 1% FBS, and 0.1% sodium azide) added at a concentration of 2.5×10⁵ cells per well, and incubated with test monocloncal antibodies (mAb) at various concentrations. Cells were incubated for 30 min at room temp, then washed twice. Cells were then resuspended and incubated with Alexa-488 conjugated donkey anti-mouse IgG (H+L chain) for 30 min at room temp, then washed twice. Cells were then incubated with PB and PE conjugated antibodies to canine CD4 and CD8 for 30 min, and then washed. Cells were then resuspended in FACS buffer and analyzed by flow cytometry to determine the percentage of CD4 or CD8 T cells positive for binding of the PD-1 mAbs. Controls included cells incubated with secondary antibody only, or with irrelevant isotype matched mAbs.

Cytokine Release from PBMC Obtained from Healthy and Cancer-Ridden Dogs:

PBMC were prepared from EDTA blood samples obtained from healthy dogs and dogs with cancer, using Ficoll separation. Cells were washed 3 times, and resuspended in complete tissue culture medium at a concentration of 2.5×10⁵ cells per well in triplicate wells in 96-well plates. Cells were activated with concanavalin A at 1 μg/ml. Test antibodies were added at various concentrations and the cultures were incubated for 96 hours. Controls included cells incubated with conA and no antibody, or conA and irrelevant isotype-matched antibodies. After 96 hours in culture, supernatants were collected and assayed for IFN-gamma release, using a commercial canine IFN-gamma ELISA kit (R & D Systems).

Cloning and Identification of DNA Sequences Corresponding to Mouse mAbs Variable Regions:

The DNA sequence of mouse VH and VL chains and the DNA sequences encoding their CDRs are identified following isolation of mRNA from each hybridoma using standard molecular biology methods. The SEQ ID NOs. of predicted amino acid sequences of the CDRs from these hybridomas are listed below:

Notably, there is substantial homology between the amino acid sequences of the CDRs for each of the seven mouse anti-Canine PD-1 antibodies exemplified.

CDR AMINO ACID SEQUENCES

SEQ
ID
NO.

VL CDR1

| | | |
|---|---|---|
| 1B5 | Lys Ser Ser Gln Ser Leu Leu Asn Ser Val Asn Gln Lys Asn Tyr Leu Ala | 13 |
| 2G9 | Arg Ser Ser Gln Asn Ile Val His Ser Asn Gly Asn Thr Tyr Leu Glu | 14 |
| 2H9 | His Ala Ser Gln Asn Ile Asn Val Trp Leu Ser | 15 |
| 3B6 | Lys Ser Ser Gln Ser Leu Leu Asn Ser Val Asn Gln Lys Asn Tyr Leu Ala | 13 |
| 4D12 | Lys Ser Ser Gln Ser Leu Leu Asn Ser Val Asn Gln Lys Asn Tyr Leu Ala | 13 |
| 5G5 | His Ala Ser Gln Asn Ile Asn Val Trp Leu Ser | 15 |
| 7C9 | Lys Ser Ser Gln Ser Leu Leu Asn Ser Val Asn Gln Lys Asn Tyr Leu Ala | 13 |

VL CDR2

| | | |
|---|---|---|
| 1B5 | Phe Ala Ser Thr Arg Val Ser | 16 |
| 2G9 | Lys Val Ser Asn Arg Phe Ser | 17 |
| 2H9 | Lys Ala Ser His Leu His Thr | 18 |
| 3B6 | Phe Ala Ser Ala Arg Val Ser | 19 |
| 4D12 | Phe Ala Ser Thr Arg Ile Ser | 20 |
| 5G5 | Lys Ala Ser Asn Leu His Thr | 21 |
| 7C9 | Phe Ala Ser Thr Arg Val Ser | 16 |

VL CDR3

| | | |
|---|---|---|
| 1B5 | Gln Gln Tyr Phe Ser Thr Pro Leu Thr | 22 |
| 2G9 | Phe Gln Gly Ser His Val Pro Tyr Thr | 23 |
| 2H9 | Gln Gln Gly Gln Ser Trp Pro Leu Thr | 24 |
| 3B6 | Gln Gln Tyr Phe Ser Thr Pro Leu Thr | 25 |
| 4D12 | Gln Gln Tyr Phe Ser Thr Pro Leu Thr | 25 |
| 5G5 | Gln Gln Gly Gln Ser Tyr Pro Leu Thr | 26 |
| 7C9 | Gln Gln Tyr Phe Ser Thr Pro Leu Thr | 22 |

VH CDR1

| | | |
|---|---|---|
| 1B5 | Gly Tyr Thr Phe Thr Thr Tyr Gly Met Ser | 27 |
| 2G9 | Gly Tyr Thr Phe Thr Arg Tyr Asn Met His | 28 |
| 2H9 | Gly Phe Asn Ile Lys Asn Thr Tyr Met His | 29 |
| 3B6 | Gly Tyr Thr Phe Thr Thr Tyr Gly Met Ser | 27 |
| 4D12 | Gly Tyr Thr Phe Thr Thr Tyr Gly Met Ser | 27 |
| 5G5 | Gly Phe Asn Ile Lys Asn Thr Tyr Met His | 29 |
| 7C9 | Gly Phe Ser Leu Thr Ser Tyr Gly Val His | 30 |

VH CDR2

| | | |
|---|---|---|
| 1B5 | Trp Ile Asn Ile Tyr Ser Gly Ile Pro Thr Tyr Ala Asp Asp Phe Lys Gly | 31 |
| 2G9 | Thr Ile Tyr Pro Gly Tyr Gly Asp Thr Ser Tyr Asn Gln Lys Phe Lys Gly | 32 |
| 2H9 | Arg Ile Ala Pro Ala Asn Val Asp Thr Lys Tyr Ala Pro Lys Phe Gln Gly | 33 |
| 3B6 | Trp Ile Asn Ile Tyr Ser Gly Ile Pro Thr Tyr Ala Asp Asp Phe Lys Gly | 31 |
| 4D12 | Trp Ile Asn Ile Tyr Ser Gly Met Pro Thr Tyr Ala Asp Asp Phe Lys Gly | 34 |
| 5G5 | Arg Ile Asp Pro Ala Asn Val Asn Thr Lys Tyr Ala Pro Lys Phe Gln Gly | 35 |
| 7C9 | Trp Ile Asn Ile Tyr Ser Gly Ile Pro Thr Tyr Ala Asp Asp Phe Lys Gly | 31 |

VH CDR3

| | | |
|---|---|---|
| 1B5 | Phe Asp Gly Pro Asp Tyr | 36 |
| 2G9 | Glu Phe Ala Asp Asp Tyr Pro Ile Pro Pro Phe Asp Tyr | 37 |
| 2H9 | Ile Tyr Tyr Asp Tyr Asp Gly Asp Ile Asp Val | 38 |
| 3B6 | Phe Asp Gly Pro Asp Tyr | 36 |
| 4D12 | Phe Asp Gly Pro Asp Tyr | 36 |
| 5G5 | Ile Phe Tyr Asp Tyr Asp Gly Asp Ile Asp Val | 114 |
| 7C9 | Phe Asp Gly Pro Asp Tyr | 36 |

Canonical structures (classes) for VH chain CDRs

| | |
|---|---|
| mAbs: 4D12, 3B6, | CDR: H1-1; CDR2: H2-1; CDR3: H3-6 |
| 7C9, and 1B5: | |
| mAb: 5G5: | CDR: H1-1; CDR2: H2-1; CDR3: H3-11 |
| mAb: 2H9 | CDR: H1-1; CDR2: H2-2A; CDR3: H3-11 |
| mAb: 2G9 | CDR: H1-1; CDR2: H2-2A; CDR3: H3-13 |

Canonical structures (classes) for VL chain CDRs

| | |
|---|---|
| mAbs: 4D12, 3B6, 7C9, 1B5: | CDRL: L1-3; CDR2: L2-1; CDR3: L3-1 |
| mAb: 5G5: | CDR: L1-2A; CDR2: L2-1; CDR3: L3-1 |
| mAb: 2H9 | CDR: L1-2A; CDR2: L2-1; CDR3: L3-1 |
| mAb: 2G9 | CDR: L1-4; CDR2: L2-1; CDR3: L3-1 |

### Example 3

### Mutant Canine Igg-B Antibodies Specific to Pd-1

There are four known IgG heavy chain subtypes of dog IgG and they are referred to as IgG-A, IgG-B, IgG-C, and IgG-D. The two known light chain subtypes are referred to as lambda and kappa. However, besides binding and activating of canine immune cells, a canine or caninized antibody against PD-1 optimally has two attributes:
1. lack of effector functions such as antibody-dependent cytotoxicity (ADCC) and complement-dependent cytotoxicity (CDC), and
2. be readily purified on a large scale using industry standard technologies such as that based on protein A chromatography.

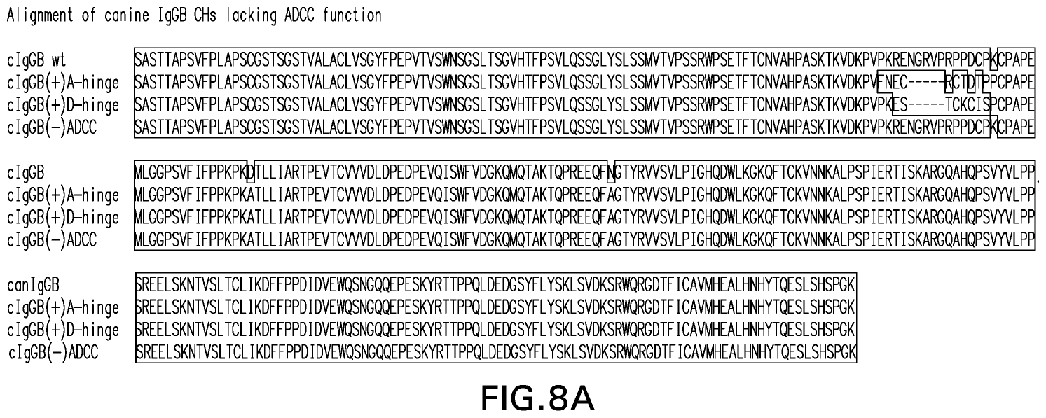

None of the naturally occurring canine IgG isotypes satisfy both criteria. For example, IgG-B can be purified using protein A, but has a high level of ADCC activity. IgG-C also has considerable ADCC activity. On the other hand, IgG-A binds weakly to protein A, but displays undesirable ADCC activity. Moreover, neither IgG-C nor IgG-D can be purified on protein A columns, although TgG-D display no ADCC activity. The present invention overcomes this difficulty by providing mutant canine IgG-B antibodies specific to PD-1; such antibodies lack effector functions such as ADCC and can be easily of purified using industry standard protein A chromatography. The exact modifications are shown in FIGS. 8A-8B.

The IgG-B variants with reduced effector functions described encompass a first IgG-B variant in which a lysine (D 277) and an asparagine (N 325) residue is each mutated to an alanine residue [cIgGB(−) ADCC], a second variant in which the hinge region of IgG-B is replaced by the hinge region of IgG-D [cIgGB(+) D-hinge], and a third variant in which the hinge region of IgG-B is replaced with the hinge region of IgG-A [cIgGB(+) A-hinge]. Additionally, the second and third variants also include replacement of the same lysine and asparagine residues of the first variant with an alanine residue. The numbering of the lysine and asparagine residues mutated in this invention is based on the numbering scheme described for canine IgG heavy chains in Tang et al., [Vet Immunol and Immunopathol, 80:259-270 (2001)].

Canine IgGB wt
SEQ ID NO: 39
SASTTAPSVFPLAPSCGSTSGSTVALACLVSGYFPEPVTVSWNSGSLTSG
VHTFPSVLQSSGLYSLSSMVTVPSSRWPSETFTCNVAHPASKTKVDKPVP
KRENGRVPRPPDCPKCPAPEMLGGPSVFIFPPKPKDTLLIARTPEVTCVV
VDLDPEDPEVQISWFVDGKQMQTAKTQPREEQFNGTYRVVSVLPIGHQDW
LKGKQFTCKVNNKALPSPIERTISKARGQAHQPSVYVLPPSREELSKNTV
SLTCLIKDFFPPDIDVEWQSNGQQEPESKYRTTPPQLDEDGSYFLYSKLS
VDKSRWQRGDTFICAVMHEALHNHYTQESLSHSPGK
Canine IgGB(+)A-hinge
SEQ ID NO: 40
SASTTAPSVFPLAPSCGSTSGSTVALACLVSGYFPEPVTVSWNSGSLTSG
VHTFPSVLQSSGLYSLSSMVTVPSSRWPSETFTCNVAHPASKTKVDKPVF
NECRCTDTPPCPAPEMLGGPSVFIFPPKPKATLLIARTPEVTCVVVDLDP
EDPEVQISWFVDGKQMQTAKTQPREEQFAGTYRVVSVLPIGHQDWLKGKQ
FTCKVNNKALPSPIERTISKARGQAHQPSVYVLPPSREELSKNTVSLTCL
IKDFFPPDIDVEWQSNGQQEPESKYRTTPPQLDEDGSYFLYSKLSVDKSR
WQRGDTFICAVMHEALHNHYTQESLSHSPGK
Canine IgGB(+)D-hinge
SEQ ID NO: 41
SASTTAPSVFPLAPSCGSTSGSTVALACLVSGYFPEPVTVSWNSGSLTSG
VHTFPSVLQSSGLYSLSSMVTVPSSRWPSETFTCNVAHPASKTKVDKPVP
KESTCKCISPCPAPEMLGGPSVFIFPPKPKATLLIARTPEVTCVVVDLDP
EDPEVQISWFVDGKQMQTAKTQPREEQFAGTYRVVSVLPIGHQDWLKGKQ
FTCKVNNKALPSPIERTISKARGQAHQPSVYVLPPSREELSKNTVSLTCL
IKDFFPPDIDVEWQSNGQQEPESKYRTTPPQLDEDGSYFLYSKLSVDKSR
WQRGDTFICAVMHEALHNHYTQESLSHSPGK
Canine IgGB(−)ADCC
SEQ ID NO: 42
SASTTAPSVFPLAPSCGSTSGSTVALACLVSGYFPEPVTVSWNSGSLTSG
VHTFPSVLQSSGLYSLSSMVTVPSSRWPSETFTCNVAHPASKTKVDKPVP
KRENGRVPRPPDCPKCPAPEMLGGPSVFIFPPKPKATLLIARTPEVTCVV
VDLDPEDPEVQISWFVDGKQMQTAKTQPREEQFAGTYRVVSVLPIGHQDW
LKGKQFTCKVNNKALPSPIERTISKARGQAHQPSVYVLPPSREELSKNTV
SLTCLIKDFFPPDIDVEWQSNGQQEPESKYRTTPPQLDEDGSYFLYSKLS
VDKSRWQRGDTFICAVMHEALHNHYTQESLSHSPGK

### Example 4

### Antibody Sequence Information (from Example 2 Above)

The Leader sequence is underlined; the CDR sequences are in bold; and the Framework sequence are neither underlined nor in bold.

mAb 1B5: Heavy chain variable region, DNA
FR1-CDR1-FR2-CDR2-FR3-CDR3-FR4
(SEQ ID NO: 43)
cagatccagttggtacagtctggacctgaactgaagaagcctggagagacagtcaagatctcc
tgcaaggcttctgggtataccttcacaacctatggaatgagctgggtgaaacaggctccagga
aagggtttaaagtggatgggctggattaatatctactctggaatcccaacatatgctgatgac
ttcaagggacggtttgccttctctttggaaacctctgccagcactgcctatttgcagatcgac
aacctcaaaaatgaggacacggctacatatttctgtgcaagatttgatggtcccgactactgg
ggccaaggcaccactctcaccgtctcccca
mAb 1B5: Heavy chain variable region, protein
FR1-CDR1-FR2-CDR2-FR3-CDR3-FR4
(SEQ ID NO: 44)
QIQLVQSGPELKKPGETVKISCKASGYTFTTYGMSWVKQAPGKGLKWMGWINIYSGIPTYADD
FKGRFAFSLETSASTAYLQIDNLKNEDTATYFCARFDGPDYWGQGTTLTVSP
Gln Ile Gln Leu Val Gln Ser Gly Pro Glu Leu Lys Lys Pro Gly Glu
Thr Val Lys Ile Ser Cys Lys Ala Ser Gly Tyr Thr Phe Thr Thr Tyr
Gly Met Ser Trp Val Lys Gln Ala Pro Gly Lys Gly Leu Lys Trp Met
Gly Trp Ile Asn Ile Tyr Ser Gly Ile Pro Thr Tyr Ala Asp Asp Phe
Lys Gly Arg Phe Ala Phe Ser Leu Glu Thr Ser Ala Ser Thr Ala
Tyr Leu Gln Ile Asp Asn Leu Lys Asn Glu Asp Thr Ala Thr Tyr Phe
Cys Ala Arg Phe Asp Gly Pro Asp Tyr Trp Gly Gln Gly Thr Thr Leu
Thr Val Ser Pro
mAb 1B5: Light chain variable region, DNA
FR1-CDR1-FR2-CDR2-FR3-CDR3-FR4
(SEQ ID NO: 45)
gacattgtgatgacacagtctccatcctccctggctatgtcagtaggacagaaggtcactatg
agctgcaagtccagtcagagccttttaaatagtgtcaatcaaaagaactatttggcctggtac
cagcagaaaccaggacagtctcctaaagttctggtatactttgcatccactagggtatctggg
gtccctgatcgcttcataggcagtggatctgggacagatttcactcttaccatcaccagtgtg
caggctgaagacctgacaacttacctctgtcagcaatattttagcactcctctcacgttcggt
gctgggaccaagctggaaataaaa
mAb 1B5: Light chain variable region, protein
FR1-CDR1-FR2-CDR2-FR3-CDR3-FR4
(SEQ ID NO: 46)
DIVMTQSPSSLAMSVGQKVTMSCKSSQSLLNSVNQKNYLAWYQQKPGQSPKVLVYFASTRVSG
VPDRFIGSGSGTDFTLTITSVQAEDLTTYLCQQYFSTPLTFGAGTKLEIK
Asp Ile Val Met Thr Gln Ser Pro Ser Ser Leu Ala Met Ser Val Gly
Gln Lys Val Thr Met Ser Cys Lys Ser Ser Gln Ser Leu Leu Asn Ser
Val Asn Gln Lys Asn Tyr Leu Ala Trp Tyr Gln Gln Lys Pro Gly Gln
Ser Pro Lys Val Leu Val Tyr Phe Ala Ser Thr Arg Val Ser Gly Val
Pro Asp Arg Phe Ile Gly Ser Gly Ser Gly Thr Asp Phe Thr Leu Thr
Ile Thr Ser Val Gln Ala Glu Asp Leu Thr Thr Tyr Leu Cys Gln Gln
Tyr Phe Ser Thr Pro Leu Thr Phe Gly Ala Gly Thr Lys Leu Glu Ile
Lys
mAb 2G9: Heavy chain variable region, DNA
Leader sequence-FR1-CDR1-FR2-CDR2-FR3-CDR3-FR4
(SEQ ID NO: 47)
atgggattcagcaggatctttctcttcctcctgtcagtaactacaggtgtccactcccaggct
tatctacagcagtctggggctgagctggtgaggcctggggcctcagtgaagatctcctgcaag
gcttctggctacacatttaccagatacaatatgcactgggtaaagcagacacctagacagggc
ctggaatggattggaactatttatcccggatatggtgatacttcttacaatcagaaattcaag
ggcaaggccacactgactgtagacatatcctccagcacagcctacatgcagctcaccagcctg
acatctgaggactctgcggtctatttctgttcaagggagtttgccgatgattaccccattccc
ccctttgactactggggccaaggcaccactctcacagtctcctca
mAb 2G9: Heavy chain variable region, protein
Leader sequence-FR1-CDR1-FR2-CDR2-FR3-CDR3-FR4
(SEQ ID NO: 48)
MGFSRIFLFLLSVTTGVHSQAYLQQSGAELVRPGASVKISCKASGYTFTRYNMHWVKQTPRQG
LEWIGTIYPGYGDTSYNQKFKGKATLTVDISSSTAYMQLTSLTSEDSAVYFCSREFADDYPIP
PFDYWGQGTTLTVSS
Met Gly Phe Ser Arg Ile Phe Leu Phe Leu Leu Ser Val Thr Thr Gly
Val His Ser Gln Ala Tyr Leu Gln Gln Ser Gly Ala Glu Leu Val Arg
Pro Gly Ala Ser Val Lys Ile Ser Cys Lys Ala Ser Gly Tyr Thr Phe
Thr Arg Tyr Asn Met His Trp Val Lys Gln Thr Pro Arg Gln Gly Leu
Glu Trp Ile Gly Thr Ile Tyr Pro Gly Tyr Gly Asp Thr Ser Tyr Asn
Gln Lys Phe Lys Gly Lys Ala Thr Leu Thr Val Asp Ile Ser Ser Ser
Thr Ala Tyr Met Gln Leu Thr Ser Leu Thr Ser Glu Asp Ser Ala Val
Tyr Phe Cys Ser Arg Glu Phe Ala Asp Asp Tyr Pro Ile Pro Pro Phe
Asp Tyr Trp Gly Gln Gly Thr Thr Leu Thr Val Ser Ser
mAb 2G9: Light chain variable region, DNA
Leader sequence-FR1-CDR1-FR2-CDR2-FR3-CDR3-FR4
(SEQ ID NO: 49)
atgaagttgcctgttaggctgttggtgctgatgttctggattcctgcttccagcagtgatgtt
ttgatgacccaaactccactctccctgcctgtcagtcttggagatcaagcctccatctcttgt
agatctagtcagaacattgtacatagtaatggaaacacctacttagaatggtacctgcagaaa
ccaggccagtctccaaagctcctgatctacaaagtttccaaccgattttctggggtcccagac
aggttcagtggcagtggatcagggacagatttcacactcaagatcagcagagtggaggctgag
gatctgggaatttattactgctttcaaggttcacatgttccgtacacgttcggaggggggacc
aagctggaaataaaa
mAb 2G9: Light chain variable region, protein
Leader sequence-FR1-CDR1-FR2-CDR2-FR3-CDR3-FR4
(SEQ ID NO: 50)
MKLPVRLLVLMFWIPASSSDVLMTQTPLSLPVSLGDQASISCRSSQNIVHSNGNTYLEWYLQK
PGQSPKLLIYKVSNRFSGVPDRFSGSGSGTDFTLKISRVEAEDLGIYYCFQGSHVPYTFGGGT
KLEIK
Met Lys Leu Pro Val Arg Leu Leu Val Leu Met Phe Trp Ile Pro Ala
Ser Ser Ser Asp Val Leu Met Thr Gln Thr Pro Leu Ser Leu Pro Val
Ser Leu Gly Asp Gln Ala Ser Ile Ser Cys Arg Ser Ser Gln Asn Ile
Val His Ser Asn Gly Asn Thr Tyr Leu Glu Trp Tyr Leu Gln Lys Pro
Gly Gln Ser Pro Lys Leu Leu Ile Tyr Lys Val Ser Asn Arg Phe Ser
Gly Val Pro Asp Arg Phe Ser Gly Ser Gly Ser Gly Thr Asp Phe Thr
Leu Lys Ile Ser Arg Val Glu Ala Glu Asp Leu Gly Ile Tyr Tyr Cys
Phe Gln Gly Ser His Val Pro Tyr Thr Phe Gly Gly Gly Thr Lys Leu
Glu Ile Lys
mAb 2H9: Heavy chain variable region, DNA
Leader sequence-FR1-CDR1-FR2-CDR2-FR3-CDR3-FR4
(SEQ ID NO: 51)
atgaaattcagctgggtcatcttcttcctgatggcagtggttacaggggtcaattcagaggtt
cagctgcagcagtctgtggcagagcttgtgaggccaggggcctcagtcaagttgtcctgcaca
gcttctggcttcaacattaaaaacacctatatgcactggataaaacagaggcctgaacagggc
ctggagtggattggaaggattgctcctgcgaatgttgatactaaatatgccccgaagttccag
ggcaaggccactataactgcagacacatcctccaacacagcctacatgcagctcagcaccctg
acatcggaggacactgccatctattactgtgtcctgatctactatgattacgacggggacatc
gatgtctggggcacagggaccacggtcaccgtctcctca
mAb 2H9: Heavy chain variable region, protein
Leader sequence-FR1-CDR1-FR2-CDR2-FR3-CDR3-FR4
(SEQ ID NO: 52)
MKFSWVIFFLMAVVTGVNSEVQLQQSVAELVRPGASVKLSCTASGFNIKNTYMHWIKQRPEQG
LEWIGRIAPANVDTKYAPKFQGKATITADTSSNTAYMQLSTLTSEDTAIYYCVLIYYDYDGDI
DVWGTGTTVTVSS
Met Lys Phe Ser Trp Val Ile Phe Phe Leu Met Ala Val Val Thr Gly
Val Asn Ser Glu Val Gln Leu Gln Gln Ser Val Ala Glu Leu Val Arg
Pro Gly Ala Ser Val Lys Leu Ser Cys Thr Ala Ser Gly Phe Asn Ile
Lys Asn Thr Tyr Met His Trp Ile Lys Gln Arg Pro Glu Gln Gly Leu
Glu Trp Ile Gly Arg Ile Ala Pro Ala Asn Val Asp Thr Lys Tyr Ala
Pro Lys Phe Gln Gly Lys Ala Thr Ile Thr Ala Asp Thr Ser Ser Asn
Thr Ala Tyr Met Gln Leu Ser Thr Leu Thr Ser Glu Asp Thr Ala Ile
Tyr Tyr Cys Val Leu Ile Tyr Tyr Asp Tyr Asp Gly Asp Ile Asp Val
Trp Gly Thr Gly Thr Thr Val Thr Val Ser Ser
mAb 2H9: Light chain variable region, DNA
Leader sequence-FR1-CDR1-FR2-CDR2-FR3-CDR3-FR4
(SEQ ID NO: 53)
atgagggtccttgctgagctcctggggctgctgctgttctgctttttaggtgtgagatgtgac
atccagatgaaccagtctccatccagtctgtctgcatcccttggagacacaattaccatcact
tgccatgccagtcagaacattaatgtttggttaagttggtaccagcagagaccaggaaatatt
cctaaactattgatctataaggcttctcacttacacacaggcgtcccatcaaggtttagtggc
agtggatctggaacaggtttcacattaaccatcagcagcctgcagcctgaagacattgccact
tactactgtcaacagggtcaaagttggccgctcacgttcggtgctgggaccaaactggagctg
aaa
mAb 2H9: Light chain variable region, protein
Leader sequence-FR1-CDR1-FR2-CDR2-FR3-CDR3-FR4
(SEQ ID NO: 54)
MRVLAELLGLLLFCFLGVRCDIQMNQSPSSLSASLGDTITITCHASQNINVWLSWYQQRPGNI
PKLLIYKASHLHTGVPSRFSGSGSGTGFTLTISSLQPEDIATYYCQQGQSWPLTFGAGTKLEL
K
Met Arg Val Leu Ala Glu Leu Leu Gly Leu Leu Leu Phe Cys Phe Leu
Gly Val Arg Cys Asp Ile Gln Met Asn Gln Ser Pro Ser Ser Leu Ser
Ala Ser Leu Gly Asp Thr Ile Thr Ile Thr Cys His Ala Ser Gln Asn
Ile Asn Val Trp Leu Ser Trp Tyr Gln Gln Arg Pro Gly Asn Ile Pro
Lys Leu Leu Ile Tyr Lys Ala Ser His Leu His Thr Gly Val Pro Ser
Arg Phe Ser Gly Ser Gly Ser Gly Thr Gly Phe Thr Leu Thr Ile Ser
Ser Leu Gln Pro Glu Asp Ile Ala Thr Tyr Tyr Cys Gln Gln Gly Gln
Ser Trp Pro Leu Thr Phe Gly Ala Gly Thr Lys Leu Glu Leu Lys
mAb 3B6: Heavy chain variable region, DNA
Leader sequence-FR1-CDR1-FR2-CDR2-FR3-CDR3-FR4
(SEQ ID NO: 55)
atgggttggctgtggaacttgctattcctgatggcagctgcccaaagtgcccaaacacagatc
cagttggtacagtctggacctgaactgaagaagcctggagagacagtcaagatctcctgcaag
gcttctgggtataccttcacaacctatggaatgagctgggtgaaacaggctccaggaaagggt
ttaaagtggatgggctggattaatatctactctggaatcccaacatatgctgatgacttcaag
ggacgatttgccttctctttggaaacctctgccagcactgcctatttgcagatcgacaacctc
aaaaatgaggacacggctacatatttctgtgcaagatttgatggtcccgactactggggccaa
ggcaccactctcacagtctcctca
mAb 3B6: Heavy chain variable region, protein
(SEQ ID NO: 56)
MGWLWNLLFLMAAAQSAQTQIQLVQSGPELKKPGETVKISCKASGYTFTTYGMSWVKQAPGKG
LKWMGWINIYSGIPTYADDFKGRFAFSLETSASTAYLQIDNLKNEDTATYFCARFDGPDYWGQ
GTTLTVSS
Met Gly Trp Leu Trp Asn Leu Leu Phe Leu Met Ala Ala Ala Gln Ser
Ala Gln Thr Gln Ile Gln Leu Val Gln Ser Gly Pro Glu Leu Lys Lys
Pro Gly Glu Thr Val Lys Ile Ser Cys Lys Ala Ser Gly Tyr Thr Phe
Thr Thr Tyr Gly Met Ser Trp Val Lys Gln Ala Pro Gly Lys Gly Leu
Lys Trp Met Gly Trp Ile Asn Ile Tyr Ser Gly Ile Pro Thr Tyr Ala
Asp Asp Phe Lys Gly Arg Phe Ala Phe Ser Leu Glu Thr Ser Ala Ser
Thr Ala Tyr Leu Gln Ile Asp Asn Leu Lys Asn Glu Asp Thr Ala Thr
Tyr Phe Cys Ala Arg Phe Asp Gly Pro Asp Tyr Trp Gly Gln Gly Thr
Thr Leu Thr Val Ser Ser
mAb 3B6: Light chain variable region, DNA
Leader sequence-FR1-CDR1-FR2-CDR2-FR3-CDR3-FR4
(SEQ ID NO: 57)
atggaatcacagacccaggtcctcatgtttcttctgctctgggtatctggtgcctgtgcagac
attgtgatgacacagtctccatcctccctggctgtgtcagtaggacggaaggtcactatgagc
tgcaagtccagtcagagccttttaaatagtgtcaatcaaaagaactatttggcctggtaccag
cagaaaccaggacagtctcctaaagttctggtatactttgcatccgctagggtatctggggtc
cctgatcgcttcataggcagtggatctgggacagatttcactcttgccatcagcagtgtgcag
gctgaagacctgacaacttacttctgtcagcaatattttagcactcctctcacgttcggtgct
gggaccaagctggaactgaaa
mAb 3B6: Light chain variable region, protein
Leader sequence-FR1-CDR1-FR2-CDR2-FR3-CDR3-FR4
(SEQ ID NO: 58)
MESQTQVLMFLLLWVSGACADIVMTQSPSSLAVSVGRKVTMSCKSSQSLLNSVNQKNYLAWYQ
QKPGQSPKVLVYFASARVSGVPDRFIGSGSGTDFTLAISSVQAEDLTTYFCQQYFSTPLTFGA
GTKLELK
Met Glu Ser Gln Thr Gln Val Leu Met Phe Leu Leu Leu Trp Val Ser
Gly Ala Cys Ala Asp Ile Val Met Thr Gln Ser Pro Ser Ser Leu Ala
Val Ser Val Gly Arg Lys Val Thr Met Ser Cys Lys Ser Ser Gln Ser
Leu Leu Asn Ser Val Asn Gln Lys Asn Tyr Leu Ala Trp Tyr Gln Gln
Lys Pro Gly Gln Ser Pro Lys Val Leu Val Tyr Phe Ala Ser Ala Arg
Val Ser Gly Val Pro Asp Arg Phe Ile Gly Ser Gly Ser Gly Thr Asp
Phe Thr Leu Ala Ile Ser Ser Val Gln Ala Glu Asp Leu Thr Thr Tyr
Phe Cys Gln Gln Tyr Phe Ser Thr Pro Leu Thr Phe Gly Ala Gly Thr
Lys Leu Glu Leu Lys
mAb 4D12: Heavy chain variable region, DNA
Leader sequence-FR1-CDR1-FR2-CDR2-FR3-CDR3-FR4
(SEQ ID NO: 59)
atgggttggctgtggaacttgctattcctgatggcagctgcccaaagtgcccaagcacagatc
cagttggtacagtctggacctgaactgaagaagcctggagagacagtcaagatctcctgcaag
gcttctgggtataccttcacaacctatggaatgagctgggtgaaacaggcgccaggaaagggt
ttaaagtggatgggctggataaatatctactctggaatgccaacatatgctgatgacttcaag
ggacggtttgccttctctttggaaacctctgtcagcactgcctatttgcagatcaacaacctc
aaaaatgaggacacggctacatatttctgtgcaagatttgatggtcccgactactggggccaa
ggcaccactctcacagtctcctca
mAb 4D12: Heavy chain variable region, protein
Leader sequence-FR1-CDR1-FR2-CDR2-FR3-CDR3-FR4
(SEQ ID NO: 60)
MGWLWNLLFLMAAAQSAQAQIQLVQSGPELKKPGETVKISCKASGYTFTTYGMSWVKQAPGKG
LKWMGWINIYSGMPTYADDFKGRFAFSLETSVSTAYLQINNLKNEDTATYFCARFDGPDYWGQ
GTTLTVSS
Met Gly Trp Leu Trp Asn Leu Leu Phe Leu Met Ala Ala Ala Gln Ser
Ala Gln Ala Gln Ile Gln Leu Val Gln Ser Gly Pro Glu Leu Lys Lys
Pro Gly Glu Thr Val Lys Ile Ser Cys Lys Ala Ser Gly Tyr Thr
Phe Thr Thr Tyr Gly Met Ser Trp Val Lys Gln Ala Pro Gly Lys
Gly Leu Lys Trp Met Gly Trp Ile Asn Ile Tyr Ser Gly Met Pro Thr
Tyr Ala Asp Asp Phe Lys Gly Arg Phe Ala Phe Ser Leu Glu Thr
Ser Val Ser Thr Ala Tyr Leu Gln Ile Asn Asn Leu Lys Asn Glu Asp
Thr Ala Thr Tyr Phe Cys Ala Arg Phe Asp Gly Pro Asp Tyr Trp Gly
Gln Gly Thr Thr Leu Thr Val Ser Ser
mAb 4D12: Light chain variable region, DNA
Leader sequence-FR1-CDR1-FR2-CDR2-FR3-CDR3-FR4
(SEQ ID NO: 61)
atggaatcacagacccaggtcctcatgtttcttctgctctgggtatctggtgcctgtgcagac
attgtgatgacacagtctccatcctccctggctatgtcagtaggacagaaggtcactatgagc
tgcaagtccagtcagagccttttaaatagtgtcaatcaaaagaactatttggcctggtaccag
cagaaaccaggacagtctcctaaagttctggtatactttgcatccactaggatatctggggtc
cctgatcgcttcataggcagtggatctgggacagatttcactcttaccatcagcagtgtgcag
gctgaagacctggcagattacttctgtcagcaatattttagcactcctctcacgttcggtgct
gggaccaagctggagctgaaa
mAb 4D12: Light chain variable region, protein
Leader sequence-FR1-CDR1-FR2-CDR2-FR3-CDR3-FR4
(SEQ ID NO: 62)
MESQTQVLMFLLLWVSGACADIVMTQSPSSLAMSVGQKVTMSCKSSQSLLNSVNQKNYLAWYQ
QKPGQSPKVLVYFASTRISGVPDRFIGSGSGTDFTLTISSVQAEDLADYFCQQYFSTPLTFGA
GTKLELK
Met Glu Ser Gln Thr Gln Val Leu Met Phe Leu Leu Leu Trp Val Ser
Gly Ala Cys Ala Asp Ile Val Met Thr Gln Ser Pro Ser Ser Leu Ala
Met Ser Val Gly Gln Lys Val Thr Met Ser Cys Lys Ser Ser Gln Ser
Leu Leu Asn Ser Val Asn Gln Lys Asn Tyr Leu Ala Trp Tyr Gln Gln
Lys Pro Gly Gln Ser Pro Lys Val Leu Val Tyr Phe Ala Ser Thr Arg
Ile Ser Gly Val Pro Asp Arg Phe Ile Gly Ser Gly Ser Gly Thr Asp
Phe Thr Leu Thr Ile Ser Ser Val Gln Ala Glu Asp Leu Ala Asp Tyr
Phe Cys Gln Gln Tyr Phe Ser Thr Pro Leu Thr Phe Gly Ala Gly Thr
Lys Leu Glu Leu Lys
mAb 5G5: Heavy chain variable region, DNA
Leader sequence-FR1-CDR1-FR2-CDR2-FR3-CDR3-FR4
(SEQ ID NO: 63)
atgaaattcagctgggtcatcttcttcctgatggcagtggttacaggggtcaattcagaggtt
cagctgcagcagtctgtggcagagcttgtgaggccaggggcctcagtcaagttgtcctgcaca
gtttctggcttcaacattaaaaacacctatatgcactgggtgaagcagaggcctgaacagggc
ctggagtggattggaagaattgatcctgcgaatgttaatactaaatatgccccgaagttccag
ggcaaggccactataactacagacacatcctccaacacagcctacatgcagctcagcagcctg
acatcggaggacactgccatctattactgtgtcctgattttctatgattacgacggggacatc
gatgtctggggcacagggaccaaggtcaccgtctcctca
mAb 5G5: Heavy chain variable region, protein
Leader sequence-FR1-CDR1-FR2-CDR2-FR3-CDR3-FR4
(SEQ ID NO: 64)
MKFSWVIFFLMAVVTGVNSEVQLQQSVAELVRPGASVKLSCTVSGFNIKNTYMHWVKQRPEQG
LEWIGRIDPANVNTKYAPKFQGKATITTDTSSNTAYMQLSSLTSEDTAIYYCVLIFYDYDGDI
DVWGTGTKVTVSS
Met Lys Phe Ser Trp Val Ile Phe Phe Leu Met Ala Val Val Thr Gly
Val Asn Ser Glu Val Gln Leu Gln Gln Ser Val Ala Glu Leu Val Arg
Pro Gly Ala Ser Val Lys Leu Ser Cys Thr Val Ser Gly Phe Asn Ile
Lys Asn Thr Tyr Met His Trp Val Lys Gln Arg Pro Glu Gln Gly Leu
Glu Trp Ile Gly Arg Ile Asp Pro Ala Asn Val Asn Thr Lys Tyr Ala
Pro Lys Phe Gln Gly Lys Ala Thr Ile Thr Thr Asp Thr Ser Ser Asn
Thr Ala Tyr Met Gln Leu Ser Ser Leu Thr Ser Glu Asp Thr Ala Ile
Tyr Tyr Cys Val Leu Ile Phe Tyr Asp Tyr Asp Gly Asp Ile Asp Val
Trp Gly Thr Gly Thr Lys Val Thr Val Ser Ser
mAb 5G5: Light chain variable region, DNA
Leader sequence-FR1-CDR1-FR2-CDR2-FR3-CDR3-FR4
(SEQ ID NO: 65)
atgagggtccttgctgagctcctggggctgctgctgttctgctttttaggtgtgagatgtgac
atccagatgaaccagtctccatccagtctgtctgcatcccttggagacacaattaccatcact
tgccatgccagtcagaacattaatgtttggttaagctggtaccagcagaaaccaggaaatatt
cctaaactattgatctataaggcttccaacttacacacaggcgtcccatcaaggtttagtggc
agtggatctggaacagatttcacattaaccatcagcagcctgcagcctgaagacattgccact
tactactgtcaacagggtcaaagttatccgctcacgttcggtgctgggaccaagctggagctg
aaa
mAb 5G5: Light chain variable region, protein
Leader sequence-FR1-CDR1-FR2-CDR2-FR3-CDR3-FR4
(SEQ ID NO: 66)
MRVLAELLGLLLFCFLGVRCDIQMNQSPSSLSASLGDTITITCHASQNINVWLSWYQQKPGNI
PKLLIYKASNLHTGVPSRFSGSGSGTDFTLTISSLQPEDIATYYCQQGQSYPLTFGAGTKLEL
K
Met Arg Val Leu Ala Glu Leu Leu Gly Leu Leu Leu Phe Cys Phe Leu
Gly Val Arg Cys Asp Ile Gln Met Asn Gln Ser Pro Ser Ser Leu Ser
Ala Ser Leu Gly Asp Thr Ile Thr Ile Thr Cys His Ala Ser Gln Asn
Ile Asn Val Trp Leu Ser Trp Tyr Gln Gln Lys Pro Gly Asn Ile Pro
Lys Leu Leu Ile Tyr Lys Ala Ser Asn Leu His Thr Gly Val Pro Ser
Arg Phe Ser Gly Ser Gly Ser Gly Thr Asp Phe Thr Leu Thr Ile Ser
Ser Leu Gln Pro Glu Asp Ile Ala Thr Tyr Tyr Cys Gln Gln Gly Gln
Ser Tyr Pro Leu Thr Phe Gly Ala Gly Thr Lys Leu Glu Leu Lys
mAb 7C9: Heavy chain variable region, DNA
FR1-CDR1-FR2-CDR2-FR3-CDR3-FR4
(SEQ ID NO: 67)
caggtgcagctgaagcagtcaggacctggcctagtgcagccctcacagagcctgtccataacc
tgcacagtctctggtttctcattaactagctatggtgtacactgggttcgccagtctccagga
aagggtttaaagtggatgggctggattaatatctactctggaatcccaacatatgctgatgac
ttcaagggacggtttgccttctctttggaaacctctgccagcactgcctatttgcagatcgac
aacctcaaaaatgaggacacggctacatatttctgtgcaagatttgatggtcccgactactgg
ggccaaggcatcactctcactgtctccgca
mAb 7C9: Heavy chain variable region, protein
FR1-CDR1-FR2-CDR2-FR3-CDR3-FR4
(SEQ ID NO: 68)
QVQLKQSGPGLVQPSQSLSITCTVSGFSLTSYGVHWVRQSPGKGLKWMGWINIYSGIPTYADD
FKGRFAFSLETSASTAYLQIDNLKNEDTATYFCARFDGPDYWGQGITLTVSA
Gln Val Gln Leu Lys Gln Ser Gly Pro Gly Leu Val Gln Pro Ser Gln
Ser Leu Ser Ile Thr Cys Thr Val Ser Gly Phe Ser Leu Thr Ser Tyr
Gly Val His Trp Val Arg Gln Ser Pro Gly Lys Gly Leu Lys Trp Met
Gly Trp Ile Asn Ile Tyr Ser Gly Ile Pro Thr Tyr Ala Asp Asp Phe
Lys Gly Arg Phe Ala Phe Ser Leu Glu Thr Ser Ala Ser Thr Ala Tyr
Leu Gln Ile Asp Asn Leu Lys Asn Glu Asp Thr Ala Thr Tyr Phe Cys
Ala Arg Phe Asp Gly Pro Asp Tyr Trp Gly Gln Gly Ile Thr Leu Thr
Val Ser Ala
mAb 7C9: Light chain variable region, DNA
FR1-CDR1-FR2-CDR2-FR3-CDR3-FR4
(SEQ ID NO: 69)
gacattgtgatgacacagtctccatcctccctggctatgtcagtaggacagaaggtcactatg
agctgcaagtccagtcagagccttttaaatagtgtcaatcaaaagaactatttggcctggtac
cagcagaaaccaggacagtctcctaaagttctggtatactttgcatccactagggtatctggg
gtccctgatcgcttcataggcagtggatctgggacagatttcactcttaccatcaccagtgtg
caggctgaagacctgacaacttacttctgtcagcaatattttagcactcctctcacgttcggt
gctgggaccaagctggaactgaaa
mAb 7C9: Light chain variable region, protein
FR1-CDR1-FR2-CDR2-FR3-CDR3-FR4
(SEQ ID NO: 70)
DIVMTQSPSSLAMSVGQKVTMSCKSSQSLLNSVNQKNYLAWYQQKPGQSPKVLVYFASTRVSG
VPDRFIGSGSGTDFTLTITSVQAEDLTTYFCQQYFSTPLTFGAGTKLELK
Asp Ile Val Met Thr Gln Ser Pro Ser Ser Leu Ala Met Ser Val Gly
Gln Lys Val Thr Met Ser Cys Lys Ser Ser Gln Ser Leu Leu Asn Ser
Val Asn Gln Lys Asn Tyr Leu Ala Trp Tyr Gln Gln Lys Pro Gly Gln
Ser Pro Lys Val Leu Val Tyr Phe Ala Ser Thr Arg Val Ser Gly Val
Pro Asp Arg Phe Ile Gly Ser Gly Ser Gly Thr Asp Phe Thr Leu Thr
Ile Thr Ser Val Gln Ala Glu Asp Leu Thr Thr Tyr Phe Cys Gln Gln
Tyr Phe Ser Thr Pro Leu Thr Phe Gly Ala Gly Thr Lys Leu Glu Leu
Lys
mAb 1E4: Heavy chain variable region, DNA
Leader sequence-FR1-CDR1-FR2-CDR2-FR3-CDR3-FR4
(SEQ ID NO: 109)
atgggattcagcaggatctttctcttcctcctgtcagtaactacaggtgtccactcccaggct
tatctacagcagtctggggctgagctggtgaggcctggggcctcagtgaagatgtcctgcaag
gcttttggctacacatttaccagttacaatatgcactgggtgaagcagacacctagacagggc
ctggaatggattggaaccatttatccaggagatggtgacgcttcctacaatcagaaattccag
gacaaggccacactgactgttgacaaatcctccagcacagcctacatgcagctcagcagcctg
acatctgaagactctgcggtctatttctgttcaagggagtttgccgatgcttaccccattccc
ccctttgactactggggccaaggcaccactctcacagtctcctca
mAb 1E4: Heavy chain variable region, protein
Leader sequence-FR1-CDR1-FR2-CDR2-FR3-CDR3-FR4
(SEQ ID NO: 110)
MGFSRIFLFLLSVTTGVHSQAYLQQSGAELVRPGASVKMSCKAFGYTFTSYNMHWVKQTPRQG
LEWIGTIYPGDGDASYNQKFQDKATLTVDKSSSTAYMQLSSLTSEDSAVYFCSREFADAYPIP
PFDYWGQGTTLTVSS
mAb 1E4: Light chain variable region, DNA
Leader sequence-FR1-CDR1-FR2-CDR2-FR3-CDR3-FR4
(SEQ ID NO: 111)
atgaagttgcctgttaggctgttggtgctgattttctggattcctgcttccagtagtgatgtt
ttgatgacccaaactccactctccctggttgtcagtcttggagatcaggcctccatctcttgc
agatctagtcagagcattgtatatagtaatggaaacacctatttagaatggtacctgcaaaaa
ccaggccagtctccaaagctcctgatttacaaagtttccaaccgattttctggggtcccagac
aggttcagtggcagtggatcagggacagatttcacactcaagatcagcagagtggaggctgag
gatctgggagtttattactgctttcaaggttcacatgttccgtacacgttcggaggggggacc
aagctggaaataaaa
mAb 1E4: Light chain variable region, protein
Leader sequence-FR1-CDR1-FR2-CDR2-FR3-CDR3-FR4
(SEQ ID NO: 112)
MKLPVRLLVLIFWIPASSSDVLMTQTPLSLVVSLGDQASISCRSSQSIVYSNGNTYLEWYLQK
PGQSPKLLIYKVSNRFSGVPDRFSGSGSGTDFTLKISRVEAEDLGVYYCFQGSHVPYTFGGGT
KLEIK

### Example 5

### Epitope Mapping of Anti-Canine Pd-1 Antibodies

Introduction

The interaction of antibodies with their cognate protein antigens is mediated through the binding of specific amino acids (paratopes) of the antibodies with specific amino acids (epitopes) of target antigens. An epitope is an antigenic determinant that causes a specific reaction by an immunoglobulin. It consists of a group of amino acids on the surface of the antigen.

A protein of interest may contain several epitopes that are recognized by different antibodies. The epitopes recognized by antibodies are classified as linear or conformational epitopes. Linear epitopes are formed by a stretch of continuous sequence of amino acids in a protein, while conformational epitopes are composed of amino acids that are discontinuous (e.g., far apart) in the primary amino acid sequence, but are brought together upon three-dimensional protein folding.

Epitope mapping refers to the process of identifying the amino acid sequences (i.e., epitopes) that are recognized by antibodies on their target antigens. Identification of epitopes recognized by monoclonal antibodies (mAbs) on target antigens has important applications. For example, it can aid in the development of new therapeutics, diagnostics, and vaccines. Epitope mapping can also aid in the selection of optimized therapeutic mAbs and help elucidate their mechanisms of action. Epitope information can also elucidate unique cancer epitopes and define the protective or pathogenic effects of vaccines.

Epitope mapping can be carried out using polyclonal or monoclonal antibodies and several methods are employed for epitope identification depending on the suspected nature of the epitope (i.e., linear versus conformational). Mapping linear epitopes is more straightforward and relatively easy to perform. For this purpose, commercial services for linear epitope mapping often employ peptide scanning. In this case, an overlapping set of short peptide sequences of the target protein are chemically synthesized and tested for their ability to bind antibodies of interest. The strategy is rapid, high-throughput, and relatively inexpensive to perform. On the other hand, mapping of discontinuous epitope is more technically challenging and requires more specialized techniques such as x-ray co-crystallography of a monoclonal antibody together with its target protein, Hydrogen-Deuterium (H/D) exchange, and/or Mass Spectroscopy coupled with enzymatic digestion.

Mapping of PD-1 Epitopes Using a ProImmune® MicroArray:

In order to identify the amino acids that form the epitopes for anti-PD1 mAbs, a total of 28 peptides that are 15 amino acids long and overlapping by 10 amino acids were chemically synthesized. This library of overlapping peptides was designed to cover the full length canine PD-1 protein. The sequences of these peptides are listed in Table 6 below. The determination of peptide-antibody binding was performed by attachment of antibody samples to the ProArray Ultra® peptide microarray, followed by incubation with a fluorescent-labelled secondary antibody. All peptides are synthesized separately, and then bound to the ProArray Ultra® slide surface alongside ProImmune® murine IgG controls. This optimized process ensures that peptides are presented on the array in such a manner as to closely mimic the properties of the corresponding protein region, circumventing the inherent physiochemical variation of the free peptides themselves and making a compatible, combined peptide and protein array platform. The test analytes (peptides) are dispensed onto the ProArray Ultra® slide in discrete spots and appropriate gal-files enable exact alignment of the resulting array features back to the analyte deposited. ProArray Ultra® slides were blocked using a validated blocking buffer to reduce non-specific binding of the mAbs. They were then incubated with the mAb samples, followed by incubation with a specific fluorescent-labelled secondary antibody. After several washing steps, the ProArray Ultra® arrays were dried and scanned using a high-resolution fluorescence microarray scanning system. After scanning the fluorescent labelled ProArray Ultra® slides, the scanner recorded an image which was evaluated using image analysis software—enabling interpretation and quantification of the levels of fluorescent intensities associated with each fluorescent spot on the scanned microarray slide. The results of this experiment indicated some of the canine PD-1 peptides were recognized by some of the mAbs evaluated. The identity of the mAbs and the amino acid sequence recognized by these mAbs are listed in Table 7. This study indicates that mAb 2H9 recognizes an epitope located in the extracellular domain of canine PD-1 comprised of the amino acid sequence represented by SEQ ID NO: 84 and that mAb 1A1 recognizes an epitope comprising the amino acid sequence represented by SEQ ID NO: 84 and the overlapping amino acid sequence represented by the amino acid sequence represented by SEQ ID NO: 83.

Mapping of PD-1 Epitopes Using Mass Spectroscopy:

In order to identify potentially discontinuous epitopes recognized by anti-canine PD-1 a method based on chemical crosslinking and mass spectrometry detection was used (CovalX® Instrument Incorporated). The application of this technology to epitope mapping of canine PD-1 resulted in identification of at least portions of epitopes recognized by the indicated mAbs which are listed in Table 8. As can be seen from Table 8, mAb 3B6 recognizes at least a portion of an epitope located in the extracellular domain of canine PD-1 within the amino acid sequence represented by SEQ ID NO: 99 and that mAb 2G9 recognizes at least a portion of an epitope within the amino acid sequence represented by SEQ ID NO: 100. On the other hand, mAb 1E4 and mAb 1B5 recognize at least a portion of an epitope within the amino acid sequence represented by SEQ ID NO: 101 and acid sequence represented by SEQ ID NO: 102, respectively.

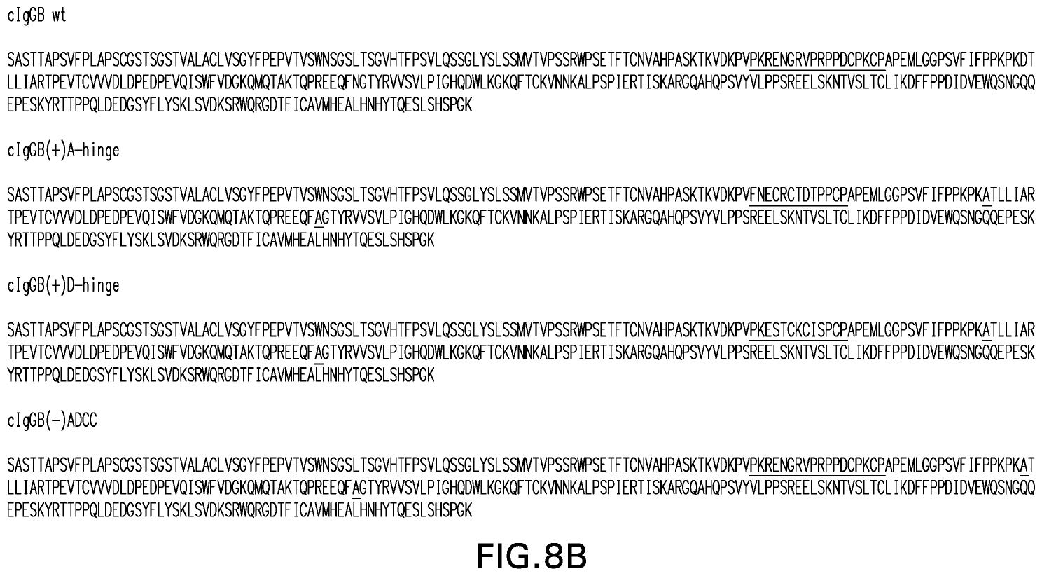

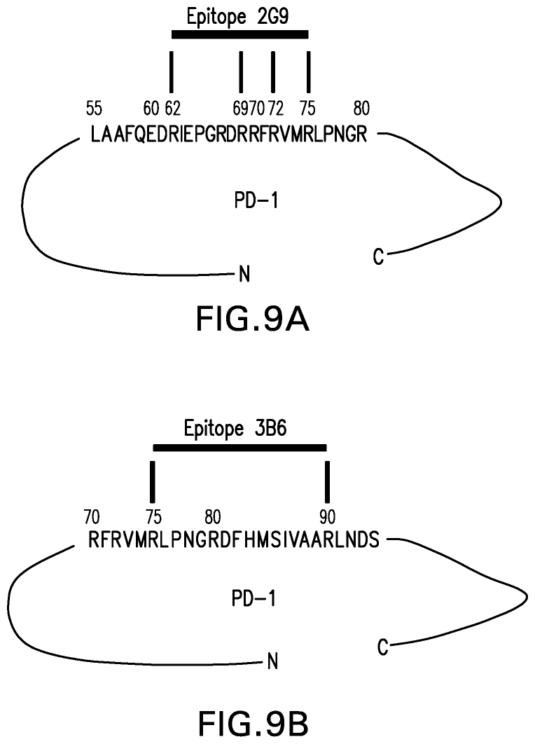

As depicted in FIG. 9A a determination performed by chemical cross-linking, High-Mass MALDI mass spectrometry and nLC-Orbitrap mass spectrometry shows that the epitope on canine PD-1 recognized by caninized antibody 2G9 comprises R₆₂, R₆₉, R₇₂, and R₇₅ of SEQ ID NO: 2. The analogous determination for the epitope on canine PD-1 recognized by caninized antibody 3B6 comprises R₇₅ and R₉₀ of SEQ ID NO: 2. Accordingly, R₇₅ appears to be a particularly important amino acid residue in one or more epitopes of canine PD-1. Interestingly, after performing these analyses, the amino acid sequence for the CDRs of 1A1 were found to be identical to that of 2G9. The consistency between the region on PD-1 that 2G9 binds with that found for 1A1, which were obtained by these two very different methodologies, indicates that this region contains amino acid residues comprised by a PD-1 epitope that is recognized by the anti-canine PD-1 antibodies (see, Tables 7 and 8 below).

Moreover, the region of the amino acid sequence of PD-1 that is recognized by the blocking antibodies of the present invention tested is within the extracellular domain of canine PD-1. The region recognized is comprised by the following peptide (see, Tables 7 and 8 below).

(SEQ ID NO: 103)
NQTDKLAAFQEDRIEPGRDRRFRVM*RLPNGRDFHMSIVAARLNDS

Within this peptide, is a shorter peptide that is in bold. This shorter peptide was recognized with the ProImmune® MicroArray (see, Table 7).

(SEQ ID NO: 104)

DRIEPGRDRRFRVM*RLPNGR

Notably, R₆₂, R₆₉, R₇₂, and R₇₅ of SEQ ID NO: 2 are all comprised by both the longer peptide (SEQ ID NO: 103) and the shorter peptide (SEQ ID NO: 104), whereas R₉₀ of SEQ ID NO: 2 is in the longer peptide. These five arginine residues appear to be important amino acid residues in one or more epitopes of canine PD-1. As indicated in the Tables 6-8, the starred methionine residue (*) has also been reported as being a threonine residue.

TABLE 6
PEPTIDES USED FOR EPITOPE MAPPING
BY PROIMMUNE ® MICROARRAY

| SEQ | |
|---|---|
| ID | |
| NO: | ANTIGEN PEPTIDE |
| 71 | LDSPDRPWSPLTFSP |
| 72 | RPWSPLTFSPAQLTV |
| 73 | LTFSPAQLTVQEGEN |
| 74 | AQLTVQEGENATFTC |
| 75 | QEGENATFTCSLADI |
| 76 | ATFTCSLADIPDSFV |
| 77 | SLADIPDSFVLNWYR |
| 78 | PDSFVLNWYRLSPRN |
| 79 | LNWYRLSPRNQTDKL |
| 80 | LSPRNQTDKLAAFQE |
| 81 | QTDKLAAFQEDRIEP |
| 82 | AAFQEDRIEPGRDRR |
| 83 | DRIEPGRDRRFRVM*R |
| 84 | GRDRRFRVM*RLPNGR |
| 85 | FRVM*RLPNGRDFHMS |
| 86 | LPNGRDFHMSIVAAR |
| 87 | DFHMSIVAARLNDSG |
| 88 | IVAARLNDSGIYLCG |
| 89 | LNDSGIYLCGAIYLP |
| 90 | IYLCGAIYLPPNTQI |
| 91 | AIYLPPNTQINESPR |
| 92 | PNTQINESPRAELSV |
| 93 | NESPRAELSVTERTL |
| 94 | AELSVTERTLEPPTQ |
| 95 | TERTLEPPTQSPSPP |
| 96 | EPPTQSPSPPPRLSG |
| 97 | SPSPPPRLSGQLQGL |
| 98 | PSPPPRLSGQLQGLV |

*This methionine residue has also been reported as being a threonine residue.

TABLE 7
PD-1 EPITOPES RECOGNIZED BY ANTI-CANINE PD-1
MAABS USING PROIMMUNE ® MICROARRAY

| | | SEQ |
|---|---|---|
| | | ID |
| ANTIBODY | ANTIGEN PEPTIDE | NO: |
| 2H9 | GRDRRFRVM*RLPNGR | 84 |
| 1A1^{#} | DRIEPGRDRRFRVM*R | 83 |
| 1A1 | GRDRRFRVM*RLPNGR | 84 |

*This methionine residue has also been reported as being a threonine residue.
^{#}The CDRs of 1A1 are identical to those of 2G9.

TABLE 8
PD-1 EPITOPES RECOGNIZED BY ANTI-CANINE PD-1
MAABS USING MASS SPECTROMETRY

| | | SEQ |
|---|---|---|
| | | ID |
| ANTIBODY | PEPTIDE ANTIGEN | NO: |
| 3B6 | RFRVM*RLPNGRDFHMSIVAARLNDS | 99 |
| 2G9 | LAAFQEDRIEPGRDRRFRVM*RLPNGR | 100 |
| 1E4 | EDRIEPGRDRRFRVM*RLPNGRDFHMSIVAAR | 101 |
| 1B5 | NQTDKLAAFQEDRIEPGRDRRFRVM*RLPNGR | 102 |

*This methionine residue has also been reported as being a threonine residue.

TABLE 9
SEQUENCE LISTING FOR CANINE PD-1 AND PD-L1

| SEQ | | | | SEQ | | | |
|---|---|---|---|---|---|---|---|
| ID | N.A. | A.A. | Description | ID | N.A. | A.A. | Description |
| 1 | ✓ | | Canine PD-1 | 7 | ✓ | | Canine PD-L1 |
| | | | Full Length | | | | Full Length |
| 2 | | ✓ | Canine PD-1 | 8 | | ✓ | Canine PD-L1 |
| | | | Full Length | | | | Full Length |
| 3 | ✓ | | Canine PD-1 | 9 | ✓ | | Canine PD-L1 |
| | | | ECD | | | | ECD |
| 4 | | ✓ | Canine PD-1 | 10 | | ✓ | Canine PD-L1 |
| | | | ECD | | | | ECD |
| 5 | ✓ | | Canine PD-1 - | 11 | ✓ | | Canine PD-L1 - |
| | | | Human IgG1 | | | | Human IgG1 |
| 6 | | ✓ | Canine PD-1 - | 12 | | ✓ | Canine PD-L1 - |
| | | | Human IgG1 | | | | Human IgG1 |
| 105 | ✓ | | Full Length Canine | 107 | ✓ | | Full Length Canine |
| | | | PD-1 + signal | | | | PD-L1 + signal |
| | | | sequence | | | | sequence |
| 106 | | ✓ | Full Length Canine | 108 | | ✓ | Full Length Canine |
| | | | PD-1 + signal | | | | PD-L1 + signal |
| | | | sequence | | | | sequence |
| 113 | | ✓ | Canine PD-1 - | | | | |
| | | | Hum. IgG1 + sig. seq | | | | |

TABLE 10
SEQUENCE LISTING TABLE FOR
CANINE IgGB MODIFICATIONS

| ID | N.A. | A.A. | Description |
|---|---|---|---|
| 39 | | ✓ | cIgGB wt |
| 40 | | ✓ | cIgGB(+)A-hinge |
| 41 | | ✓ | cIgGB(+)D-hinge |
| 42 | | ✓ | cIgGB(−)ADCC |

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1-8. (canceled)

9. The isolated mammalian antibody or antigen binding fragment thereof of claim 35 wherein the mammalian antibody is a murine antibody.

10. The isolated mammalian antibody or antigen binding fragment thereof of claim 9 that is a caninized antibody.

11-12. (canceled)

13. The isolated mammalian antibody or antigen binding fragment thereof of claim 9, wherein when bound to canine PD-1, said antibody binds to at least one amino acid residue within SEQ ID NO: 103; wherein the antibody and antigen binding fragment thereof binds canine PD-1 and blocks the binding of canine PD-1 to canine Programmed Death Ligand 1 (PD-L1).

14. The isolated mammalian antibody or antigen binding fragment thereof of claim 13 wherein when bound to canine PD-1 said antibody binds to at least one amino acid residue within one or more amino acid sequences selected from the group consisting of SEQ ID NO: 83, SEQ ID NO: 84, SEQ ID NO: 99, SEQ ID NO: 100, of SEQ ID NO: 101, SEQ ID NO: 102, and SEQ ID NO: 104.

15. (canceled)

16. The isolated mammalian antibody or antigen binding fragment thereof of claim 14 wherein when bound to canine PD-1 said antibody binds to one or more amino acid residues selected from the group consisting of R₆₂, R₆₉, R₇₂, R₇₅, and R₉₀ of SEQ ID NO: 2.

17. A monoclonal antibody or antigen binding fragment thereof that cross-competes for binding with canine PD-1 with one or more mammalian antibody of claim 9; wherein the antibody and antigen binding fragment thereof bind canine PD-1 and block the binding of canine PD-1 to PD-L1.

18. An isolated nucleic acid that encodes the light chain of the antibody of claim 35.

19. An isolated nucleic acid that encodes the heavy chain of the antibody of claim 35.

20. An isolated nucleic acid of that encodes one or more amino acid sequences selected from the group consisting of SEQ ID NOs: 13, 14, 15, 16, 17, 18, 19, 20, 21, 22, 23, 24, 25, 26, 27, 28, 29, 30, 31, 32, 33, 34, 35, 36, 37, 38, or 114.

21. An expression vector comprising the isolated nucleic acid of claim 19.

22. (canceled)

23. An isolated antigenic peptide consisting of 60 or fewer amino acid residues that comprises the amino acid sequence of SEQ ID NO: 103.

24. (canceled)

25. The antigenic peptide of claim 23, wherein said amino acid sequence is selected from the group consisting of SEQ ID NO: 83, SEQ ID NO: 84, SEQ ID NO: 99, SEQ ID NO: 100, SEQ ID NO: 101, SEQ ID NO: 102, and SEQ ID NO: 104.

26-31. (canceled)

32. A pharmaceutical composition comprising the antibody of claim 35, and a pharmaceutically acceptable carrier or diluent.

33. A method of increasing the activity of an immune cell, comprising administering to a subject in need thereof a therapeutically effective amount of the pharmaceutical composition of claim 32.

34. (canceled)

35. An isolated mammalian antibody or an antigen binding fragment thereof that binds canine Programmed Death Receptor 1 (canine PD-1) with specificity, said antibody comprising three light chain complementary determining regions (CDRs): CDR light 1 (CDRL1), CDR light 2 (CDRL2), and CDR light 3 (CDRL3); and three heavy chain CDRs: CDR heavy 1 (CDRH1), CDR heavy 2 (CDRH2) and CDR heavy 3 (CDRH3):
(a) wherein CDRL1 comprises an amino acid sequence selected from the group consisting of SEQ ID NO: 15, a conservatively modified variant of SEQ ID NO: 15, a variant of SEQ ID NO: 15 that comprises the canonical structure class of 2A; SEQ ID NO: 13, a conservatively modified variant of SEQ ID NO: 13, a variant of SEQ ID NO: 13 that comprises the canonical structure class of 3; SEQ ID NO: 14, a conservatively modified variant of SEQ ID NO: 14, and a variant of SEQ ID NO: 14 that comprises the canonical structure class of 4;
(b) wherein CDRL2 comprises an amino acid sequence selected from the group consisting of SEQ ID NO: 18, SEQ ID NO: 16, SEQ ID NO: 17, SEQ ID NO 19, SEQ ID NO: 20, SEQ ID NO: 21, a conservatively modified variant of SEQ ID NO 18, SEQ ID NO: 16, SEQ ID NO: 17, SEQ ID NO: 19, SEQ ID NO: 20, SEQ ID NO: 21, and a variant of SEQ ID NO: 18, SEQ ID NO: 16, SEQ ID NO: 17, SEQ ID NO: 19, SEQ ID NO: 20, SEQ ID NO: 21 that comprises the canonical structure class of 1;
(c) wherein CDRL3 comprises an amino acid sequence selected from the group consisting of SEQ ID NO: 24, SEQ ID NO 22, SEQ ID NO: 23, SEQ ID NO 25, SEQ ID NO: 26, a conservatively modified variant of SEQ ID NO 24, SEQ ID NO: 22, SEQ ID NO: 23, SEQ ID NO: 25, SEQ ID NO: 26, and a variant of SEQ ID NO: 24, SEQ ID NO: 22, SEQ ID NO: 23, SEQ ID NO: 25, SEQ ID NO: 26 that comprises the canonical structure class of 1;
(d) wherein CDRH1 comprises an amino acid sequence selected from the group consisting of SEQ ID NO: 29, SEQ ID NO: 27, SEQ ID NO: 28, SEQ ID NO: 25, SEQ ID NO: 30, a conservatively modified variant of SEQ ID NO: 29, SEQ ID NO: 27, SEQ ID NO: 28, SEQ ID NO: 30, and a variant of SEQ ID NO: 29, SEQ ID NO: 27, SEQ ID NO: 28, SEQ ID NO: 30 that comprises the canonical structure class of 1;
(e) wherein CDRH2 comprises an amino acid sequence selected from the group consisting of SEQ ID NO: 33, SEQ ID NO: 31, SEQ ID NO: 32, SEQ ID NO: 34, SEQ ID NO: 35, a conservatively modified variant of SEQ ID NO: 33, SEQ ID NO: 31, SEQ ID NO: 32, SEQ ID NO: 34, SEQ ID NO: 35, a variant of SEQ ID NO: 33 or SEQ ID NO: 32 that comprises the canonical structure class of 2A; and a variant of SEQ ID NO: 31, SEQ ID NO: 34, SEQ ID NO: 35 that comprises the canonical structure class of 1;
(f) wherein CDRH3 comprises an amino acid sequence selected from the group consisting of SEQ ID NO: 38, a conservatively modified variant of SEQ ID NO: 38, a variant of SEQ ID
NO: 38 that comprises the canonical structure class of 11, SEQ ID NO: 36, a conservatively modified variant of SEQ ID NO 36, a variant of SEQ ID NO 36 that comprises the canonical structure class of 6; SEQ ID NO: 37, a conservatively modified variant of SEQ ID NO: 37, a variant of SEQ ID NO: 37 that comprises the canonical structure class of 13; SEQ ID NO: 114, a conservatively modified variant of SEQ ID NO: 114, and a variant of SEQ ID NO: 114 that comprises the canonical structure class of 11; and
wherein the antibody and the antigen binding fragment thereof bind canine PD-1 and block the binding of canine PD-1 to canine Programmed Death Ligand 1 (PD-LI).

36. The isolated mammalian antibody or the antigen binding fragment thereof of claim 35,
a) wherein CDRL1 comprises an amino acid sequence selected from the group consisting of SEQ ID NO: 15, a conservatively modified variant of SEQ ID NO: 15, and a variant of SEQ ID NO: 15 that comprises the canonical structure class of 2A;
(b) wherein CDRL2 comprises an amino acid sequence selected from the group consisting of SEQ ID NO: 18, a conservatively modified variant of SEQ ID NO 18, and a variant of SEQ ID NO: 18 that comprises the canonical structure class of 1;
(c) wherein CDRL3 comprises an amino acid sequence selected from the group consisting of SEQ ID NO: 24, a conservatively modified variant of SEQ ID NO: 24, and a variant of SEQ ID NO: 24 that comprises the canonical structure class of 1;
(d) wherein CDRH1 comprises an amino acid sequence selected from the group consisting of SEQ ID NO: 29, a conservatively modified variant of SEQ ID NO: 29, and a variant of SEQ ID NO: 29 that comprises the canonical structure class of 1;
(e) wherein CDRH2 comprises an amino acid sequence selected from the group consisting of SEQ ID NO: 33, a conservatively modified variant of SEQ ID NO 33, and a variant of SEQ ID NO: 33 that comprises the canonical structure class of 2A; and
(f) wherein CDRH3 comprises an amino acid sequence selected from the group consisting of SEQ ID NO: 38, a conservatively modified variant of SEQ ID NO: 38, and a variant of SEQ ID NO: 38 that comprises the canonical structure class of 11.

37. A pharmaceutical composition comprising the antibody of claim 36 and a pharmaceutically acceptable carrier or diluent.

38. The isolated mammalian antibody or the antigen binding fragment thereof of claim 35,
(a) wherein CDRL1 comprises an amino acid sequence selected from the group consisting of SEQ ID NO: 13, a conservatively modified variant of SEQ ID NO: 13, and a variant of SEQ ID NO: 13 that comprises the canonical structure class of 3;
(b) wherein CDRL2 comprises an amino acid sequence selected from the group consisting of SEQ ID NO: 19, a conservatively modified variant of SEQ ID NO: 19, and a variant of SEQ ID NO: 19 that comprises the canonical structure class of 1;
(c) wherein CDRL3 comprises an amino acid sequence selected from the group consisting of SEQ ID NO: 25, a conservatively modified variant of SEQ ID NO: 25, and a variant of SEQ ID NO: 25 that comprises the canonical structure class of 1;
(d) wherein CDRH1 comprises an amino acid sequence selected from the group consisting of SEQ ID NO: 27, a conservatively modified variant of SEQ ID NO: 27, and a variant of SEQ ID NO: 27 that comprises the canonical structure class of 1;
(e) wherein CDRH2 comprises an amino acid sequence selected from the group consisting of SEQ ID NO: 31, a conservatively modified variant of SEQ ID NO: 31, and a variant of SEQ ID NO: 31 that comprises the canonical structure class of 1;
(f) wherein CDRH3 comprises an amino acid sequence selected from the group consisting of SEQ ID NO: 36, a conservatively modified variant of SEQ ID NO: 36, and a variant of SEQ ID NO: 36 that comprises the canonical structure class of 6.

39. A pharmaceutical composition comprising the antibody of claim 38 and a pharmaceutically acceptable carrier or diluent.

40. An expression vector comprising the isolated nucleic acid of claim 18.
